(19) United States
(12) Patent Application Publication
(10) Pub. No.: US 2024/0024317 A1
(43) Pub. Date: Jan. 25, 2024

(54) CANCER TREATMENT USING BOSENTAN IN COMBINATION WITH A CHECKPOINT INHIBITOR

(71) Applicants: Materia Therapeutics Inc., Las Vegas, NV (US); University of Cyprpus, Nicosia (CY)

(72) Inventors: John D. MARTIN, Nicosia (CY); Triantafyllos STYLIANOPOULOS, Nicosia (CY); Fotios MPEKRIS, Nicosia (CY); Myrofora PANAGI, Nicosia (CY); Chysovalantis VOUTOURI, Nicosia (CY); Andreas STYLIANOU, Nicosia (CY)

(21) Appl. No.: 18/256,776

(22) PCT Filed: Dec. 9, 2021
(86) PCT No.: PCT/US2021/062663

§ 371 Date: Jun. 9, 2023

Related U.S. Application Data
(60) Provisional application No. 63/124,448, filed on Dec. 11, 2020.

(51) Int. Cl. A61K 31/506; C07K 16/28; A61P 35/00

(52) U.S. Cl. CPC A61K 31/506; C07K 16/2827; C07K 16/2818; A61P 35/00; A61K 2039/507

(57) ABSTRACT

The present invention is directed to a combination treatment using bosentan and a checkpoint inhibitor that is effective in treating cancer or inhibiting the proliferation of tumor cells in a subject and/or that can initiate, enhance or prolong the immune response to tumor cells.

### CROSS-REFERENCE TO RELATED APPLICATIONS

This application claims priority benefit to U.S. Provisional Application No. 63/124,448, filed on Dec. 11, 2020, entitled “CANCER TREATMENT USING BOSENTAN IN COMBINATION WITH A CHECKPOINT INHIBITOR”, the contents of which are incorporated herein by reference for all purposes.

### FIELD OF THE INVENTION

The present invention discloses a combination treatment using bosentan and a checkpoint inhibitor that is effective in treating cancer or inhibiting the proliferation of tumor cells in a subject and/or that can initiate, enhance or prolong the immune response to tumor cells.

### BACKGROUND

Efficacy of cancer immunotherapy depends on whether T cells can traffic to tumors and migrate to a location adjacent to malignant cells to recognize and kill them. One barrier to T cell homing is the tumor blood vessel wall, which inhibits T cell attachment and transmigration through the endothelin B receptor, but antagonizing this receptor has not yet led to a clinically approved drug. One reason could be hypoperfusion in tumors, which could limit the surface area of perfused blood vessels for anti-tumor T cells to attach. If collapsed tumor blood vessels could be decompressed and reperfused by alleviating mechanical compression (i.e. solid stress), endothelin B receptor antagonism could increase the efficacy of cancer immunotherapy.

Bosentan (Tracleer®; Stayveer®) is a dual endothelin receptor antagonist used in the treatment of pulmonary artery hypertension (PAH). Bosentan is a competitive antagonist of endothelin-1 at the endothelin-A (ET-A) and endothelin-B (ET-B) receptors. Under normal conditions, endothelin-1 binding of ET-A receptors causes constriction of the pulmonary blood vessels. Conversely, binding of endothelin-1 to ET-B receptors has been associated with both vasodilation and vasoconstriction of vascular smooth muscle, depending on the ET-B subtype (ET-B1 or ET-B2) and tissue. Bosentan blocks both ET-A and ET-B receptors, but is thought to exert a greater effect on ET-A receptors, causing a total decrease in pulmonary vascular resistance.

Immune checkpoints, which act as the off-switch on the T cells of the immune system, have been investigated to reinstate the immune response with targeted agents, thus indirectly treating cancer by activating the body's immune system.

International applications WO2002086083, WO2004004771, WO2004056875, WO2006121168, WO2008156712, WO2010077634, WO2011066389, WO2014055897 and WO2014100079 report PD-1, PD-L1 inhibitory antibodies and/or methods of identifying such antibodies. Further, U.S. patents such as U.S. Pat. Nos. 8,735,553 and 8,168,757 report PD-1 or PD-L1 inhibitory antibodies and/or fusion proteins. The disclosures of WO2002086083, WO2004004771, WO2004056875, WO2006121168, WO2008156712, WO2010077634, WO2011066389, WO2014055897 and WO2014100079, as well as U.S. Pat. Nos. 8,735,553 and 8,168,757, are incorporated herein by reference in their entirety.

Moreover, International applications WO2011161699, WO2012168944, WO2013144704, WO2013132317 and WO 2016044900 report peptides or peptidomimetic compounds which are capable of suppressing and/or inhibiting the programmed cell death 1 (PD-1) signaling pathway. The disclosures of WO2011161699, WO2012168944, WO2013144704, WO2013132317 and WO 2016044900 are incorporated herein by reference in their entirety.

Furthermore, International applications WO 2016142852, WO 2016142894, WO 2016142886, WO 2016142835 and WO 2016142833 report small molecule compounds which are capable of suppressing and/or inhibiting the programmed cell death 1 (PD-1) signaling pathway and/or treating disorders by inhibiting an immunosuppressive signal induced by PD-1, PD-L1 or PD-L2. The disclosures of WO 2016142852, WO 2016142894, WO 2016142886, WO 2016142835 and WO 2016142833 are incorporated herein by reference in their entirety.

Recently, ipilimumab (Yervoy®), a monoclonal antibody that targets cytotoxic T-lymphocyte-associated antigen 4 (CTLA-4) and nivolumab (Opdivo®), a monoclonal antibody that targets the programmed cell death protein 1 pathway (PD-1) on the surface of T-cells, have been approved by the U.S. Food and Drug Administration for the treatment of advanced melanoma, advanced renal cell carcinoma, and non-small cell lung cancer. Current checkpoint inhibitor therapies, however, are effective at treating cancer in a relatively small population of cancer subject population, which is in part due to pre-existing immune activation and presence of the inhibitory receptors. Although immune checkpoint blockade (ICB) with checkpoint inhibitors has revolutionized treatment for many types of solid tumors, it is now estimated to benefit less than 20% of cancer patients. Increasing the fraction of patients responding and the length of their response is an urgent unmet clinical need. Accordingly, there is a need to develop methods and combination therapies to initiate or enhance the effectiveness of the checkpoint inhibitors in both the nonresponding subject population and the responding subject population.

Anti-tumor T cells must circulate into tumors through blood vessels, bind the endothelium, and pass across the vessel wall and migrate through cancer-associated fibroblasts (CAFs) and extracellular matrix (ECM) before encountering cancer cells. However, because up to 80% of intratumoral blood vessels lack perfusion, the area of vessel wall for T cells to migrate across is limited.

Compressed blood vessels impair blood flow and oxygen delivery to tumors, resulting in increased hypoxia in the tumors and resistance to immunotherapy through numerous mechanisms. Strategies that decompress vessels potentiate the efficacy of ICB in ICB-resistant mouse models of metastatic breast cancer. If there was a way to decompress tumor vessels while also facilitating vessel adhesion and transmigration of T cells into the tumor parenchyma, the fraction of cancer patients that respond to ICB could increase.

All references cited herein, including patent applications, patent publications, and scientific literature, are herein incorporated by reference in their entirety, as if each individual reference were specifically and individually indicated to be incorporated by reference.

### SUMMARY

Provided herein is a method for treating a solid tumor in a subject in need thereof comprising administering to the subject an effective amount of bosentan, or a pharmaceutically acceptable salt thereof, in combination with a checkpoint inhibitor. Also provided herein is a method for initiating, enhancing or prolonging the effects of a checkpoint inhibitor, or enabling a subject to respond to a checkpoint inhibitor in a subject in need thereof comprising administering to the subject an effective amount of bosentan, or a pharmaceutically acceptable salt thereof, in combination with a checkpoint inhibitor, wherein the subject has a solid tumor. Also provided herein is a method for potentiating the effects of a checkpoint inhibitor in a subject in need thereof comprising administering to the subject an effective amount of bosentan, or a pharmaceutically acceptable salt thereof, in combination with a checkpoint inhibitor, wherein the subject has a solid tumor. Also provided herein is method of increasing blood flow of a solid tumor in a subject comprising administering to the subject an effective amount of bosentan, or a pharmaceutically acceptable salt thereof, in combination with a checkpoint inhibitor, wherein increasing blood flow of the solid tumor enhances the effect of the checkpoint inhibitor. In some embodiments, blood flow is measured using ultrasound-based blood flow measurements or using histological techniques to measure hypoxia. In some embodiments, blood flow is measured using ultrasound-based blood flow measurements. In some embodiments, blood flow is measured using histological techniques to measure hypoxia. In some embodiments, blood flow is measured using histological techniques to measure hypoxia in a biopsy from the solid tumor. Also provided herein is a method of improving the delivery or efficacy of a checkpoint inhibitor in a subject comprising administering an effective amount of bosentan, or a pharmaceutically acceptable salt thereof, in combination with the checkpoint inhibitor, wherein the subject has a solid tumor, thereby improving the delivery or efficacy of the therapy in the subject. In some embodiments, administering bosentan, or pharmaceutically acceptable salt thereof, increases the number of anti-tumor T cells that colocalize with the solid tumor. In some embodiments, administering bosentan, or pharmaceutically acceptable salt thereof, reduces the tissue stiffness of the solid tumor. In some embodiments, the tissue stiffness of the solid tumor is measured using ultrasound elastography. In some embodiments, administering bosentan, or pharmaceutically acceptable salt thereof, decreases the levels of an extracellular matrix protein in the solid tumor. In some embodiments, the extracellular matrix protein is collagen I or hyaluronan binding protein (HABP). In some embodiments, administering bosentan, or pharmaceutically acceptable salt thereof, reduces hypoxia in the solid tumor. In some embodiments, the checkpoint inhibitor inhibits a checkpoint protein selected from the group consisting of CTLA-4, PD-1, PD-L1, PD-L2, B7-H3, B7-H4, BMA, HVEM, TIM3, GALS, LAG3, VISTA, KIR, 2B4, CD160, CGEN-15049, CHK1, CHK2, A2aR, and B-7 family ligands or a combination thereof. In some embodiments, the checkpoint inhibitor is a CTLA-4, PD-L1, PD-L2, or PD-1 inhibitor. In some embodiments, the checkpoint inhibitor is an anti-PD-1 antibody, anti-PD-L1 antibody, or anti-CTLA4 antibody. In some embodiments, the checkpoint inhibitor is selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor is a combination of an anti-PD-1 antibody and an anti-CTLA-4 antibody. In some embodiments, the bosentan, or pharmaceutically acceptable salt thereof, is administered to the subject once per day. In some embodiments, the bosentan, or pharmaceutically acceptable salt thereof, is administered to the subject twice per day. In some embodiments, the bosentan, or pharmaceutically acceptable salt thereof, is administered to the subject at a dose from about mg/kg to about 5 mg/kg. In some embodiments, the bosentan, or pharmaceutically acceptable salt thereof, is administered to the subject at a dose from about 100 mg to about 1200 mg. In some embodiments, the bosentan, or pharmaceutically acceptable salt thereof, is administered to the subject at a dose from about 125 mg to about 500 mg. In some embodiments, the bosentan, or pharmaceutically acceptable salt thereof, is administered to the subject at a dose of about 125 mg. In some embodiments, the bosentan, or pharmaceutically acceptable salt thereof, is administered to the subject at a dose of about 500 mg. In some embodiments, the bosentan, or pharmaceutically acceptable salt thereof, is administered to the subject prior to the subject being administered the checkpoint inhibitor. In some embodiments, the bosentan, or pharmaceutically acceptable salt thereof, is administered to the subject beginning at least 1 day prior to the subject being administered the checkpoint inhibitor. In some embodiments, the bosentan, or pharmaceutically acceptable salt thereof, is administered to the subject beginning at least 2 days prior to the subject being administered the checkpoint inhibitor. In some embodiments, the bosentan, or pharmaceutically acceptable salt thereof, is administered to the subject beginning at least 3 days prior to the subject being administered the checkpoint inhibitor. In some embodiments, the bosentan, or pharmaceutically acceptable salt thereof, is administered to the subject beginning at least 5 days prior to the subject being administered the checkpoint inhibitor. In some embodiments, the administration of bosentan, or pharmaceutically acceptable salt thereof, to the subject is maintained for at least a portion of the time the subject is administered the checkpoint inhibitor. In some embodiments, the administration of bosentan, or pharmaceutically acceptable salt thereof, to the subject is maintained for the entire period of time the subject is administered the checkpoint inhibitor. In some embodiments, one or more therapeutic effects in the subject is improved after administration of the bosentan, or pharmaceutically acceptable salt thereof, and the checkpoint inhibitor relative to a baseline. In some embodiments, the one or more therapeutic effects is selected from the group consisting of: size of a tumor derived from the cancer, objective response rate, duration of response, time to response, progression free survival and overall survival. In some embodiments, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the bosentan, or pharmaceutically acceptable salt thereof, and the checkpoint inhibitor. In some embodiments, the objective response rate is at least about 20%, at least about 25%, at least about 30%, at least about 35%, at least about 40%, at least about 45%, at least about 50%, at least about 60%, at least about 70%, or at least about 80%. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the bosentan, or pharmaceutically acceptable salt thereof, and the checkpoint inhibitor. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the bosentan, or pharmaceutically acceptable salt thereof, and the checkpoint inhibitor. In some embodiments, the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the bosentan, or pharmaceutically acceptable salt thereof, and the checkpoint inhibitor. In some embodiments, the solid tumor is selected from the group consisting of breast cancer, breast cancer lung metastases, sarcoma, pancreatic cancer, ovarian cancer, liver metastases, prostate cancer, brain cancer, melanoma, renal cell carcinoma, colorectal cancer, hepatocellular carcinoma, lung cancer, squamous cell carcinoma of the head and neck, urothelial carcinoma, esophageal squamous cell carcinoma, gastric cancer, esophageal cancer, cervical cancer, Merkel cell carcinoma, endometrial carcinoma, mesothelioma, and cutaneous squamous cell carcinoma. In some embodiments, the solid tumor is breast cancer. In some embodiments, the breast cancer is triple negative breast cancer. In some embodiments, the subject is a human.

Also provided herein is a kit comprising an effective amount of bosentan, or a pharmaceutically acceptable salt thereof, an effective amount of a checkpoint inhibitor, and instructions for using the bosentan, or a pharmaceutically acceptable salt thereof and the checkpoint inhibitor according to any of the methods described herein.

Also provided herein is a method of determining an effective amount of an agent that decompresses blood vessels in a subject with a solid tumor comprising (a) measuring the blood flow and/or stiffness of the solid tumor; (b) administering to the subject an effective amount of an agent that decompresses blood vessels; and (c) measuring the blood flow and/or stiffness of the solid tumor after administering the agent that decompresses blood vessels, wherein an increase in blood flow and/or a decrease in stiffness following administration of the agent that decompresses blood vessels to the subject indicates that the amount administered was an effective amount. Also provided herein is a method for treating a solid tumor in a subject in need thereof comprising (a) measuring the blood flow and/or stiffness of the solid tumor; (b) administering to the subject an effective amount of an agent that decompresses blood vessels; (c) measuring the blood flow and/or stiffness of the solid tumor after administering the agent that decompresses blood vessels; and (d) administering a chemotherapeutic agent if the blood flow of the solid tumor is increased and/or the stiffness of the solid tumor is decreased after administering the agent that decompresses blood vessels. Also provided herein is a method for treating a solid tumor in a subject in need thereof comprising (a) measuring the blood flow and/or stiffness of the solid tumor; (b) administering to the subject an effective amount of an agent that decompresses blood vessels; (c) measuring the blood flow and/or stiffness of the solid tumor after administering the agent that decompresses blood vessels; (d) determining that the subject is responsive to a chemotherapeutic agent based on an increase in the blood flow of the solid tumor or a decrease in the stiffness of the solid tumor after administering the agent that decompresses blood vessels; and (e) administering the chemotherapeutic agent to the subject who has been determined to be responsive to the chemotherapeutic agent based on the increase in the blood flow of the solid tumor or the decrease in the stiffness of the solid tumor after administering the agent that decompresses blood vessels. Also provided herein is a method for predicting response to treatment with a chemotherapeutic agent comprising (a) measuring the blood flow and/or stiffness of the solid tumor; (b) administering to the subject an effective amount of an agent that decompresses blood vessels; (c) measuring the blood flow and/or stiffness of the solid tumor after administering the agent that decompresses blood vessels, wherein an increase in the blood flow of the solid tumor or a decrease in the stiffness of the solid tumor after administering the agent that decompresses blood vessels indicates that the subject is likely to respond to treatment with the chemotherapeutic agent. In some embodiments, the effective amount of an agent that decompresses blood vessels is determined by measuring the change in blood flow and/or stiffness of the solid tumor following administration of the agent that decompresses blood vessels to the subject, wherein an increase in blood flow and/or a decrease in stiffness following administration of the agent that decompresses blood vessels to the subject indicates that the amount administered was an effective amount. In some embodiments, the method comprises measuring the blood flow of the solid tumor and the blood flow of the solid tumor is increased after administering the agent that decompresses blood vessels. In some embodiments, the method comprises measuring the stiffness of the solid tumor and the stiffness of the solid tumor is decreased after administering the agent that decompresses blood vessels. In some embodiments, the agent that decompresses blood vessels is administered for at least 1 day, at least 2 days, at least 3 days, at least 4 days, or at least 5 days prior to the administration of the chemotherapeutic agent. In some embodiments, the agent that decompresses blood vessels is administered at a dose that increases the blood flow of the solid tumor and/or decreases the stiffness of the solid tumor. In some embodiments, the agent that decompresses blood vessels is bosentan, or a pharmaceutically acceptable salt thereof. In some embodiments, blood flow and/or stiffness of the solid tumor is measured using ultrasound. In some embodiments, blood flow of the solid tumor is measured using histological techniques to measure hypoxia. In some embodiments, the chemotherapeutic agent is a checkpoint inhibitor. In some embodiments, the checkpoint inhibitor inhibits a checkpoint protein selected from the group consisting of CTLA-4, PD-1, PD-L1, PD-L2, B7-H3, B7-H4, BMA, HVEM, TIM3, GALS, LAG3, VISTA, KIR, 2B4, CD160, CGEN-15049, CHK1, CHK2, A2aR, and B-7 family ligands or a combination thereof. In some embodiments, the checkpoint inhibitor is a CTLA-4, PD-L1, PD-L2, or PD-1 inhibitor. In some embodiments, the checkpoint inhibitor is an anti-PD-1 antibody, anti-PD-L1 antibody, or anti-CTLA4 antibody. In some embodiments, the checkpoint inhibitor is selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor is a combination of an anti-PD-1 antibody and an anti-CTLA-4 antibody. In some embodiments, the solid tumor is selected from the group consisting of breast cancer, breast cancer lung metastases, sarcoma, pancreatic cancer, ovarian cancer, liver metastases, prostate cancer, brain cancer, melanoma, renal cell carcinoma, colorectal cancer, hepatocellular carcinoma, lung cancer, squamous cell carcinoma of the head and neck, urothelial carcinoma, esophageal squamous cell carcinoma, gastric cancer, esophageal cancer, cervical cancer, Merkel cell carcinoma, endometrial carcinoma, mesothelioma, and cutaneous squamous cell carcinoma. In some embodiments, the solid tumor is breast cancer. In some embodiments, the breast cancer is triple negative breast cancer. In some embodiments, the subject is a human.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIGS. 1A-1L is a series of images and graphs showing that bosentan normalizes the tumor mechanical microenvironment. FIG. 1A: Ultrasound elastography heat maps of control treated (top) and 1 mg/kg bosentan treated E0771 tumors on day 10 after treatment, with lower kPa indicating compliant tissue and higher kPa indicating stiff tissue. The dashed black line denotes the tumor border. FIG. 1B: Longitudinal quantification of elasticity in E0771 tumors. Symbol indicates P<0.05. FIG. 1C: Longitudinal quantification of elasticity in 4T1 tumors. Symbol indicates P<0.05. FIG. 1D: Young's modulus quantification of atomic force microscopy measurements in E0771 tumors. Symbol indicates P<0.05. FIG. 1E: Representative atomic force microscopy stiffness fingerprint histogram of a control treated E0771 tumor. The peak on the left side of the graph is the contribution from compliant cancer cells, while the tail in the box is the contribution of stiffer components like collagen. FIG. 1F: Representative atomic force microscopy stiffness fingerprint histogram of a 1 mg/kg bosentan treated E0771 tumor. FIG. 1G: Representative atomic force microscopy stiffness fingerprint histogram of a 10 mg/kg bosentan treated E0771 tumor. FIG. 1H: Quantification of interstitial fluid pressure in E0771 tumors. Symbol indicates P<0.05. FIG. 1I: Representative images of pico sirius red staining, aSMA immunofluorescence staining, and hyaluronan binding protein (HABP) immunofluorescence staining in E0771 tumors. FIG. 1J: Quantification of pico sirius red staining in E0771 tumors. Symbol indicates P<0.05. FIG. 1K: Quantification of aSMA staining in 4T1 tumors. Symbol indicates P<0.05. FIG. 1L Quantification of HABP staining in E0771 tumors.

FIGS. 2A-2F is a series of images and graphs showing that bosentan reduces hypoxia and increases T cell association with blood vessels. FIG. 2A: Representative images of pimonidazole (hypoxia) staining (top box) and colocalization of CD3⁺ T cells and CD31⁺ endothelial cells (bottom box) in E0771 tumors. FIG. 2B: Quantification of hypoxic area fraction in 4T1 tumors. Symbol indicates P<0.05. FIG. 2C: Quantification of proximity between CD3⁺ T cells and CD31⁺ endothelial cells in 4T1 tumors. Symbol indicates P<0.05. FIG. 2D: Quantification of fraction of CD3⁺ area in 4T1 tumors. Symbol indicates P<0.05. FIG. 2E: Quantification of fraction of CD31⁺ area in 4T1 tumors. Symbol indicates P<0.05. FIG. 2F: Quantification of mRNA expression in 4T1 tumors. Symbol indicates P<0.05.

FIGS. 3A-3E is a series of graphs showing that bosentan potentiates immune checkpoint blockade (ICB) efficacy in triple negative breast cancer (TNBC). FIG. 3A: Tumor growth curves of E0771 tumors. Mice were treated with control (black), TME-normalizing 1 mg/kg bosentan monotherapy (purple), ICB cocktail of anti-PD-1 and anti-CTLA-4 (green) or the combination (orange), which significantly slowed tumor growth. n=10. Symbol indicates P<0.05. FIG. 3B: Tumor growth curves in 4T1 tumors. Only the combination significantly slowed tumor growth. n=8-10. Symbol indicates P<0.05. FIG. 3C: Kaplan-Meier survival curves of mice bearing spontaneous E0771 metastases arising from surgically removed primary tumors. All mice died before 80 days after inoculation, except for 80% of combination treated mice. n=10. Symbol indicates P<0.05. FIG. 3D: Kaplan-Meier survival curves of mice bearing spontaneous 4T1 metastases arising from surgically removed primary tumors. Only combination treated mice had longer median overall survival. n=8-10. Symbol indicates P<0.05. FIG. 3E: Tumor growth curves of surviving mice rechallenged with E0771 cancer cells versus control mice naïve to E0771 cancer cells.

FIGS. 4A-4B is a series of graphs showing that stiffness and tumor response to ICB correlate. FIG. 4A: Correlation of elastic Young's modulus before the ICB treatment in mice bearing E0771 tumors (n=5-6 mice per group), that were treated either with the ICB cocktail alone or with bosentan and ICB combination therapy to the tumor volume after the completion of treatment (R²=0.9657, p<0.0001). FIG. 4B: Correlation of elastic Young's modulus before the ICB treatment in mice bearing 4T1 tumors (n=5-6 mice per group), that were treated either with the ICB cocktail alone or with bosentan and ICB combination therapy to the tumor volume after the completion of treatment (R²=0.9387, p<0.0001).

FIGS. 5A-5C shows a mouse tumor model for treatment with bosentan plus anti-PD-1/anti-CTLA-4 therapy or anti-PD-1/anti-CTLA-4 therapy alone. FIG. 5A: schematic of the study. FIG. 5B: effects of bosentan in combination with antibody therapy or antibody therapy alone as compared to control as assessed by tumor volume over time. FIG. 5C: effect of bosentan plus antibody therapy or antibody therapy alone in mouse model as assessed by elastic modulus.

FIGS. 6A-6B shows mean transit time (FIG. 6A) and rise time (FIG. 6B) calculated from the time intensity curves that were produced during the dynamic contrast enhanced ultrasound measurements of mice bearing 4T1 tumors. Anti-PD-1/anti-CTLA-4 (ICB) and bosentan plus ICB (Bos+ICB) were compared with control.

FIGS. 7A-7B show measurements of the effect of ketotifen monotherapy in mice implanted with MCA205 tumors (FIG. 7A) or K7M2wt tumors (FIG. 7B). All data are expressed as mean+/−standard error of the mean (n=5-7 mice per treatment group).

FIG. 8 shows IFP levels in untreated mice and daily ketotifen treated mice bearing MCA205 tumors for 7 days (n=7 mice per treatment group).

FIG. 9 shows longitudinal measurements of tissue-level macroscopic Young's modulus of MCA205 tumors in mice after treatment with the indicated dose of ketotifen or control.

FIGS. 10A-10D is a series of graphs showing the effect of ketotifen on vascular perfusion or functional perfusion area in mice bearing MCA205 or K7M2 wt tumors. FIG. 10A and FIG. 10B show the effects on MCA205 tumors. FIGS. 10C and 10D show the effects on K7M2 wt tumors.

FIGS. 11A-11B show the effect of the indicated monotherapies and combination therapies on mice bearing MCA205 tumors (FIG. 11A) or K7M2 wt (FIG. 11B) tumors.

FIGS. 12A-12B shows a schematic for treatment with tranilast with an anti-PD-L1 antibody in mice bearing MCA205 tumors (FIG. 12A) or E0771 tumors (FIG. 12B).

FIG. 13 shows the results of treatment of mice bearing MCA205 tumors with control, anti-PD-L1 antibody, or the indicated concentrations of tranilast pretreatment with anti-PD-L1 therapy.

FIG. 14 shows the results of treatment of mice bearing E0771 tumors with control, anti-PD-L1 antibody, or the indicated concentrations of tranilast pretreatment with anti-PD-L1 therapy.

FIGS. 15A-15E is a series of graphs showing correlations between elastic modulus and relative tumor volume (FIG. 15A), mean transit time and relative tumor volume (FIG. 15B), rise time and relative tumor volume (15C), elastic modulus and mean transit time (FIG. 15D), and elastic modulus and rise time (FIG. 15E) for mice treated with bosentan or tranilast or mice pretreated with bosentan or tranilast and then treated with immunotherapy. The correlations are assessed by measurements at the initiation of immunotherapy and the end of experiment.

FIGS. 16A-16E is a series of graphs showing correlations between elastic modulus and relative tumor volume (FIG. 16A), wash in slope and relative tumor volume (FIG. 16B), time to peak and relative tumor volume (FIG. 16C), elastic modulus and wash in slope (FIG. 16D), and elastic modulus and time to peak (FIG. 16E) for mice bearing MCA205 tumors treated with control, anti-PD-L1 alone, or tranilast pretreatment followed by immunotherapy with anti-PD-L1.

FIG. 17 shows the results of treatment of mice bearing MCA205 tumors with the indicated therapies.

FIGS. 18A-18E is a series of graphs showing correlations between elastic modulus and relative tumor volume (FIG. 18A), wash in slope and relative tumor volume (FIG. 18B), time to peak and relative tumor volume (FIG. 18C), elastic modulus and wash in slope (FIG. 18D), and elastic modulus and time to peak (FIG. 18E) for mice bearing E0771 tumors treated with control, anti-PD-L1 alone, or tranilast pretreatment followed by immunotherapy with anti-PD-L1.

FIG. 19 shows the results of treatment of mice bearing E0771 tumors.

FIGS. 20A-20E is a series of graphs showing correlations between elastic modulus and relative tumor volume (FIG. 20A), wash in slope and relative tumor volume (FIG. 20B), time to peak and relative tumor volume (FIG. 20C), elastic modulus and time to peak (FIG. 20D), and elastic modulus and wash in slope (FIG. 20E) for mice bearing MCA205 or E0771 tumors treated with control, anti-PD-L1 alone, or tranilast pretreatment followed by immunotherapy with anti-PD-L1.

### DETAILED DESCRIPTION

### I. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

Compositions or methods “comprising” one or more recited elements may include other elements not specifically recited. For example, a composition that comprises antibody may contain the antibody alone or in combination with other ingredients.

It is understood that aspects and embodiments of the invention described herein include “comprising,” “consisting,” and “consisting essentially of” aspects and embodiments.

Designation of a range of values includes all integers within or defining the range.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The term “weight-based dose”, as referred to herein, means that a dose administered to a subject is calculated based on the weight of the subject. For example, when a subject with 60 kg body weight requires 2.0 mg/kg of bosentan or a checkpoint inhibitor, one can calculate and use the appropriate amount of the bosentan or checkpoint inhibitor (i.e., 120 mg) for administration to said subject.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., bosentan and/or checkpoint inhibitor). For example, a subject with 60 kg body weight and a subject with 100 kg body weight would receive the same dose of bosentan or checkpoint inhibitor.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a breast cancer refers to a tumor that is the result of a metastasized breast cancer.

“Administering” or “administration” refer to the physical introduction of a therapeutic agent to a subject, using any of the various methods and delivery systems known to those skilled in the art. Exemplary routes of administration for the bosentan and/or checkpoint inhibitor include enteral routes of administration and intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

In antibodies or other proteins described herein, reference to amino acid residues corresponding to those specified by SEQ ID NO includes post-translational modifications of such residues.

The term “antibody” denotes immunoglobulin proteins produced by the body in response to the presence of an antigen and that bind to the antigen, as well as antigen-binding fragments and engineered variants thereof. Hence, the term “antibody” includes, for example, intact monoclonal antibodies (e.g., antibodies produced using hybridoma technology) and antigen-binding antibody fragments, such as a F(ab′)₂, a Fv fragment, a diabody, a single-chain antibody, an scFv fragment, or an scFv-Fc. Genetically, engineered intact antibodies and fragments such as chimeric antibodies, humanized antibodies, single-chain Fv fragments, single-chain antibodies, diabodies, minibodies, linear antibodies, multivalent or multi-specific (e.g., bispecific) hybrid antibodies, and the like, are also included. Thus, the term “antibody” is used expansively to include any protein that comprises an antigen-binding site of an antibody and is capable of specifically binding to its antigen.

The term antibody or antigen-binding fragment thereof includes a “conjugated” antibody or antigen-binding fragment thereof or an “antibody-drug conjugate (ADC)” in which an antibody or antigen-binding fragment thereof is covalently or non-covalently bound to a pharmaceutical agent, e.g., to a cytostatic or cytotoxic drug.

The term “chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

An “antigen-binding site of an antibody” is that portion of an antibody that is sufficient to bind to its antigen. The minimum such region is typically a variable domain or a genetically engineered variant thereof. Single domain binding sites can be generated from camelid antibodies (see Muyldermans and Lauwereys, Mol. Recog. 12: 131-140, 1999; Nguyen et al., EMBO J. 19:921-930, 2000) or from VH domains of other species to produce single-domain antibodies (“dAbs,” see Ward et al., Nature 341: 544-546, 1989; U.S. Pat. No. 6,248,516 to Winter et al). Commonly, an antigen-binding site of an antibody comprises both a heavy chain variable (VH) domain and a light chain variable (VL) domain that bind to a common epitope. Within the context of the present invention, an antibody may include one or more components in addition to an antigen-binding site, such as, for example, a second antigen-binding site of an antibody (which may bind to the same or a different epitope or to the same or a different antigen), a peptide linker, an immunoglobulin constant region, an immunoglobulin hinge, an amphipathic helix (see Pack and Pluckthun, Biochem. 31: 1579-1584, 1992), a non-peptide linker, an oligonucleotide (see Chaudri et al., FEBS Letters 450:23-26, 1999), a cytostatic or cytotoxic drug, and the like, and may be a monomeric or multimeric protein. Examples of molecules comprising an antigen-binding site of an antibody are known in the art and include, for example, Fv, single-chain Fv (scFv), Fab, Fab′, F(ab′)2, F(ab)c, diabodies, minibodies, nanobodies, Fab-scFv fusions, bispecific (scFv)4-IgG, and bispecific (scFv)2-Fab. (See, e.g., Hu et al, Cancer Res. 56:3055-3061, 1996; Atwell et al., Molecular Immunology 33: 1301-1312, 1996; Carter and Merchant, Curr. Op. Biotechnol. 8:449-454, 1997; Zuo et al., Protein Engineering 13:361-367, 2000; and Lu et al., J. Immunol. Methods 267:213-226, 2002.)

The term “immunoglobulin” refers to a protein consisting of one or more polypeptides substantially encoded by immunoglobulin gene(s). One form of immunoglobulin constitutes the basic structural unit of native (i.e., natural or parental) antibodies in vertebrates. This form is a tetramer and consists of two identical pairs of immunoglobulin chains, each pair having one light chain and one heavy chain. In each pair, the light and heavy chain variable regions (VL and VH) are together primarily responsible for binding to an antigen, and the constant regions are primarily responsible for the antibody effector functions. Five classes of immunoglobulin protein (IgG, IgA, IgM, IgD, and IgE) have been identified in higher vertebrates. IgG comprises the major class, and it normally exists as the second most abundant protein found in plasma. In humans, IgG consists of four subclasses, designated IgG1, IgG2, IgG3, and IgG4. Each immunoglobulin heavy chain possesses a constant region that consists of constant region protein domains (CH1, hinge, CH2, and CH3; IgG3 also contains a CH4 domain) that are essentially invariant for a given subclass in a species.

DNA sequences encoding human and non-human immunoglobulin chains are known in the art. (See, e.g., Ellison et al, DNA 1: 11-18, 1981; Ellison et al, Nucleic Acids Res. 10:4071-4079, 1982; Kenten et al., Proc. Natl. Acad. Set USA 79:6661-6665, 1982; Seno et al., Nucl. Acids Res. 11:719-726, 1983; Riechmann et al., Nature 332:323-327, 1988; Amster et al., Nucl. Acids Res. 8:2055-2065, 1980; Rusconi and Kohler, Nature 314:330-334, 1985; Boss et al., Nucl. Acids Res. 12:3791-3806, 1984; Bothwell et al., Nature 298:380-382, 1982; van der Loo et al., Immunogenetics 42:333-341, 1995; Karlin et al., J. Mol. Evol. 22: 195-208, 1985; Kindsvogel et al., DNA 1:335-343, 1982; Breiner et al., Gene 18: 165-174, 1982; Kondo et al., Eur. J. Immunol. 23:245-249, 1993; and GenBank Accession No. J00228.) For a review of immunoglobulin structure and function see Putnam, The Plasma Proteins, Vol V, Academic Press, Inc., 49-140, 1987; and Padlan, Mol. Immunol. 31: 169-217, 1994. The term “immunoglobulin” is used herein for its common meaning, denoting an intact antibody, its component chains, or fragments of chains, depending on the context.

Full-length immunoglobulin “light chains” (about 25 kDa or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 kDa or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin light or heavy chain variable region (also referred to herein as a “light chain variable domain” (“VL domain”) or “heavy chain variable domain” (“VH domain”), respectively) consists of a “framework” region interrupted by three “complementarity determining regions” or “CDRs.” The framework regions serve to align the CDRs for specific binding to an epitope of an antigen. Thus, the term “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4.

The assignment of amino acids to each variable region domain is in accordance with the definitions of Kabat, Sequences of Proteins of Immunological Interest (National Institutes of Health, Bethesda, M D, 1987 and 1991). Kabat also provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as CDR-L1, CDR-L2 and CDR-L3. CDRs 1, 2 and 3 of a VH domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat.

Numbering of the heavy chain constant region is via the EU index as set forth in Kabat (Kabat, Sequences of Proteins of Immunological Interest, National Institutes of Health, Bethesda, M D, 1987 and 1991).

Unless the context dictates otherwise, the term “monoclonal antibody” is not limited to antibodies produced through hybridoma technology. The term “monoclonal antibody” can include an antibody that is derived from a single clone, including any eukaryotic, prokaryotic or phage clone. In particular embodiments, the antibodies described herein are monoclonal antibodies.

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

The term “humanized VH domain” or “humanized VL domain” refers to an immunoglobulin VH or VL domain comprising some or all CDRs entirely or substantially from a non-human donor immunoglobulin (e.g., a mouse or rat) and variable domain framework sequences entirely or substantially from human immunoglobulin sequences. The non-human immunoglobulin providing the CDRs is called the “donor” and the human immunoglobulin providing the framework is called the “acceptor.” In some instances, humanized antibodies will retain some non-human residues within the human variable domain framework regions to enhance proper binding characteristics (e.g., mutations in the frameworks may be required to preserve binding affinity when an antibody is humanized).

A “humanized antibody” is an antibody comprising one or both of a humanized VH domain and a humanized VL domain. Immunoglobulin constant region(s) need not be present, but if they are, they are entirely or substantially from human immunoglobulin constant regions.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence.

Human acceptor sequences can be selected for a high degree of sequence identity in the variable region frameworks with donor sequences to match canonical forms between acceptor and donor CDRs among other criteria. Thus, a humanized antibody is an antibody having CDRs entirely or substantially from a donor antibody and variable region framework sequences and constant regions, if present, entirely or substantially from human antibody sequences. Similarly, a humanized heavy chain typically has all three CDRs entirely or substantially from a donor antibody heavy chain, and a heavy chain variable region framework sequence and heavy chain constant region, if present, substantially from human heavy chain variable region framework and constant region sequences. Similarly, a humanized light chain typically has all three CDRs entirely or substantially from a donor antibody light chain, and a light chain variable region framework sequence and light chain constant region, if present, substantially from human light chain variable region framework and constant region sequences.

A CDR in a humanized antibody is substantially from a corresponding CDR in a non-human antibody when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering), or wherein about 100% of corresponding residues (as defined by Kabat numbering), are identical between the respective CDRs. The variable region framework sequences of an antibody chain or the constant region of an antibody chain are substantially from a human variable region framework sequence or human constant region respectively when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical.

Although humanized antibodies often incorporate all six CDRs (preferably as defined by Kabat or IMGT®) from a mouse antibody, they can also be made with fewer than all six CDRs (e.g., at least 3, 4, or 5) CDRs from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164: 1432-1441, 2000).

A CDR in a humanized antibody is “substantially from” a corresponding CDR in a non-human antibody when at least 60%, at least 85%, at least 90%, at least 95% or 100% of corresponding residues (as defined by Kabat (or IMGT)) are identical between the respective CDRs. In particular variations of a humanized VH or VL domain in which CDRs are substantially from a non-human immunoglobulin, the CDRs of the humanized VH or VL domain have no more than six (e.g., no more than five, no more than four, no more than three, no more than two, or nor more than one) amino acid substitutions (preferably conservative substitutions) across all three CDRs relative to the corresponding non-human VH or VL CDRs. The variable region framework sequences of an antibody VH or VL domain or, if present, a sequence of an immunoglobulin constant region, are “substantially from” a human VH or VL framework sequence or human constant region, respectively, when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical. Hence, all parts of a humanized antibody, except the CDRs, are typically entirely or substantially from corresponding parts of natural human immunoglobulin sequences.

Antibodies are typically provided in isolated form. This means that an antibody is typically at least about 50% w/w pure of interfering proteins and other contaminants arising from its production or purification but does not exclude the possibility that the antibody is combined with an excess of pharmaceutical acceptable carrier(s) or other vehicle intended to facilitate its use. Sometimes antibodies are at least about 60%, about 70%, about 80%, about 90%, about 95% or about 99% w/w pure of interfering proteins and contaminants from production or purification. Antibodies, including isolated antibodies, can be conjugated to cytotoxic agents and provided as antibody drug conjugates.

Specific binding of an antibody to its target antigen typically refers an affinity of at least about 10⁶, about 10⁷, about 10⁸, about 10⁹, or about 10¹⁰ M⁻¹. Specific binding is detectably higher in magnitude and distinguishable from non-specific binding occurring to at least one non-specific target. Specific binding can be the result of formation of bonds between particular functional groups or particular spatial fit (e.g., lock and key type), whereas nonspecific binding is typically the result of van der Waals forces.

The term “epitope” refers to a site of an antigen to which an antibody binds. An epitope can be formed from contiguous amino acids or noncontiguous amino acids juxtaposed by tertiary folding of one or more proteins. Epitopes formed from contiguous amino acids are typically retained upon exposure to denaturing agents, e.g., solvents, whereas epitopes formed by tertiary folding are typically lost upon treatment with denaturing agents, e.g., solvents. An epitope typically includes at least about 3, and more usually, at least about 5, at least about 6, at least about 7, or about 8-10 amino acids in a unique spatial conformation. Methods of determining spatial conformation of epitopes include, for example, x-ray crystallography and two-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues.

Alternatively, two antibodies have the same epitope if all amino acid mutations in the antigen that reduce or eliminate binding of one antibody reduce or eliminate binding of the other (provided that such mutations do not produce a global alteration in antigen structure). Two antibodies have overlapping epitopes if some amino acid mutations that reduce or eliminate binding of one antibody reduce or eliminate binding of the other antibody.

Competition between antibodies can be determined by an assay in which a test antibody inhibits specific binding of a reference antibody to a common antigen (see, e.g., Junghans et al., Cancer Res. 50: 1495, 1990). A test antibody competes with a reference antibody if an excess of a test antibody inhibits binding of the reference antibody.

Antibodies identified by competition assay (competing antibodies) include antibodies that bind to the same epitope as the reference antibody and antibodies that bind to an adjacent epitope sufficiently proximal to the epitope bound by the reference antibody for steric hindrance to occur. Antibodies identified by a competition assay also include those that indirectly compete with a reference antibody by causing a conformational change in the target protein thereby preventing binding of the reference antibody to a different epitope than that bound by the test antibody.

An antibody effector function refers to a function contributed by an Fc region of an Ig. Such functions can be, for example, antibody-dependent cellular cytotoxicity (ADCC), antibody-dependent cellular phagocytosis (ADCP), or complement-dependent cytotoxicity (CDC). Such function can be affected by, for example, binding of an Fc region to an Fc receptor on an immune cell with phagocytic or lytic activity or by binding of an Fc region to components of the complement system. Typically, the effect(s) mediated by the Fc-binding cells or complement components result in inhibition and/or depletion of the LIV1-targeted cell. Fc regions of antibodies can recruit Fc receptor (FcR)-expressing cells and juxtapose them with antibody-coated target cells. Cells expressing surface FcR for IgGs including FcγRIII (CD16), FcγRII (CD32) and FcγRIII (CD64) can act as effector cells for the destruction of IgG-coated cells. Such effector cells include monocytes, macrophages, natural killer (NK) cells, neutrophils and eosinophils. Engagement of FcγR by IgG activates ADCC or ADCP. ADCC is mediated by CD16+ effector cells through the secretion of membrane pore-forming proteins and proteases, while phagocytosis is mediated by CD32+ and CD64+ effector cells (see Fundamental Immunology, 4^{th} ed., Paul ed., Lippincott-Raven, N.Y., 1997, Chapters 3, 17 and 30; Uchida et al., J. Exp. Med. 199:1659-69, 2004; Akewanlop et al., Cancer Res. 61:4061-65, 2001; Watanabe et al., Breast Cancer Res. Treat. 53: 199-207, 1999).

In addition to ADCC and ADCP, Fc regions of cell-bound antibodies can also activate the complement classical pathway to elicit CDC. C1q of the complement system binds to the Fc regions of antibodies when they are complexed with antigens. Binding of C1q to cell-bound antibodies can initiate a cascade of events involving the proteolytic activation of C4 and C2 to generate the C3 convertase. Cleavage of C3 to C3b by C3 convertase enables the activation of terminal complement components including C5b, C6, C7, C8 and C9. Collectively, these proteins form membrane-attack complex pores on the antibody-coated cells. These pores disrupt the cell membrane integrity, killing the target cell (see Immunobiology, 6^{th} ed., Janeway et al, Garland Science, N. Y., 2005, Chapter 2).

The term “antibody-dependent cellular cytotoxicity” or “ADCC” refers to a mechanism for inducing cell death that depends on the interaction of antibody-coated target cells with immune cells possessing lytic activity (also referred to as effector cells). Such effector cells include natural killer cells, monocytes/macrophages and neutrophils. The effector cells attach to an Fc region of Ig bound to target cells via their antigen-combining sites. Death of the antibody-coated target cell occurs as a result of effector cell activity. In certain exemplary embodiments, an anti-LIV1 IgG1 antibody of the invention mediates equal or increased ADCC relative to a parental antibody and/or relative to an anti-LIV1 IgG3 antibody.

The term “antibody-dependent cellular phagocytosis” or “ADCP” refers to the process by which antibody-coated cells are internalized, either in whole or in part, by phagocytic immune cells (e.g., by macrophages, neutrophils and/or dendritic cells) that bind to an Fc region of Ig. In certain exemplary embodiments, an anti-LIV1 IgG1 antibody of the invention mediates equal or increased ADCP relative to a parental antibody and/or relative to an anti-LIV1 IgG3 antibody.

The term “complement-dependent cytotoxicity” or “CDC” refers to a mechanism for inducing cell death in which an Fc region of a target-bound antibody activates a series of enzymatic reactions culminating in the formation of holes in the target cell membrane.

Typically, antigen-antibody complexes such as those on antibody-coated target cells bind and activate complement component C1q, which in turn activates the complement cascade leading to target cell death. Activation of complement may also result in deposition of complement components on the target cell surface that facilitate ADCC by binding complement receptors (e.g., CR3) on leukocytes.

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. In certain embodiments, a cytotoxic agent is conjugated to an antibody or administered in combination with an antibody. Suitable cytotoxic agents are described further herein.

A “cytostatic effect” refers to the inhibition of cell proliferation. A “cytostatic agent” refers to a compound that has a cytostatic effect on a cell, thereby mediating inhibition of growth and/or expansion of a specific cell type and/or subset of cells. Suitable cytostatic agents are described further herein.

As used herein, “subtherapeutic dose” means a dose of a therapeutic compound (e.g., bosentan or a checkpoint inhibitor) that is lower than the usual or typical dose of the therapeutic compound when administered alone for the treatment of a hyperproliferative disease (e.g., cancer) and/or, for bosentan, that is lower than the usual or typical dose used to treat its indicated disease (i.e. pulmonary hypertension).

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSARg), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®, Bristol-Myers Squibb Oncology, Princeton, N.J.), albumin-engineered nanoparticle formulation of paclitaxel (ABRAXANE™), and docetaxel (TAXOTERE®, Rhome-Poulene Rorer, Antony, France); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMF0); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors; tyrosine kinase inhibitors; serine-threonine kinase inhibitors such as rapamycin (sirolimus, RAPAMUNE®); farnesyltransferase inhibitors such as lonafarnib (SCH 6636, SARASAR™); checkpoint inhibitors (e.g. inhibitors of CTLA-4, PD-1, PD-L1, PD-L2, B7-H3, B7-H4, BMA, HVEM, TIM3, GALS, LAG3, VISTA, KIR, 2B4, CD160, CGEN-15049, CHK1, CHK2, A2aR, or B-7 family ligands); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin, and pharmaceutically acceptable salts, acids or derivatives of any of the above; as well as combinations of two or more of the above.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rlL-2; LURTOTECAN® topoisomerase 1 inhibitor; ABARELIX® rmRH; Vinorelbine and Esperamicins (see U.S. Pat. No. 4,675,187), and pharmaceutically acceptable salts, acids or derivatives of any of the above; as well as combinations of two or more of the above.

The terms “baseline” or “baseline value” used interchangeably herein can refer to a measurement or characterization of a symptom before the administration of the therapy (e.g., bosentan, or a pharmaceutically acceptable salt thereof as described herein and/or a checkpoint inhibitor as described herein) or at the beginning of administration of the therapy. The baseline value can be compared to a reference value in order to determine the reduction or improvement of a symptom of a disease, such as a cancer. The terms “reference” or “reference value” used interchangeably herein can refer to a measurement or characterization of a symptom after administration of the therapy (e.g., bosentan, or a pharmaceutically acceptable salt thereof as described herein and/or a checkpoint inhibitor as described herein). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “objective response rate” or “ORR” refers to the sum of complete response (CR) rate and partial response (PR) rate.

As used herein, “overall survival” or “OS” refers to the percentage of individuals in a group who are likely to be alive after a particular duration of time.

The term “patient” or “subject” includes human and other mammalian subjects such as non-human primates, rabbits, rats, mice, and the like and transgenic species thereof, that receive either prophylactic or therapeutic treatment.

The term “effective amount,” in the context of treatment of a solid tumor by administration of bosentan and/or a checkpoint inhibitor as described herein, refers to an amount of such bosentan and/or checkpoint inhibitor that is sufficient to inhibit the occurrence or ameliorate one or more symptoms of a solid tumor. An effective amount of an antibody is administered in an “effective regimen.” The term “effective regimen” refers to a combination of amount of the bosentan and/or checkpoint inhibitor being administered and dosage frequency adequate to accomplish prophylactic or therapeutic treatment of the disorder (e.g., prophylactic or therapeutic treatment of a solid tumor).

The term “pharmaceutically acceptable” means approved or approvable by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which bosentan or a checkpoint inhibitor is formulated.

The phrase “pharmaceutically acceptable salt,” refers to pharmaceutically acceptable organic or inorganic salts. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p toluenesulfonate, and pamoate (i.e., 1,1′-methylene bis-(2 hydroxy-3-naphthoate) salts. A pharmaceutically acceptable salt may further comprise an additional molecule such as, e.g., an acetate ion, a succinate ion or other counterion. A counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

Solvates in the context of the invention are those forms of the compounds of the invention that form a complex in the solid or liquid state through coordination with solvent molecules. Hydrates are one specific form of solvates, in which the coordination takes place with water. In certain exemplary embodiments, solvates in the context of the present invention are hydrates.

The terms “inhibit” or “inhibition of” means to reduce by a measurable amount, or to prevent entirely. The term inhibition as used herein can refer to an inhibition or reduction of at least about 10%, at least about 15%, at least about 20%, at least about 25%, at least about 30%, at least about 35%, at least about 40%, at least about 50%, at least about 60%, at least about 70%, at least about 80%, at least about 90%, at least about 95%, or at least about 99%.

The terms “treatment” or “treat” refer to slowing, stopping, or reversing the progression of the disease or condition in a patient, as evidenced by a decrease or elimination of a clinical or diagnostic symptom of the disease or condition. Treatment can include, for example, a decrease in the severity of a symptom, the number of symptoms, or frequency of relapse.

The term “prodrug”, as used herein, refers to a compound that is converted into the active form of the compound upon administration in vivo. For example, a prodrug form of an active compound can be, but not limited to, acylated (acetylated or other) and ether derivatives, carboxylic esters or phosphate esters and various salt forms of the active compound. One of ordinary skill in the art will recognize how to readily modify the compound of subject invention to a prodrug form to facilitate delivery of active compound to a targeted site within the host organism or patient. The skilled artisan also will take advantage of favorable pharmacokinetic parameters of the prodrug form, where applicable, in delivering the desired compound to a targeted site within the host organism or patient to maximize the intended effect of the compound in the treatment of cancer.

As used herein, the term “synergy” or “synergistic effect” when used in connection with a description of the efficacy of a combination of agents, means any measured effect of the combination which is greater than the effect predicted from a sum of the effects of the individual agents.

As used herein, the term “additive” or “additive effect” when used in connection with a description of the efficacy of a combination of agents, means any measured effect of the combination which is similar to the effect predicted from a sum of the effects of the individual agents.

The terms “once about every week,” “once about every two weeks,” or any other similar dosing interval terms as used herein mean approximate numbers. “Once about every week” can include every seven days ±one day, i.e., every six days to every eight days. “Once about every two weeks” can include every fourteen days ±two days, i.e., every twelve days to every sixteen days. “Once about every three weeks” can include every twenty-one days ±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Bosentan

The compound 4-tert-butyl-N-[6-(2-hydroxy-ethoxy)-5-(2-methoxy-phenoxy)-2-(pyrimidin-2-yl) pyrimidin-4-yl] benzenesulfonamide, also known as bosentan, is a dual endothelin receptor antagonist with affinity for both endothelin ETA and ETB receptors useful for the treatment or prevention of endothelin-receptor mediated disorders, such as pulmonary arterial hypertension (“PAH”) in individuals with World Health Organization functional Class III or IV primary pulmonary hypertension and pulmonary hypertension secondary to scleroderma or congenital heart disease or human immunodeficiency virus (HIV) patients. Bosentan is described in U.S. Pat. No. 5,292,740.

In some embodiments, bosentan as used herein refers to a compound as described in U.S. Pat. No. 5,292,740. In some embodiments, bosentan as used herein refers to a compound having the formula:

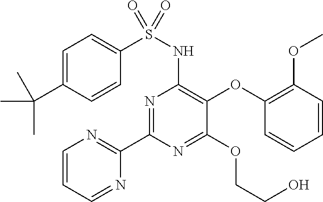 [SMILES: COc1ccccc1Oc1c(NS(=O)(=O)c2ccc(C(C)(C)C)cc2)nc(-c2ncccn2)nc1OCCO]

In some embodiments, provided herein is bosentan hydrate, which has the formula:

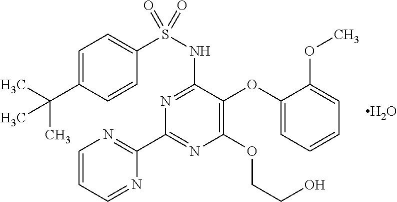

In some embodiments, provided herein is a pharmaceutically acceptable salt of bosentan.

The preparation of bosentan is disclosed in the following patents: European Patent No. 0 526 708, Canadian Patent No. 2,071,193, U.S. Pat. No. 5,292,740, Canadian Patent No. 2,397,258 and U.S. Pat. No. 5,883,254.

### III. Checkpoint Inhibitors

Immune checkpoints refer to inhibitory pathways in the immune system that are responsible for maintaining self-tolerance and modulating the degree of immune system response to minimize peripheral tissue damage. However, tumor cells can also activate immune system checkpoints to decrease the effectiveness of immune response (‘block’ the immune response) against tumor tissues. In contrast to the majority of anti-cancer agents, checkpoint inhibitors do not target tumor cells directly, but rather target lymphocyte receptors or their ligands in order to enhance the endogenous antitumor activity of the immune system. (Pardoll, 2012, Nature Reviews Cancer 12:252-264) Therapy with antagonistic checkpoint blocking antibodies against immune system checkpoints such as CTLA4, PD1 and PD-L1 are one of the most promising new avenues of immunotherapy for cancer and other diseases. Additional checkpoint targets, such as TIM-3, LAG-3, various B-7 ligands, CHK 1 and CHK2 kinases, BTLA, A2aR, and others, are also under investigation. Checkpoint inhibitors include atezolizumab (Tecentriq®), a PD-L1 inhibitor, ipilimumab (Yervoy®), a CTLA-4 inhibitor, and pembrolizumab (Keytruda®) and nivolumab (Opdivo®), both PD-1 inhibitors.

Recent data suggest a secondary mechanism of anti-CTLA-4 antibodies, which could occur within the tumor itself. CTLA-4 has been found to be expressed in tumors at higher levels on regulatory T-cells (also referred to herein as “Treg cells”) as compared with intra-tumoral effector T-cells (also referred to herein as “Teff cells”), resulting in the hypothesis of anti-CTLA-4 preferentially impacting the Treg cell.

One mechanism by which the checkpoint blockade anti-CTLA-4 antibodies mediate anti-tumor effect is by decreasing regulatory T-cells. Due to the distinct mechanism of action of anti-CTLA-4 antibodies, they can successfully combine with the anti-PD-1 checkpoint blockade antibodies which work to release the suppressive signaling conferred to effector T-cells. Dual blockade with these antibodies combine to improve anti-tumor response both preclinically (Proc Natl Acad Sci USA 2010, 107, 4275-4280) and in the clinic (N Engl J Med 2013, 369, 122-133; N Engl J Med 2015, 372, 2006-2017).

In some embodiments, the checkpoint inhibitor inhibits a checkpoint protein selected from the group consisting of CTLA-4, PD-1, PD-L1, PD-L2, B7-H3, B7-H4, BMA, HVEM, TIM3, GAL9, LAG3, VISTA, KIR, 2B4, CD160, CGEN-15049, CHK1, CHK2, A2aR, and B-7 family ligands or a combination thereof. In some embodiments, the checkpoint inhibitor inhibits the checkpoint protein CTLA-4. In some embodiments, the checkpoint inhibitor inhibits the checkpoint protein PD-1. In some embodiments, the checkpoint inhibitor inhibits the checkpoint protein PD-L1. In some embodiments, the checkpoint inhibitor inhibits the checkpoint protein PD-L2. In some embodiments, the checkpoint inhibitor inhibits the checkpoint protein B7-H3. In some embodiments, the checkpoint inhibitor inhibits the checkpoint protein B7-H4. In some embodiments, the checkpoint inhibitor inhibits the checkpoint protein BMA. In some embodiments, the checkpoint inhibitor inhibits the checkpoint protein HVEM. In some embodiments, the checkpoint inhibitor inhibits the checkpoint protein TIM3. In some embodiments, the checkpoint inhibitor inhibits the checkpoint protein GAL9. In some embodiments, the checkpoint inhibitor inhibits the checkpoint protein LAG3. In some embodiments, the checkpoint inhibitor inhibits the checkpoint protein VISTA. In some embodiments, the checkpoint inhibitor inhibits the checkpoint protein KIR. In some embodiments, the checkpoint inhibitor inhibits the checkpoint protein 2B4. In some embodiments, the checkpoint inhibitor inhibits the checkpoint protein CD160. In some embodiments, the checkpoint inhibitor inhibits the checkpoint protein CGEN-15049. In some embodiments, the checkpoint inhibitor inhibits the checkpoint protein CHK1. In some embodiments, the checkpoint inhibitor inhibits the checkpoint protein CHK2. In some embodiments, the checkpoint inhibitor inhibits the checkpoint protein A2aR. In some embodiments, the checkpoint inhibitor inhibits B-7 family ligands. In some embodiments, the checkpoint is an antibody. In some embodiments, the checkpoint inhibitor is an anti-CTLA4 antibody. In some embodiments, the checkpoint inhibitor is an anti-PD-1 antibody. In some embodiments, the checkpoint inhibitor is an anti-PD-L1 antibody. In some embodiments, the checkpoint inhibitor is an anti-PD-L2 antibody. In some embodiments, the checkpoint inhibitor is an anti-B7-H3 antibody. In some embodiments, the checkpoint inhibitor is an anti-B7-H4 antibody. In some embodiments, the checkpoint inhibitor is an anti-BMA antibody. In some embodiments, the checkpoint inhibitor is an anti-HVEM antibody. In some embodiments, the checkpoint inhibitor is an anti-TIM3 antibody. In some embodiments, the checkpoint inhibitor is an anti-GAL9 antibody. In some embodiments, the checkpoint inhibitor is an anti-LAG3 antibody. In some embodiments, the checkpoint inhibitor is an anti-VISTA antibody. In some embodiments, the checkpoint inhibitor is an anti-KIR antibody. In some embodiments, the checkpoint inhibitor is an anti-2B4 antibody. In some embodiments, the checkpoint inhibitor is an anti-CD160 antibody. In some embodiments, the checkpoint inhibitor is an anti-CGEN-15049 antibody. In some embodiments, the checkpoint inhibitor is an anti-CHK1 antibody. In some embodiments, the checkpoint inhibitor is an anti-CHK2 antibody. In some embodiments, the checkpoint inhibitor is an anti-A2aR antibody. In some embodiments, the checkpoint inhibitor is an anti-B7 family ligand antibody. In some embodiments, the checkpoint inhibitor described herein is a monoclonal antibody. In some embodiments, the checkpoint inhibitor described herein is a human antibody. In some embodiments, the checkpoint inhibitor described herein is a humanized antibody. In some embodiments, the checkpoint inhibitor described herein is a chimeric antibody. In some embodiments, the checkpoint inhibitor described herein is a full-length antibody. In some embodiments, the checkpoint inhibitor described herein is an antigen-binding fragment of an antibody. In some embodiments, the antigen-binding fragment is selected from the group consisting of Fab, Fab′ and F(ab′)2, Fd, single-chain Fvs (scFv), single-chain antibodies, disulfide-linked Fvs (sdFv) and fragments comprising either a VL or VH domain. In some embodiments, the checkpoint inhibitor described herein is antibody comprising the complementarity-determining regions (CDRs) of an antibody selected from the group consisting of MEDI0680, AMP-224, nivolumab, pembrolizumab, pidilizumab, MEDI4736, atezolizumab, ipilimumab, tremelimumab, and BMS-936559. In some embodiments, the CDRs are the Kabat CDRs. Kabat et al. (1991), “Sequences of Proteins of Immunological Interest,” 5th Ed. Public Health Service, National Institutes of Health, Bethesda, MD (“Kabat” numbering scheme). In some embodiments, the checkpoint inhibitor described herein comprises the heavy chain variable region and/or the light chain variable region of an antibody selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor described herein comprises the heavy chain variable region of an antibody selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor described herein comprises the light chain variable region of an antibody selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor described herein comprises the heavy chain variable region and the light chain variable region of an antibody selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor described herein is an antibody selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor described herein is a biosimilar of an antibody selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor described herein is MEDI0680. In some embodiments, the checkpoint inhibitor described herein is AMP-224. In some embodiments, the checkpoint inhibitor described herein is nivolumab. In some embodiments, the checkpoint inhibitor described herein is pembrolizumab. In some embodiments, the checkpoint inhibitor described herein is pidilizumab. In some embodiments, the checkpoint inhibitor described herein is MEDI4736. In some embodiments, the checkpoint inhibitor described herein is atezolizumab. In some embodiments, the checkpoint inhibitor described herein is ipilimumab. In some embodiments, the checkpoint inhibitor described herein is tremelimumab. In some embodiments, the checkpoint inhibitor described herein is BMS-936559. In some embodiments, the checkpoint inhibitor is a combination of an anti-PD-1 antibody and an anti-CTLA4 antibody. In some embodiments, the checkpoint inhibitor is a combination of nivolumab and ipilimumab. In some embodiments, the checkpoint inhibitor is a combination of pembrolizumab and ipilimumab. In some embodiments, the checkpoint inhibitor is a combination of an anti-PD-L1 antibody and an anti-CTLA4 antibody. In some embodiments, the checkpoint inhibitor is a combination of atezolizumab and ipilimumab.

### IV. Methods

### A. Treatment of Solid Tumors

In one aspect the invention provides a method for treating a solid tumor in a subject in need thereof comprising administering to the subject an effective amount of bosentan, or a pharmaceutically acceptable salt thereof, in combination with a checkpoint inhibitor. In another aspect, the invention provides a method for initiating, enhancing or prolonging the effects of a checkpoint inhibitor, or enabling a subject to respond to a checkpoint inhibitor in a subject in need thereof comprising administering to the subject an effective amount of bosentan, or a pharmaceutically acceptable salt thereof, in combination with a checkpoint inhibitor, wherein the subject has a solid tumor. In another aspect, the invention provides a method for potentiating the effects of a checkpoint inhibitor in a subject in need thereof comprising administering to the subject an effective amount of bosentan, or a pharmaceutically acceptable salt thereof, in combination with a checkpoint inhibitor, wherein the subject has a solid tumor. In another aspect, the invention provides a method of increasing blood flow of a solid tumor in a subject comprising administering to the subject an effective amount of bosentan, or a pharmaceutically acceptable salt thereof, in combination with a checkpoint inhibitor, wherein increasing blood flow of the solid tumor enhances the effect of the checkpoint inhibitor. In some embodiments, the blood flow of the solid tumor is determined using ultrasound-based blood flow measurements or using histological techniques to measure hypoxia. In some embodiments, the blood flow of the solid tumor is determined using ultrasound-based blood flow measurements. In some embodiments, the blood flow of the solid tumor is determined using histological techniques to measure hypoxia. In some embodiments, blood flow is measured using histological techniques to measure hypoxia in a biopsy from the solid tumor. In another aspect, the invention provides a method of improving the delivery or efficacy of a checkpoint inhibitor in a subject comprising administering an effective amount of bosentan, or a pharmaceutically acceptable salt thereof, in combination with the checkpoint inhibitor, wherein the subject has a solid tumor, thereby improving the delivery or efficacy of the therapy in the subject. In some embodiments, the subject is a human.

In another aspect, the invention provides a method of determining an effective amount of an agent that decompresses blood vessels in a subject with a solid tumor comprising: (a) measuring the blood flow and/or stiffness of the solid tumor; (b) administering to the subject an effective amount of an agent that decompresses blood vessels; and (c) measuring the blood flow and/or stiffness of the solid tumor after administering the agent that decompresses blood vessels, wherein an increase in blood flow and/or a decrease in stiffness following administration of the agent that decompresses blood vessels to the subject indicates that the amount administered was an effective amount. In another aspect, the invention provides a method for treating a solid tumor in a subject in need thereof comprising: (a) measuring the blood flow and/or stiffness of the solid tumor; (b) administering to the subject an effective amount of an agent that decompresses blood vessels; (c) measuring the blood flow and/or stiffness of the solid tumor after administering the agent that decompresses blood vessels; and (d) administering a chemotherapeutic agent if the blood flow of the solid tumor is increased and/or the stiffness of the solid tumor is decreased after administering the agent that decompresses blood vessels. In another aspect, the invention provides a method for treating a solid tumor in a subject in need thereof comprising: (a) measuring the blood flow and/or stiffness of the solid tumor; (b) administering to the subject an effective amount of an agent that decompresses blood vessel; (c) measuring the blood flow and/or stiffness of the solid tumor after administering the agent that decompresses blood vessels; (d) determining that the subject is responsive to a chemotherapeutic agent based on an increase in the blood flow of the solid tumor or a decrease in the stiffness of the solid tumor after administering the agent that decompresses blood vessels; and (e) administering the chemotherapeutic agent to the subject who has been determined to be responsive to the chemotherapeutic agent based on the increase in the blood flow of the solid tumor or the decrease in the stiffness of the solid tumor after administering the agent that decompresses blood vessels. In another aspect, the invention provides a method for predicting response to treatment with a chemotherapeutic agent comprising: (a) measuring the blood flow and/or stiffness of the solid tumor; (b) administering to the subject an effective amount of an agent that decompresses blood vessels; (c) measuring the blood flow and/or stiffness of the solid tumor after administering the agent that decompresses blood vessels, wherein an increase in the blood flow of the solid tumor or a decrease in the stiffness of the solid tumor after administering the agent that decompresses blood vessels indicates that the subject is likely to respond to treatment with the chemotherapeutic agent. In some embodiments, the effective amount of an agent that decompresses blood vessels is determined by measuring the change in blood flow and/or stiffness of the solid tumor following administration of the agent that decompresses blood vessels to the subject, wherein an increase in blood flow and/or a decrease in stiffness following administration of the agent that decompresses blood vessels to the subject indicates that the amount administered was an effective amount. In some embodiments, the method comprises measuring the blood flow of the solid tumor and the blood flow of the solid tumor is increased after administering the agent that decompresses blood vessels. In some embodiments, the method comprises measuring the stiffness of the solid tumor and the stiffness of the solid tumor is decreased after administering the agent that decompresses blood vessels. In some embodiments, the agent that decompresses blood vessels is administered for at least 1 day, at least 2 days, at least 3 days, at least 4 days, or at least 5 days prior to the administration of the chemotherapeutic agent. In some embodiments, the agent that decompresses blood vessels is administered at a dose that increases the blood flow of the solid tumor and/or decreases the stiffness of the solid tumor. In some embodiments, the agent that decompresses blood vessels is selected from the group consisting of an inhibitor of ketotifen, endothelin ETA receptor, an inhibitor of endothelin ETB receptor, an inhibitor of both endothelin ETA and ETB receptors, an angiotensin inhibitor, a glucocorticoid steroid (such as dexamethasone), a vitamin D receptor agonist (such as paricalcitol), tranilast, pirfenidone, a CXCR4 inhibitor (such as plerixafor), metformin and a taxane. In some embodiments, the agent that decompresses blood vessels is an inhibitor of endothelin ETA receptor. In some embodiments, the agent that decompresses blood vessels is an inhibitor of endothelin ETB receptor. In some embodiments, the agent that decompresses blood vessels is an inhibitor of both endothelin ETA and endothelin ETB receptors. In some embodiments, the agent that decompresses blood vessels is an angiotensin inhibitor. In some embodiments, the agent that decompresses blood vessels is dexamethasone. In some embodiments, the agent that decompresses blood vessels is a glucocorticoid inhibitor. In some embodiments, the agent that decompresses blood vessels is a vitamin D receptor agonist. In some embodiments, the agent that decompresses blood vessels is paricalcitol. In some embodiments, the agent that decompresses blood vessels is tranilast. In some embodiments, the agent that decompresses blood vessels is ketotifen. In some embodiments, the agent that decompresses blood vessels is pirfenidone. In some embodiments, the agent that decompresses blood vessels is a CXCR4 inhibitor. In some embodiments, the agent that decompresses blood vessels is plerixafor. In some embodiments, the agent that decompresses blood vessels is metformin. In some embodiments, the agent that decompresses blood vessels is a taxane. In some embodiments, the agent that decompresses blood vessels is bosentan, or a pharmaceutically acceptable salt thereof. In some embodiments, the agent that decompresses blood vessels is losartan, or a pharmaceutically acceptable salt thereof. In some embodiments, blood flow and/or stiffness of the solid tumor is measured using ultrasound. In some embodiments, blood flow of the solid tumor is measured using histological techniques to measure hypoxia. In some embodiments, the chemotherapeutic agent is a checkpoint inhibitor. In some embodiments, the subject is a human.

In some embodiments of any of the aspects provided herein, administering bosentan, or pharmaceutically acceptable salt thereof, increases the number of anti-tumor T cells that colocalize with the solid tumor. In some embodiments, the number of anti-tumor T cells that colocalize with the solid tumor is increased by at least 10%. In some embodiments, the number of anti-tumor T cells that colocalize with the solid tumor is increased by at least 25%. In some embodiments, the number of anti-tumor T cells that colocalize with the solid tumor is increased by at least 50%. In some embodiments, the number of anti-tumor T cells that colocalize with the solid tumor is increased by at least 100%. In some embodiments, the number of anti-tumor T cells that colocalize with the solid tumor is increased by at least 150%.

In some embodiments of any of the aspects provided herein, administering an agent that decompresses blood vessels reduces the tissue stiffness of the solid tumor. In some embodiments of any of the aspects provided herein, administering bosentan, or pharmaceutically acceptable salt thereof, reduces the tissue stiffness of the solid tumor. In some embodiments, tissue stiffness of the solid tumor is reduced by at least 10%. In some embodiments, tissue stiffness of the solid tumor is reduced by at least 20%. In some embodiments, tissue stiffness of the solid tumor is reduced by at least 25%. In some embodiments, tissue stiffness of the solid tumor is reduced by at least 30%. In some embodiments, tissue stiffness of the solid tumor is reduced by at least 40%. In some embodiments, tissue stiffness of the solid tumor is reduced by at least 50%. In some embodiments, tissue stiffness of the solid tumor is reduced by at least 60%. In some embodiments, tissue stiffness of the solid tumor is reduced by at least 70%. In some embodiments, tissue stiffness of the solid tumor is reduced by at least 75%. In some embodiments, tissue stiffness of the solid tumor is measured using ultrasound elastography.

In some embodiments of any of the aspects provided herein, administering an agent that decompresses blood vessels decreases the levels of an extracellular matrix protein in the solid tumor. In some embodiments of any of the aspects provided herein, administering bosentan, or pharmaceutically acceptable salt thereof, decreases the levels of an extracellular matrix protein in the solid tumor. In some embodiments, the levels of an extracellular matrix protein in the solid tumor are reduced by at least 10%. In some embodiments, the levels of an extracellular matrix protein in the solid tumor are reduced by at least 20%. In some embodiments, the levels of an extracellular matrix protein in the solid tumor are reduced by at least 25%. In some embodiments, the levels of an extracellular matrix protein in the solid tumor are reduced by at least 30%. In some embodiments, the levels of an extracellular matrix protein in the solid tumor are reduced by at least 40%. In some embodiments, the levels of an extracellular matrix protein in the solid tumor are reduced by at least 50%. In some embodiments, the levels of an extracellular matrix protein in the solid tumor are reduced by at least 60%. In some embodiments, the levels of an extracellular matrix protein in the solid tumor are reduced by at least 70%. In some embodiments, the levels of an extracellular matrix protein in the solid tumor are reduced by at least 75%. In some embodiments, the extracellular matrix protein is collagen I. In some embodiments, the extracellular matrix protein is hyaluronan binding protein (HABP).

In some embodiments of any of the aspects provided herein, administering an agent that decompresses blood vessels reduces hypoxia in the solid tumor. In some embodiments of any of the aspects provided herein, administering bosentan, or pharmaceutically acceptable salt thereof, reduces hypoxia in the solid tumor. In some embodiments, hypoxia is reduced by at least 10%. In some embodiments, hypoxia is reduced by at least 20%. In some embodiments, hypoxia is reduced by at least 25%. In some embodiments, hypoxia is reduced by at least 30%. In some embodiments, hypoxia is reduced by at least 40%. In some embodiments, hypoxia is reduced by at least 50%. In some embodiments, hypoxia is reduced by at least 60%. In some embodiments, hypoxia is reduced by at least 70%. In some embodiments, hypoxia is reduced by at least 75%.

In some embodiments of any of the aspects provided herein, the solid tumor is selected from the group consisting of breast cancer, breast cancer lung metastases, sarcoma, pancreatic cancer, ovarian cancer, liver metastases, prostate cancer, brain cancer, melanoma, renal cell carcinoma, colorectal cancer, hepatocellular carcinoma, lung cancer, squamous cell carcinoma of the head and neck, urothelial carcinoma, esophageal squamous cell carcinoma, gastric cancer, esophageal cancer, cervical cancer, Merkel cell carcinoma, endometrial carcinoma, mesothelioma, and cutaneous squamous cell carcinoma. In some embodiments, the solid tumor is breast cancer. In some embodiments, the breast cancer has high tumor endothelin-B receptor expression relative to non-tumor tissue. In some embodiments, the breast cancer is triple negative breast cancer. In some embodiments, the solid tumor is a lung metastasis from breast cancer. In some embodiments, the solid tumor is a sarcoma. In some embodiments, the solid tumor is pancreatic cancer. In some embodiments, the solid tumor is ovarian cancer. In some embodiments, the solid tumor is a liver metastasis. In some embodiments, the liver metastasis is from colorectal cancer. In some embodiments, the solid tumor is a prostate cancer. In some embodiments, the prostate cancer has high tumor endothelin-A receptor expression relative to non-tumor tissue. In some embodiments, the solid cancer is a brain cancer. In some embodiments, the brain cancer has high tumor endothelin-A receptor expression relative to non-tumor tissue. In some embodiments, the solid tumor is melanoma. In some embodiments, the solid tumor is renal cell carcinoma. In some embodiments, the solid tumor is colorectal cancer. In some embodiments, the colorectal cancer has high tumor endothelin-A receptor expression relative to non-tumor tissue. In some embodiments, the colorectal cancer has low tumor endothelin-B receptor expression relative to non-tumor tissue. In some embodiments, the colorectal cancer has high tumor endothelin-A receptor expression and low endothelin-B receptor expression relative to non-tumor tissue. In some embodiments, the solid tumor is hepatocellular carcinoma. In some embodiments, the solid tumor is lung cancer. In some embodiments, the lung cancer expresses endothelin-A receptor. In some embodiments, the lung cancer expresses endothelin-B receptor. In some embodiments, the lung cancer expresses both endothelin-A receptor and endothelin-B receptor. In some embodiments, the lung cancer has high tumor endothelin-A receptor expression relative to non-tumor tissue. In some embodiments, the lung cancer has high tumor endothelin-B receptor expression relative to non-tumor tissue. In some embodiments, the lung cancer has high tumor endothelin-A receptor and endothelin-B receptor expression relative to non-tumor tissue. In some embodiments, the lung cancer is non-small cell lung cancer. In some embodiments, the lung cancer is small cell lung cancer. In some embodiments, the solid tumor is squamous cell carcinoma of the head and neck. In some embodiments, the solid tumor is urothelial carcinoma. In some embodiments, the solid tumor is esophageal squamous cell carcinoma. In some embodiments, the solid tumor is gastric cancer. In some embodiments, the solid tumor is esophageal cancer. In some embodiments, the solid tumor is cervical cancer. In some embodiments, the solid tumor is Merkel cell carcinoma. In some embodiments, the solid tumor is endometrial carcinoma. In some embodiments, the solid tumor is mesothelioma. In some embodiments, the solid tumor is cutaneous squamous cell carcinoma. In some embodiments, the solid tumor is a cancer that has compressed blood vessels and/or is hypoperfused. In some embodiments, the solid tumor is a cancer that has compressed blood vessels. In some embodiments, the solid tumor is a cancer that is hypoperfused. In some embodiments, the solid tumor that has compressed blood vessels and/or is hypoperfused is selected from the group consisting of breast cancer, breast cancer lung metastases, pancreatic cancer, ovarian cancer, and liver metastases. In some embodiments, the solid tumor that has compressed blood vessels and/or is hypoperfused is breast cancer. In some embodiments, the breast cancer has high tumor endothelin-B receptor expression relative to non-tumor tissue. In some embodiments, the breast cancer is triple negative breast cancer. In some embodiments, the solid tumor that has compressed blood vessels and/or is hypoperfused is pancreatic cancer. In some embodiments, the solid tumor that has compressed blood vessels and/or is hypoperfused is ovarian cancer. In some embodiments, the solid tumor that has compressed blood vessels and/or is hypoperfused is a liver metastasis. In some embodiments, the liver metastasis is from colorectal cancer. In some embodiments, the solid tumor that has compressed blood vessels and/or is hypoperfused is a lung metastasis. In some embodiments, the liver metastasis is from breast cancer. In some embodiments, the solid tumor is a cancer that has endothelin receptor expression in the tumor vasculature and/or fibroblasts. In some embodiments, the solid tumor is a cancer that has endothelin receptor expression in the tumor vasculature. In some embodiments, the solid tumor is a cancer that has endothelin receptor expression in the tumor fibroblasts. In some embodiments, the solid tumor that has endothelin receptor expression in the tumor vasculature and/or fibroblasts is selected from the group consisting of pancreatic cancer, ovarian cancer, lung cancer, prostate cancer, brain cancer, breast cancer, and colorectal cancer. In some embodiments, the solid tumor that has endothelin receptor expression in the tumor vasculature and/or fibroblasts is pancreatic cancer. In some embodiments, the solid tumor that has endothelin receptor expression in the tumor vasculature and/or fibroblasts is ovarian cancer. In some embodiments, the solid tumor that has endothelin receptor expression in the tumor vasculature and/or fibroblasts is lung cancer. In some embodiments, the lung cancer expresses endothelin-A receptor. In some embodiments, the lung cancer expresses endothelin-B receptor. In some embodiments, the lung cancer expresses both endothelin-A receptor and endothelin-B receptor. In some embodiments, the lung cancer has high tumor endothelin-A receptor expression relative to non-tumor tissue. In some embodiments, the lung cancer has high tumor endothelin-B receptor expression relative to non-tumor tissue. In some embodiments, the lung cancer has high tumor endothelin-A receptor and endothelin-B receptor expression relative to non-tumor tissue. In some embodiments, the lung cancer is non-small cell lung cancer. In some embodiments, the lung cancer is small cell lung cancer. In some embodiments, the solid tumor that has endothelin receptor expression in the tumor vasculature and/or fibroblasts is prostate cancer. In some embodiments, the prostate cancer has high tumor endothelin-A receptor expression relative to non-tumor tissue. In some embodiments, the solid tumor that has endothelin receptor expression in the tumor vasculature and/or fibroblasts is brain cancer. In some embodiments, the brain cancer has high tumor endothelin-A receptor expression relative to non-tumor tissue. In some embodiments, the solid tumor that has endothelin receptor expression in the tumor vasculature and/or fibroblasts is breast cancer. In some embodiments, the breast cancer has high tumor endothelin-B receptor expression relative to non-tumor tissue. In some embodiments, the breast cancer is triple negative breast cancer. In some embodiments, the solid tumor is a lung metastasis from breast cancer. In some embodiments, the solid tumor that has endothelin receptor expression in the tumor vasculature and/or fibroblasts is colorectal cancer. In some embodiments, the colorectal cancer has high tumor endothelin-A receptor expression relative to non-tumor tissue. In some embodiments, the colorectal cancer has low tumor endothelin-B receptor expression relative to non-tumor tissue. In some embodiments, the colorectal cancer has high tumor endothelin-A receptor expression and low endothelin-B receptor expression relative to non-tumor tissue.

### B. Routes of Administration

Chemotherapeutic agents described herein can be administered by any suitable route and mode. Bosentan, or a pharmaceutically acceptable salt thereof, or a checkpoint inhibitor described herein can be administered by any suitable route and mode. Suitable routes of administering compounds or antibodies of the present invention are well known in the art and may be selected by those of ordinary skill in the art. In one embodiment, the bosentan, or pharmaceutically acceptable salt thereof, and/or the checkpoint inhibitor described herein are administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of the chemotherapeutic agent is intraperitoneal injection. In some embodiments, the route of administration of the chemotherapeutic agent is intravenous injection. In some embodiments, the route of administration of bosentan, or pharmaceutically acceptable salt thereof, is intraperitoneal injection. In some embodiments, the route of administration of the checkpoint inhibitor is intraperitoneal injection. In some embodiments, the route of administration of bosentan, or pharmaceutically acceptable salt thereof, is intravenous injection. In some embodiments, the route of administration of the checkpoint inhibitor is intravenous injection. In one embodiment, the bosentan, or pharmaceutically acceptable salt thereof, and/or the checkpoint inhibitor described herein are administered enterally. In some embodiments, the route of administration of bosentan, or pharmaceutically acceptable salt thereof, is enteral. In some embodiments, the route of administration of bosentan, or pharmaceutically acceptable salt thereof, is oral. In some embodiments, the route of administration of the checkpoint inhibitor is enteral. In some embodiments, the route of administration of the checkpoint inhibitor is oral. In some embodiments, the route of administration of the chemotherapeutic agent is enteral. In some embodiments, the route of administration of the chemotherapeutic agent is oral.

### C. Dosage and Frequency of Administration

In one aspect, the present invention provides for methods as described herein comprising administering bosentan, or a pharmaceutically acceptable salt thereof, as described herein and a checkpoint inhibitor as described herein, wherein the subject is administered the bosentan, or a pharmaceutically acceptable salt thereof, as described herein and the checkpoint inhibitor as described herein with particular frequencies. In another aspect, the present invention provides for methods as described herein comprising administering an agent that decompresses blood vessels as described herein and a chemotherapeutic agent as described herein, wherein the subject is administered the agent that decompresses blood vessels as described herein and the chemotherapeutic agent as described herein with particular frequencies.

In one embodiment of the methods or uses or product for uses provided herein an agent that decompresses blood vessels as described herein is administered to the subject in a therapeutically effective amount. In one embodiment of the methods or uses or product for uses provided herein bosentan, or a pharmaceutically acceptable salt thereof, as described herein is administered to the subject in a therapeutically effective amount. In one embodiment of the methods or uses or product for uses provided herein bosentan, or a pharmaceutically acceptable salt thereof, as described herein is administered to the subject at a subtherapeutic dose. In one embodiment of the methods or uses or product for uses provided herein bosentan, or a pharmaceutically acceptable salt thereof, as described herein is administered to the subject at a dose that is sufficient to initiate the effects of a checkpoint inhibitor. In one embodiment of the methods or uses or product for uses provided herein bosentan, or a pharmaceutically acceptable salt thereof, as described herein is administered to the subject at a dose that is sufficient to enhance the effects of a checkpoint inhibitor. In one embodiment of the methods or uses or product for uses provided herein bosentan, or a pharmaceutically acceptable salt thereof, as described herein is administered to the subject at a dose that is sufficient to prolong the effects of a checkpoint inhibitor. In one embodiment of the methods or uses or product for uses provided herein bosentan, or a pharmaceutically acceptable salt thereof, as described herein is administered to the subject at a dose that is sufficient to potentiate the effects of a checkpoint inhibitor. In one embodiment of the methods or uses or product for uses provided herein bosentan, or a pharmaceutically acceptable salt thereof, as described herein is administered to the subject at a dose that is sufficient to improve the delivery of a checkpoint inhibitor to a solid tumor. In one embodiment of the methods or uses or product for uses provided herein bosentan, or a pharmaceutically acceptable salt thereof, as described herein is administered to the subject at a dose that is sufficient to improve the efficacy of a checkpoint inhibitor. In one embodiment of the methods or uses or product for uses provided herein bosentan, or a pharmaceutically acceptable salt thereof, as described herein is administered to the subject at a dose that is sufficient to increase the number of anti-tumor T cells that colocalize with a solid tumor. In one embodiment of the methods or uses or product for uses provided herein bosentan, or a pharmaceutically acceptable salt thereof, as described herein is administered to the subject at a dose that is sufficient to reduce the tissue stiffness of a solid tumor. In one embodiment of the methods or uses or product for uses provided herein bosentan, or a pharmaceutically acceptable salt thereof, as described herein is administered to the subject at a dose that is sufficient to decrease the levels of an extracellular matrix protein in a solid tumor. In one embodiment of the methods or uses or product for uses provided herein bosentan, or a pharmaceutically acceptable salt thereof, as described herein is administered to the subject at a dose that is sufficient to increase blood flow of a solid tumor. In one embodiment of the methods or uses or product for uses provided herein bosentan, or a pharmaceutically acceptable salt thereof, as described herein is administered to the subject at a dose that is sufficient decrease the levels of an extracellular matrix protein in a solid tumor and increase blood flow of the solid tumor. In one embodiment of the methods or uses or product for uses provided herein bosentan, or a pharmaceutically acceptable salt thereof, as described herein is administered to the subject at a dose that is sufficient to reduces hypoxia in a solid tumor.

In some embodiments of the methods or uses or product for uses provided herein an agent that decompresses blood vessels as described herein is administered to the subject at a dose ranging from about 0.01 mg/kg to about 20 mg/kg of the subject's body weight. In some embodiments of the methods or uses or product for uses provided herein bosentan, or a pharmaceutically acceptable salt thereof, as described herein is administered to the subject at a dose ranging from about 0.01 mg/kg to about 20 mg/kg of the subject's body weight. In one embodiment, bosentan, or a pharmaceutically acceptable salt thereof, as described herein is administered at a dose ranging from about 0.05 mg/kg to about 15 mg/kg of the subject's body weight. In one embodiment, bosentan, or a pharmaceutically acceptable salt thereof, as described herein is administered at a dose ranging from about 0.01 mg/kg to about 0.1 mg/kg of the subject's body weight. In one embodiment, bosentan, or a pharmaceutically acceptable salt thereof, as described herein is administered at a dose ranging from about 0.01 mg/kg to about 0.5 mg/kg of the subject's body weight. In one embodiment, bosentan, or a pharmaceutically acceptable salt thereof, as described herein is administered at a dose ranging from about 0.01 mg/kg to about 1.0 mg/kg of the subject's body weight. In one embodiment, bosentan, or a pharmaceutically acceptable salt thereof, as described herein is administered at a dose ranging from about 0.01 mg/kg to about 5 mg/kg of the subject's body weight. In one embodiment, bosentan, or a pharmaceutically acceptable salt thereof, as described herein is administered at a dose ranging from about 0.05 mg/kg to about 0.1 mg/kg of the subject's body weight. In one embodiment, bosentan, or a pharmaceutically acceptable salt thereof, as described herein is administered at a dose ranging from about 0.05 mg/kg to about 10 mg/kg of the subject's body weight. In one embodiment, bosentan, or a pharmaceutically acceptable salt thereof, as described herein is administered at a dose ranging from about 0.05 mg/kg to about 5 mg/kg of the subject's body weight. In one embodiment, bosentan, or a pharmaceutically acceptable salt thereof, as described herein is administered at a dose ranging from about 0.05 mg/kg to about 3 mg/kg of the subject's body weight. In one embodiment, bosentan, or a pharmaceutically acceptable salt thereof, as described herein is administered at a dose ranging from about 0.25 mg/kg to about 10 mg/kg of the subject's body weight. In one embodiment, bosentan, or a pharmaceutically acceptable salt thereof, as described herein is administered at a dose ranging from about 0.25 mg/kg to about 5 mg/kg of the subject's body weight. In one embodiment, bosentan, or a pharmaceutically acceptable salt thereof, as described herein is administered at a dose ranging from about 0.25 mg/kg to about 3 mg/kg of the subject's body weight. In one embodiment, bosentan, or a pharmaceutically acceptable salt thereof, as described herein is administered at a dose ranging from about 0.5 mg/kg to about 10 mg/kg of the subject's body weight. In one embodiment, bosentan, or a pharmaceutically acceptable salt thereof, as described herein is administered at a dose ranging from about 0.5 mg/kg to about 5 mg/kg of the subject's body weight. In one embodiment, bosentan, or a pharmaceutically acceptable salt thereof, as described herein is administered at a dose ranging from about 0.5 mg/kg to about 3 mg/kg of the subject's body weight. In one embodiment, bosentan, or a pharmaceutically acceptable salt thereof, as described herein is administered at a dose ranging from about 0.75 mg/kg to about 10 mg/kg of the subject's body weight. In one embodiment, bosentan, or a pharmaceutically acceptable salt thereof, as described herein is administered at a dose ranging from about 0.75 mg/kg to about 5 mg/kg of the subject's body weight. In one embodiment, bosentan, or a pharmaceutically acceptable salt thereof, as described herein is administered at a dose ranging from about 0.75 mg/kg to about 3 mg/kg of the subject's body weight. In one embodiment, bosentan, or a pharmaceutically acceptable salt thereof, as described herein is administered at a dose ranging from about 1 mg/kg to about 10 mg/kg of the subject's body weight. In one embodiment, bosentan, or a pharmaceutically acceptable salt thereof, as described herein is administered at a dose ranging from about 1 mg/kg to about 5.0 mg/kg of the subject's body weight. In one embodiment, bosentan, or a pharmaceutically acceptable salt thereof, as described herein is administered at a dose ranging from about 1 mg/kg to about 3 mg/kg of the subject's body weight. In one embodiment, bosentan, or a pharmaceutically acceptable salt thereof, as described herein is administered at a dose ranging from about 2 mg/kg to about 20 mg/kg of the subject's body weight. In one embodiment, bosentan, or a pharmaceutically acceptable salt thereof, as described herein is administered at a dose ranging from about 2 mg/kg to about 15 mg/kg of the subject's body weight. In one embodiment, bosentan, or a pharmaceutically acceptable salt thereof, as described herein is administered at a dose ranging from about 2 mg/kg to about 10 mg/kg of the subject's body weight. In one embodiment, bosentan, or a pharmaceutically acceptable salt thereof, as described herein is administered at a dose ranging from about 2 mg/kg to about 5 mg/kg of the subject's body weight. In one embodiment, bosentan, or a pharmaceutically acceptable salt thereof, as described herein is administered at a dose ranging from about 4 mg/kg to about 20 mg/kg of the subject's body weight. In one embodiment, bosentan, or a pharmaceutically acceptable salt thereof, as described herein is administered at a dose ranging from about 4 mg/kg to about 15 mg/kg of the subject's body weight. In one embodiment, bosentan, or a pharmaceutically acceptable salt thereof, as described herein is administered at a dose ranging from about 4 mg/kg to about 10 mg/kg of the subject's body weight. In one embodiment, bosentan, or a pharmaceutically acceptable salt thereof, as described herein is administered at a dose ranging from about 4 mg/kg to about 5 mg/kg of the subject's body weight. In one embodiment, bosentan, or a pharmaceutically acceptable salt thereof, as described herein is administered at a dose ranging from about 5 mg/kg to about 20 mg/kg of the subject's body weight. In one embodiment, bosentan, or a pharmaceutically acceptable salt thereof, as described herein is administered at a dose of about 0.01 mg/kg of the subject's body weight. In one embodiment, bosentan, or a pharmaceutically acceptable salt thereof, as described herein is administered at a dose of about 0.05 mg/kg of the subject's body weight. In one embodiment, bosentan, or a pharmaceutically acceptable salt thereof, as described herein is administered at a dose of about 0.1 mg/kg of the subject's body weight. In one embodiment, bosentan, or a pharmaceutically acceptable salt thereof, as described herein is administered at a dose of about 0.15 mg/kg of the subject's body weight. In one embodiment, bosentan, or a pharmaceutically acceptable salt thereof, as described herein is administered at a dose of about 0.16 mg/kg of the subject's body weight. In one embodiment, bosentan, or a pharmaceutically acceptable salt thereof, as described herein is administered at a dose of about 0.2 mg/kg of the subject's body weight. In one embodiment, bosentan, or a pharmaceutically acceptable salt thereof, as described herein is administered at a dose of about 0.3 mg/kg of the subject's body weight. In one embodiment, bosentan, or a pharmaceutically acceptable salt thereof, as described herein is administered at a dose of about 0.4 mg/kg of the subject's body weight. In one embodiment, bosentan, or a pharmaceutically acceptable salt thereof, as described herein is administered at a dose of about 0.5 mg/kg of the subject's body weight. In one embodiment, bosentan, or a pharmaceutically acceptable salt thereof, as described herein is administered at a dose of about 0.6 mg/kg of the subject's body weight. In one embodiment, bosentan, or a pharmaceutically acceptable salt thereof, as described herein is administered at a dose of about 0.7 mg/kg of the subject's body weight. In one embodiment, bosentan, or a pharmaceutically acceptable salt thereof, as described herein is administered at a dose of about 0.8 mg/kg of the subject's body weight. In one embodiment, bosentan, or a pharmaceutically acceptable salt thereof, as described herein is administered at a dose of about 0.9 mg/kg of the subject's body weight. In one embodiment, bosentan, or a pharmaceutically acceptable salt thereof, as described herein is administered at a dose of about 1 mg/kg of the subject's body weight. In one embodiment, bosentan, or a pharmaceutically acceptable salt thereof, as described herein is administered at a dose of about 1.2 mg/kg of the subject's body weight. In one embodiment, bosentan, or a pharmaceutically acceptable salt thereof, as described herein is administered at a dose of about 1.4 mg/kg of the subject's body weight. In one embodiment, bosentan, or a pharmaceutically acceptable salt thereof, as described herein is administered at a dose of about 1.6 mg/kg of the subject's body weight. In one embodiment, bosentan, or a pharmaceutically acceptable salt thereof, as described herein is administered at a dose of about 1.8 mg/kg of the subject's body weight. In one embodiment, bosentan, or a pharmaceutically acceptable salt thereof, as described herein is administered at a dose of about 2 mg/kg of the subject's body weight. In one embodiment, bosentan, or a pharmaceutically acceptable salt thereof, as described herein is administered at a dose of about 2.2 mg/kg of the subject's body weight. In one embodiment, bosentan, or a pharmaceutically acceptable salt thereof, as described herein is administered at a dose of about 2.4 mg/kg of the subject's body weight. In one embodiment, bosentan, or a pharmaceutically acceptable salt thereof, as described herein is administered at a dose of about 2.6 mg/kg of the subject's body weight. In one embodiment, bosentan, or a pharmaceutically acceptable salt thereof, as described herein is administered at a dose of about 2.8 mg/kg of the subject's body weight. In one embodiment, bosentan, or a pharmaceutically acceptable salt thereof, as described herein is administered at a dose of about 3 mg/kg of the subject's body weight. In one embodiment, bosentan, or a pharmaceutically acceptable salt thereof, as described herein is administered at a dose of about 3.2 mg/kg of the subject's body weight. In one embodiment, bosentan, or a pharmaceutically acceptable salt thereof, as described herein is administered at a dose of about 3.4 mg/kg of the subject's body weight. In one embodiment, bosentan, or a pharmaceutically acceptable salt thereof, as described herein is administered at a dose of about 3.6 mg/kg of the subject's body weight. In one embodiment, bosentan, or a pharmaceutically acceptable salt thereof, as described herein is administered at a dose of about 3.8 mg/kg of the subject's body weight. In one embodiment, bosentan, or a pharmaceutically acceptable salt thereof, as described herein is administered at a dose of about 4 mg/kg of the subject's body weight. In one embodiment, bosentan, or a pharmaceutically acceptable salt thereof, as described herein is administered at a dose of about 4.2 mg/kg of the subject's body weight. In one embodiment, bosentan, or a pharmaceutically acceptable salt thereof, as described herein is administered at a dose of about 4.4 mg/kg of the subject's body weight. In one embodiment, bosentan, or a pharmaceutically acceptable salt thereof, as described herein is administered at a dose of about 4.6 mg/kg of the subject's body weight. In one embodiment, bosentan, or a pharmaceutically acceptable salt thereof, as described herein is administered at a dose of about 4.8 mg/kg of the subject's body weight. In one embodiment, bosentan, or a pharmaceutically acceptable salt thereof, as described herein is administered at a dose of about 5 mg/kg of the subject's body weight. In one embodiment, bosentan, or a pharmaceutically acceptable salt thereof, as described herein is administered at a dose of about 5.2 mg/kg of the subject's body weight. In one embodiment, bosentan, or a pharmaceutically acceptable salt thereof, as described herein is administered at a dose of about 5.4 mg/kg of the subject's body weight. In one embodiment, bosentan, or a pharmaceutically acceptable salt thereof, as described herein is administered at a dose of about 5.6 mg/kg of the subject's body weight. In one embodiment, bosentan, or a pharmaceutically acceptable salt thereof, as described herein is administered at a dose of about 5.8 mg/kg of the subject's body weight. In one embodiment, bosentan, or a pharmaceutically acceptable salt thereof, as described herein is administered at a dose of about 6 mg/kg of the subject's body weight. In one embodiment, bosentan, or a pharmaceutically acceptable salt thereof, as described herein is administered at a dose of about 6.5 mg/kg of the subject's body weight. In one embodiment, bosentan, or a pharmaceutically acceptable salt thereof, as described herein is administered at a dose of about 7 mg/kg of the subject's body weight. In one embodiment, bosentan, or a pharmaceutically acceptable salt thereof, as described herein is administered at a dose of about 7.5 mg/kg of the subject's body weight. In one embodiment, bosentan, or a pharmaceutically acceptable salt thereof, as described herein is administered at a dose of about 8 mg/kg of the subject's body weight. In one embodiment, bosentan, or a pharmaceutically acceptable salt thereof, as described herein is administered at a dose of about 8.5 mg/kg of the subject's body weight. In one embodiment, bosentan, or a pharmaceutically acceptable salt thereof, as described herein is administered at a dose of about 9 mg/kg of the subject's body weight. In one embodiment, bosentan, or a pharmaceutically acceptable salt thereof, as described herein is administered at a dose of about 9.5 mg/kg of the subject's body weight. In one embodiment, bosentan, or a pharmaceutically acceptable salt thereof, as described herein is administered at a dose of about 10 mg/kg of the subject's body weight. In one embodiment, bosentan, or a pharmaceutically acceptable salt thereof, as described herein is administered at a dose of about 11 mg/kg of the subject's body weight. In one embodiment, bosentan, or a pharmaceutically acceptable salt thereof, as described herein is administered at a dose of about 12 mg/kg of the subject's body weight. In one embodiment, bosentan, or a pharmaceutically acceptable salt thereof, as described herein is administered at a dose of about 13 mg/kg of the subject's body weight. In one embodiment, bosentan, or a pharmaceutically acceptable salt thereof, as described herein is administered at a dose of about 14 mg/kg of the subject's body weight. In one embodiment, bosentan, or a pharmaceutically acceptable salt thereof, as described herein is administered at a dose of about 15 mg/kg of the subject's body weight. In one embodiment, bosentan, or a pharmaceutically acceptable salt thereof, as described herein is administered at a dose of about 16 mg/kg of the subject's body weight. In one embodiment, bosentan, or a pharmaceutically acceptable salt thereof, as described herein is administered at a dose of about 17 mg/kg of the subject's body weight. In one embodiment, bosentan, or a pharmaceutically acceptable salt thereof, as described herein is administered at a dose of about 18 mg/kg of the subject's body weight. In one embodiment, bosentan, or a pharmaceutically acceptable salt thereof, as described herein is administered at a dose of about 19 mg/kg of the subject's body weight. In one embodiment, bosentan, or a pharmaceutically acceptable salt thereof, as described herein is administered at a dose of about 20 mg/kg of the subject's body weight.

In some embodiments of the methods or uses or product for uses provided herein an agent that decompresses blood vessels as described herein is administered to the subject at a dose ranging from about 10 mg to about 1250 mg. In some embodiments of the methods or uses or product for uses provided herein bosentan, or a pharmaceutically acceptable salt thereof, as described herein is administered to the subject at a dose ranging from about 10 mg to about 1250 mg. In one embodiment, bosentan, or a pharmaceutically acceptable salt thereof, as described herein is administered at a dose ranging from about 10 mg to about 150 mg. In one embodiment, bosentan, or a pharmaceutically acceptable salt thereof, as described herein is administered at a dose ranging from about 10 mg to about 100 mg. In one embodiment, bosentan, or a pharmaceutically acceptable salt thereof, as described herein is administered at a dose ranging from about 10 mg to about 50 mg. In one embodiment, bosentan, or a pharmaceutically acceptable salt thereof, as described herein is administered at a dose ranging from about 25 mg to about 150 mg. In one embodiment, bosentan, or a pharmaceutically acceptable salt thereof, as described herein is administered at a dose ranging from about 25 mg to about 100 mg. In one embodiment, bosentan, or a pharmaceutically acceptable salt thereof, as described herein is administered at a dose ranging from about 25 mg to about 50 mg. In one embodiment, bosentan, or a pharmaceutically acceptable salt thereof, as described herein is administered at a dose ranging from about 50 mg to about 150 mg. In one embodiment, bosentan, or a pharmaceutically acceptable salt thereof, as described herein is administered at a dose ranging from about 50 mg to about 100 mg. In one embodiment, bosentan, or a pharmaceutically acceptable salt thereof, as described herein is administered at a dose ranging from about 50 mg to about 75 mg. In one embodiment, bosentan, or a pharmaceutically acceptable salt thereof, as described herein is administered at a dose ranging from about 75 mg to about 150 mg. In one embodiment, bosentan, or a pharmaceutically acceptable salt thereof, as described herein is administered at a dose ranging from about 75 mg to about 100 mg. In one embodiment, bosentan, or a pharmaceutically acceptable salt thereof, as described herein is administered at a dose ranging from about 100 mg to about 1200 mg. In one embodiment, bosentan, or a pharmaceutically acceptable salt thereof, as described herein is administered at a dose ranging from about 10 mg to about 40 mg. In one embodiment, bosentan, or a pharmaceutically acceptable salt thereof, as described herein is administered at a dose ranging from about 10 mg to about 30 mg. In one embodiment, bosentan, or a pharmaceutically acceptable salt thereof, as described herein is administered at a dose ranging from about 10 mg to about 20 mg. In one embodiment, bosentan, or a pharmaceutically acceptable salt thereof, as described herein is administered at a dose ranging from about 15 mg to about 40 mg. In one embodiment, bosentan, or a pharmaceutically acceptable salt thereof, as described herein is administered at a dose ranging from about 20 mg to about 40 mg. In one embodiment, bosentan, or a pharmaceutically acceptable salt thereof, as described herein is administered at a dose ranging from about 30 mg to about 40 mg. In one embodiment, bosentan, or a pharmaceutically acceptable salt thereof, as described herein is administered at a dose of about 10 mg. In one embodiment, bosentan, or a pharmaceutically acceptable salt thereof, as described herein is administered at a dose of about 15 mg. In one embodiment, bosentan, or a pharmaceutically acceptable salt thereof, as described herein is administered at a dose of about 20 mg. In one embodiment, bosentan, or a pharmaceutically acceptable salt thereof, as described herein is administered at a dose of about 25 mg. In one embodiment, bosentan, or a pharmaceutically acceptable salt thereof, as described herein is administered at a dose of about 30 mg. In one embodiment, bosentan, or a pharmaceutically acceptable salt thereof, as described herein is administered at a dose of about 35 mg. In one embodiment, bosentan, or a pharmaceutically acceptable salt thereof, as described herein is administered at a dose of about 40 mg. In one embodiment, bosentan, or a pharmaceutically acceptable salt thereof, as described herein is administered at a dose of about 45 mg. In one embodiment, bosentan, or a pharmaceutically acceptable salt thereof, as described herein is administered at a dose of about 50 mg. In one embodiment, bosentan, or a pharmaceutically acceptable salt thereof, as described herein is administered at a dose of about 55 mg. In one embodiment, bosentan, or a pharmaceutically acceptable salt thereof, as described herein is administered at a dose of about 60 mg. In one embodiment, bosentan, or a pharmaceutically acceptable salt thereof, as described herein is administered at a dose of about 62.5 mg. In one embodiment, bosentan, or a pharmaceutically acceptable salt thereof, as described herein is administered at a dose of about 65 mg. In one embodiment, bosentan, or a pharmaceutically acceptable salt thereof, as described herein is administered at a dose of about 70 mg. In one embodiment, bosentan, or a pharmaceutically acceptable salt thereof, as described herein is administered at a dose of about 75 mg. In one embodiment, bosentan, or a pharmaceutically acceptable salt thereof, as described herein is administered at a dose of about 80 mg. In one embodiment, bosentan, or a pharmaceutically acceptable salt thereof, as described herein is administered at a dose of about 85 mg. In one embodiment, bosentan, or a pharmaceutically acceptable salt thereof, as described herein is administered at a dose of about 90 mg. In one embodiment, bosentan, or a pharmaceutically acceptable salt thereof, as described herein is administered at a dose of about 95 mg. In one embodiment, bosentan, or a pharmaceutically acceptable salt thereof, as described herein is administered at a dose of about 100 mg. In one embodiment, bosentan, or a pharmaceutically acceptable salt thereof, as described herein is administered at a dose of about 105 mg. In one embodiment, bosentan, or a pharmaceutically acceptable salt thereof, as described herein is administered at a dose of about 110 mg. In one embodiment, bosentan, or a pharmaceutically acceptable salt thereof, as described herein is administered at a dose of about 115 mg. In one embodiment, bosentan, or a pharmaceutically acceptable salt thereof, as described herein is administered at a dose of about 120 mg. In one embodiment, bosentan, or a pharmaceutically acceptable salt thereof, as described herein is administered at a dose of about 125 mg. In one embodiment, bosentan, or a pharmaceutically acceptable salt thereof, as described herein is administered at a dose of about 130 mg. In one embodiment, bosentan, or a pharmaceutically acceptable salt thereof, as described herein is administered at a dose of about 135 mg. In one embodiment, bosentan, or a pharmaceutically acceptable salt thereof, as described herein is administered at a dose of about 140 mg. In one embodiment, bosentan, or a pharmaceutically acceptable salt thereof, as described herein is administered at a dose of about 145 mg. In one embodiment, bosentan, or a pharmaceutically acceptable salt thereof, as described herein is administered at a dose of about 150 mg. In one embodiment, bosentan, or a pharmaceutically acceptable salt thereof, as described herein is administered at a dose of about 175 mg. In one embodiment, bosentan, or a pharmaceutically acceptable salt thereof, as described herein is administered at a dose of about 200 mg. In one embodiment, bosentan, or a pharmaceutically acceptable salt thereof, as described herein is administered at a dose of about 250 mg. In one embodiment, bosentan, or a pharmaceutically acceptable salt thereof, as described herein is administered at a dose of about 300 mg. In one embodiment, bosentan, or a pharmaceutically acceptable salt thereof, as described herein is administered at a dose of about 350 mg. In one embodiment, bosentan, or a pharmaceutically acceptable salt thereof, as described herein is administered at a dose of about 400 mg. In one embodiment, bosentan, or a pharmaceutically acceptable salt thereof, as described herein is administered at a dose of about 450 mg. In one embodiment, bosentan, or a pharmaceutically acceptable salt thereof, as described herein is administered at a dose of about 500 mg. In one embodiment, bosentan, or a pharmaceutically acceptable salt thereof, as described herein is administered at a dose of about 550 mg. In one embodiment, bosentan, or a pharmaceutically acceptable salt thereof, as described herein is administered at a dose of about 600 mg. In one embodiment, bosentan, or a pharmaceutically acceptable salt thereof, as described herein is administered at a dose of about 650 mg. In one embodiment, bosentan, or a pharmaceutically acceptable salt thereof, as described herein is administered at a dose of about 700 mg. In one embodiment, bosentan, or a pharmaceutically acceptable salt thereof, as described herein is administered at a dose of about 750 mg. In one embodiment, bosentan, or a pharmaceutically acceptable salt thereof, as described herein is administered at a dose of about 800 mg. In one embodiment, bosentan, or a pharmaceutically acceptable salt thereof, as described herein is administered at a dose of about 850 mg. In one embodiment, bosentan, or a pharmaceutically acceptable salt thereof, as described herein is administered at a dose of about 900 mg. In one embodiment, bosentan, or a pharmaceutically acceptable salt thereof, as described herein is administered at a dose of about 950 mg. In one embodiment, bosentan, or a pharmaceutically acceptable salt thereof, as described herein is administered at a dose of about 1000 mg. In one embodiment, bosentan, or a pharmaceutically acceptable salt thereof, as described herein is administered at a dose of about 1050 mg. In one embodiment, bosentan, or a pharmaceutically acceptable salt thereof, as described herein is administered at a dose of about 1100 mg. In one embodiment, bosentan, or a pharmaceutically acceptable salt thereof, as described herein is administered at a dose of about 1150 mg. In one embodiment, bosentan, or a pharmaceutically acceptable salt thereof, as described herein is administered at a dose of about 1200 mg. In one embodiment, bosentan, or a pharmaceutically acceptable salt thereof, as described herein is administered at a dose of about 1250 mg.

In one embodiment of the methods or uses or product for uses provided herein, an agent that decompresses blood vessels is administered to the subject daily, twice daily, three times daily or four times daily. In one embodiment of the methods or uses or product for uses provided herein, bosentan, or a pharmaceutically acceptable salt thereof, is administered to the subject daily, twice daily, three times daily or four times daily. In some embodiments, bosentan, or a pharmaceutically acceptable salt thereof, is administered to the subject every other day, once about every week or once about every three weeks. In some embodiments, bosentan, or a pharmaceutically acceptable salt thereof, is administered to the subject about once per day. In some embodiments, bosentan, or a pharmaceutically acceptable salt thereof, is administered to the subject about twice per day. In some embodiments, bosentan, or a pharmaceutically acceptable salt thereof, is administered to the subject once per day. In some embodiments, bosentan, or a pharmaceutically acceptable salt thereof, is administered to the subject twice per day. In some embodiments, bosentan, or a pharmaceutically acceptable salt thereof, is administered to the subject orally.

In some embodiments of the methods or uses or product for uses provided herein a chemotherapeutic agent as described herein is administered to the subject at a dose ranging from about 0.5 mg/kg to about 15 mg/kg of the subject's body weight. In some embodiments of the methods or uses or product for uses provided herein a checkpoint inhibitor as described herein is administered to the subject at a dose ranging from about 0.5 mg/kg to about 15 mg/kg of the subject's body weight. In one embodiment, a checkpoint inhibitor as described herein is administered at a dose ranging from about 1 mg/kg to about 10 mg/kg. In one embodiment, a checkpoint inhibitor as described herein is administered at a dose of about 1 mg/kg of the subject's body weight. In one embodiment, a checkpoint inhibitor as described herein is administered at a dose of about 2 mg/kg of the subject's body weight. In one embodiment, a checkpoint inhibitor as described herein is administered at a dose of about 3 mg/kg of the subject's body weight. In one embodiment, a checkpoint inhibitor as described herein is administered at a dose of about 4 mg/kg of the subject's body weight. In one embodiment, a checkpoint inhibitor as described herein is administered at a dose of about 5 mg/kg of the subject's body weight. In one embodiment, a checkpoint inhibitor as described herein is administered at a dose of about 6 mg/kg of the subject's body weight. In one embodiment, a checkpoint inhibitor as described herein is administered at a dose of about 7 mg/kg of the subject's body weight. In one embodiment, a checkpoint inhibitor as described herein is administered at a dose of about 8 mg/kg of the subject's body weight. In one embodiment, a checkpoint inhibitor as described herein is administered at a dose of about 9 mg/kg of the subject's body weight. In one embodiment, a checkpoint inhibitor as described herein is administered at a dose of about 10 mg/kg of the subject's body weight. In one embodiment, a checkpoint inhibitor as described herein is administered at a dose of about 11 mg/kg of the subject's body weight. In one embodiment, a checkpoint inhibitor as described herein is administered at a dose of about 12 mg/kg of the subject's body weight. In one embodiment, a checkpoint inhibitor as described herein is administered at a dose of about 13 mg/kg of the subject's body weight. In one embodiment, a checkpoint inhibitor as described herein is administered at a dose of about 14 mg/kg of the subject's body weight. In one embodiment, a checkpoint inhibitor as described herein is administered at a dose of about 15 mg/kg of the subject's body weight. In one embodiment, a checkpoint inhibitor as described herein is administered at a dose of about 2 mg/kg and the checkpoint inhibitor is pembrolizumab. In one embodiment, a checkpoint inhibitor as described herein is administered at a dose of about 1 mg/kg and the checkpoint inhibitor is nivolumab. In one embodiment, a checkpoint inhibitor as described herein is administered at a dose of about 3 mg/kg and the checkpoint inhibitor is nivolumab. In one embodiment, a checkpoint inhibitor as described herein is administered at a dose of about 1 mg/kg and the checkpoint inhibitor is ipilimumab. In one embodiment, a checkpoint inhibitor as described herein is administered at a dose of about 3 mg/kg and the checkpoint inhibitor is ipilimumab. In one embodiment, a checkpoint inhibitor as described herein is administered at a dose of about 10 mg/kg and the checkpoint inhibitor is ipilimumab.

In some embodiments of the methods or uses or product for uses provided herein a chemotherapeutic agent as described herein is administered to the subject at a dose ranging from about 100 mg to about 2000 mg. In some embodiments of the methods or uses or product for uses provided herein a checkpoint inhibitor as described herein is administered to the subject at a dose ranging from about 100 mg to about 2000 mg. In one embodiment, a checkpoint inhibitor as described herein is administered at a dose ranging from about 200 mg to about 1800 mg. In one embodiment, a checkpoint inhibitor as described herein is administered at a dose ranging from about 200 mg to about 400 mg. In one embodiment, a checkpoint inhibitor as described herein is administered at a dose ranging from about 400 mg to about 600 mg. In one embodiment, a checkpoint inhibitor as described herein is administered at a dose ranging from about 600 mg to about 1000 mg. In one embodiment, a checkpoint inhibitor as described herein is administered at a dose ranging from about 800 mg to about 1000 mg. In one embodiment, a checkpoint inhibitor as described herein is administered at a dose ranging from about 1000 mg to about 1800 mg. In one embodiment, a checkpoint inhibitor as described herein is administered at a dose ranging from about 1000 mg to about 1600 mg. In one embodiment, a checkpoint inhibitor as described herein is administered at a dose ranging from about 1000 mg to about 1300 mg. In one embodiment, a checkpoint inhibitor as described herein is administered at a dose ranging from about 140 mg to about 1800 mg. In one embodiment, a checkpoint inhibitor as described herein is administered at a dose ranging from about 1600 mg to about 1800 mg. In one embodiment, a checkpoint inhibitor as described herein is administered at a dose of about 100 mg. In one embodiment, a checkpoint inhibitor as described herein is administered at a dose of about 200 mg. In one embodiment, a checkpoint inhibitor as described herein is administered at a dose of about 240 mg. In one embodiment, a checkpoint inhibitor as described herein is administered at a dose of about 300 mg. In one embodiment, a checkpoint inhibitor as described herein is administered at a dose of about 360 mg. In one embodiment, a checkpoint inhibitor as described herein is administered at a dose of about 400 mg. In one embodiment, a checkpoint inhibitor as described herein is administered at a dose of about 480 mg. In one embodiment, a checkpoint inhibitor as described herein is administered at a dose of about 500 mg. In one embodiment, a checkpoint inhibitor as described herein is administered at a dose of about 600 mg. In one embodiment, a checkpoint inhibitor as described herein is administered at a dose of about 700 mg. In one embodiment, a checkpoint inhibitor as described herein is administered at a dose of about 800 mg. In one embodiment, a checkpoint inhibitor as described herein is administered at a dose of about 840 mg. In one embodiment, a checkpoint inhibitor as described herein is administered at a dose of about 900 mg. In one embodiment, a checkpoint inhibitor as described herein is administered at a dose of about 1000 mg. In one embodiment, a checkpoint inhibitor as described herein is administered at a dose of about 1100 mg. In one embodiment, a checkpoint inhibitor as described herein is administered at a dose of about 1200 mg. In one embodiment, a checkpoint inhibitor as described herein is administered at a dose of about 1300 mg. In one embodiment, a checkpoint inhibitor as described herein is administered at a dose of about 1400 mg. In one embodiment, a checkpoint inhibitor as described herein is administered at a dose of about 1500 mg. In one embodiment, a checkpoint inhibitor as described herein is administered at a dose of about 1600 mg. In one embodiment, a checkpoint inhibitor as described herein is administered at a dose of about 1700 mg. In one embodiment, a checkpoint inhibitor as described herein is administered at a dose of about 1800 mg. In one embodiment, a checkpoint inhibitor as described herein is administered at a dose of about 1900 mg. In one embodiment, a checkpoint inhibitor as described herein is administered at a dose of about 2000 mg. In one embodiment, a checkpoint inhibitor as described herein is administered at a dose of about 200 mg and the checkpoint inhibitor is pembrolizumab. In one embodiment, a checkpoint inhibitor as described herein is administered at a dose of about 400 mg and the checkpoint inhibitor is pembrolizumab. In one embodiment, a checkpoint inhibitor as described herein is administered at a dose of about 240 mg and the checkpoint inhibitor is nivolumab. In one embodiment, a checkpoint inhibitor as described herein is administered at a dose of about 480 mg and the checkpoint inhibitor is nivolumab. In one embodiment, a checkpoint inhibitor as described herein is administered at a dose of about 360 mg and the checkpoint inhibitor is nivolumab. In one embodiment, a checkpoint inhibitor as described herein is administered at a dose of about 840 mg and the checkpoint inhibitor is atezolizumab. In one embodiment, a checkpoint inhibitor as described herein is administered at a dose of about 1200 mg and the checkpoint inhibitor is atezolizumab. In one embodiment, a checkpoint inhibitor as described herein is administered at a dose of about 1680 mg and the checkpoint inhibitor is atezolizumab.

In one embodiment of the methods or uses or product for uses provided herein, a chemotherapeutic agent as described herein is administered to the subject daily, twice daily, three times daily or four times daily. In one embodiment of the methods or uses or product for uses provided herein, a checkpoint inhibitor as described herein is administered to the subject daily, twice daily, three times daily or four times daily. In some embodiments, a checkpoint inhibitor as described herein is administered once about every week to once about every 8 weeks. In some embodiments, a checkpoint inhibitor described herein is administered once about 1 every week. In some embodiments, a checkpoint inhibitor described herein is administered once about 2 every weeks. In some embodiments, a checkpoint inhibitor described herein is administered once about 3 every weeks. In some embodiments, a checkpoint inhibitor described herein is administered once about 4 every weeks. In some embodiments, a checkpoint inhibitor described herein is administered once about 5 every weeks. In some embodiments, a checkpoint inhibitor described herein is administered once about 6 every weeks. In some embodiments, a checkpoint inhibitor described herein is administered once about 7 every weeks. In some embodiments, a checkpoint inhibitor described herein is administered once about 8 every weeks. In some embodiments, a checkpoint inhibitor described herein is administered once about every 3 weeks and the checkpoint inhibitor is pembrolizumab. In some embodiments, a checkpoint inhibitor described herein is administered at a dose of about 200 mg once about every 3 weeks and the checkpoint inhibitor is pembrolizumab. In some embodiments, a checkpoint inhibitor described herein is administered once about every 6 weeks and the checkpoint inhibitor is pembrolizumab. In some embodiments, a checkpoint inhibitor described herein is administered at a dose of about 400 mg once about every 6 weeks and the checkpoint inhibitor is pembrolizumab. In some embodiments, a checkpoint inhibitor described herein is administered at a dose of about 2 mg/kg of the subject's body weight once about every 3 weeks and the checkpoint inhibitor is pembrolizumab. In some embodiments, a checkpoint inhibitor described herein is administered once about every 2 weeks and the checkpoint inhibitor is nivolumab. In some embodiments, a checkpoint inhibitor described herein is administered at a dose of about 240 mg once about every 2 weeks and the checkpoint inhibitor is nivolumab. In some embodiments, a checkpoint inhibitor described herein is administered once about every 3 weeks and the checkpoint inhibitor is nivolumab. In some embodiments, a checkpoint inhibitor described herein is administered at a dose of about 360 mg once about every 3 weeks and the checkpoint inhibitor is nivolumab. In some embodiments, a checkpoint inhibitor described herein is administered once about every 4 weeks and the checkpoint inhibitor is nivolumab. In some embodiments, a checkpoint inhibitor described herein is administered at a dose of about 480 mg once about every 4 weeks and the checkpoint inhibitor is nivolumab. In some embodiments, a checkpoint inhibitor described herein is administered at a dose of about 1 mg/kg once about every 3 weeks and the checkpoint inhibitor is nivolumab. In some embodiments, a checkpoint inhibitor described herein is administered at a dose of about 3 mg/kg once about every 2 weeks and the checkpoint inhibitor is nivolumab. In some embodiments, a checkpoint inhibitor described herein is administered at a dose of about 3 mg/kg once about every 3 weeks and the checkpoint inhibitor is nivolumab. In some embodiments, a checkpoint inhibitor described herein is administered once about every 3 weeks and the checkpoint inhibitor is ipilimumab. In some embodiments, a checkpoint inhibitor described herein is administered once about every 6 weeks and the checkpoint inhibitor is ipilimumab. In some embodiments, a checkpoint inhibitor described herein is administered at a dose of about 1 mg/kg once about every 3 weeks and the checkpoint inhibitor is ipilimumab. In some embodiments, a checkpoint inhibitor described herein is administered at a dose of about 3 mg/kg once about every 3 weeks and the checkpoint inhibitor is ipilimumab. In some embodiments, a checkpoint inhibitor described herein is administered at a dose of about 10 mg/kg once about every 3 weeks and the checkpoint inhibitor is ipilimumab. In some embodiments, a checkpoint inhibitor described herein is administered at a dose of about 10 mg/kg once about every 12 weeks and the checkpoint inhibitor is ipilimumab. In some embodiments, a checkpoint inhibitor described herein is administered at a dose of about 1 mg/kg once about every 6 weeks and the checkpoint inhibitor is ipilimumab. In some embodiments, a checkpoint inhibitor described herein is administered once about every 2 weeks and the checkpoint inhibitor is atezolizumab. In some embodiments, a checkpoint inhibitor described herein is administered at a dose of about 840 mg once about every 2 weeks and the checkpoint inhibitor is atezolizumab. In some embodiments, a checkpoint inhibitor described herein is administered once about every 3 weeks and the checkpoint inhibitor is atezolizumab. In some embodiments, a checkpoint inhibitor described herein is administered at a dose of about 1200 mg once about every 3 weeks and the checkpoint inhibitor is atezolizumab. In some embodiments, a checkpoint inhibitor described herein is administered once about every 4 weeks and the checkpoint inhibitor is atezolizumab. In some embodiments, a checkpoint inhibitor described herein is administered at a dose of about 1680 mg once about every 4 weeks and the checkpoint inhibitor is atezolizumab. In some embodiments, a checkpoint inhibitor as described herein is administered to the subject by intravenous infusion.

### D. Treatment Outcome

In one aspect, a method of treating cancer with an agent that decompresses blood vessels as described herein and a chemotherapeutic agent as described herein results in an improvement in one or more therapeutic effects in the subject after administration relative to a baseline. In one aspect, a method of treating cancer with bosentan, or a pharmaceutically acceptable salt thereof, as described herein and a checkpoint inhibitor as described herein results in an improvement in one or more therapeutic effects in the subject after administration relative to a baseline. In some embodiments, the one or more therapeutic effects is the size of the tumor derived from the cancer (e.g., solid tumor), the objective response rate, the duration of response, the time to response, progression free survival, overall survival, or any combination thereof. In one embodiment, the one or more therapeutic effects is the size of the tumor derived from the cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with an agent that decompresses blood vessels as described herein and a chemotherapeutic agent as described herein may include the RECIST Criteria 1.1. In one embodiment of the methods or uses or product for uses provided herein, response to treatment with bosentan, or pharmaceutically acceptable salt thereof, as described herein and a checkpoint inhibitor as described herein may include the RECIST Criteria 1.1. The RECIST Criteria 1.1 are as follows:

| | Category | Criteria |
|---|---|---|
| Based on | Complete | Disappearance of all target lesions. Any |
| target lesions | Response (CR) | pathological lymph nodes must have reduction in |
| | | short axis to <10 mm. |
| | Partial Response | ≥30% decrease in the sum of the longest diameter |
| | (PR) | (LD) of target lesions, taking as reference the |
| | | baseline sum of LDs. |
| | Stable Disease | Neither sufficient shrinkage to qualify for PR nor |
| | (SD) | sufficient increase to qualify for PD, taking as |
| | | reference the smallest sum of LDs while in trial. |
| | Progressive | ≥20% (and >5 mm) increase in the sum of the LDs |
| | Disease (PD) | of target lesions, taking as reference the smallest |
| | | sum of the target LDs recorded while in trial or the |
| | | appearance of one or more new lesions. |
| Based on non- | CR | Disappearance of all non-target lesions and |
| target lesions | | normalization of tumor marker level. All lymph |
| | | nodes must be non-pathological in size (<10 mm |
| | | short axis). |
| | SD | Persistence of one or more non-target lesion(s) |
| | | or/and maintenance of tumor marker level above the |
| | | normal limits. |
| | PD | Appearance of one or more new lesions and/or |
| | | unequivocal progression of existing non-target |
| | | lesions. |

In one embodiment of the methods or uses or product for uses provided herein, the effectiveness of treatment with an agent that decompresses blood vessels as described herein and a chemotherapeutic agent as described herein is assessed by measuring the objective response rate. In one embodiment of the methods or uses or product for uses provided herein, the effectiveness of treatment with bosentan, or pharmaceutically acceptable salt thereof, as described herein and a checkpoint inhibitor as described herein is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with an agent that decompresses blood vessels as described herein and a chemotherapeutic agent as described herein is assessed by measuring the size of a tumor derived from the cancer (e.g., solid tumor). In one embodiment of the methods or uses or product for uses provided herein, response to treatment with bosentan, or a pharmaceutically acceptable salt thereof, as described herein and a checkpoint inhibitor as described herein is assessed by measuring the size of a tumor derived from the cancer (e.g., solid tumor). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of bosentan, or a pharmaceutically acceptable salt thereof, as described herein and/or a checkpoint inhibitor as described herein. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of bosentan, or a pharmaceutically acceptable salt thereof, as described herein and/or a checkpoint inhibitor as described herein. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In some embodiments, the size of the tumor derived from the cancer is reduced relative to the size of the tumor before administration of bosentan, or a pharmaceutically acceptable salt thereof, as described herein and a checkpoint inhibitor as described herein. In some embodiments, the size of the tumor derived from the cancer is reduced relative to the size of the tumor before administration of bosentan, or a pharmaceutically acceptable salt thereof, as described herein. In some embodiments, the size of the tumor derived from the cancer is reduced relative to the size of the tumor before administration of a checkpoint inhibitor as described herein.

In one embodiment of the methods or uses or product for uses provided described herein, response to treatment with an agent that decompresses blood vessels and a chemotherapeutic agent as described herein promotes regression of a tumor derived from the cancer (e.g., solid tumor). In one embodiment of the methods or uses or product for uses provided described herein, response to treatment with bosentan, or a pharmaceutically acceptable salt thereof, as described herein and a checkpoint inhibitor as described herein promotes regression of a tumor derived from the cancer (e.g., solid tumor). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of bosentan, or pharmaceutically acceptable salt thereof, as described herein and/or a checkpoint inhibitor as described herein. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of bosentan, or a pharmaceutically acceptable salt thereof, as described herein and/or a checkpoint inhibitor as described herein. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by measuring the size of the tumor by magnetic resonance imaging (MRI). In one embodiment, regression of a tumor is determined by measuring the size of the tumor by computed tomography (CT). In some embodiments, the tumor derived from the cancer regresses relative to the size of the tumor before administration of bosentan, or a pharmaceutically acceptable salt thereof, as described herein and a checkpoint inhibitor as described herein. In some embodiments, the tumor derived from the cancer regresses relative to the size of the tumor before administration of bosentan, or a pharmaceutically acceptable salt thereof, as described herein. In some embodiments, the tumor derived from the cancer regresses relative to the size of the tumor before administration of a checkpoint inhibitor as described herein.

In one embodiment of the methods or uses or product for uses described herein, response to treatment with an agent that decompresses blood vessels as described herein and a chemotherapeutic agent as described herein is assessed by measuring the time of progression free survival after administration of the agent that decompresses blood vessels as described herein and/or the chemotherapeutic agent as described herein. In one embodiment of the methods or uses or product for uses described herein, response to treatment with bosentan, or a pharmaceutically acceptable salt thereof, as described herein and a checkpoint inhibitor as described herein is assessed by measuring the time of progression free survival after administration of bosentan, or a pharmaceutically acceptable salt thereof, as described herein and/or a checkpoint inhibitor as described herein. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of bosentan, or a pharmaceutically acceptable salt thereof, as described herein and/or a checkpoint inhibitor as described herein. In some embodiments, the subject exhibits progression-free survival of at least about 6 months after administration of bosentan, or a pharmaceutically acceptable salt thereof, as described herein and/or a checkpoint inhibitor as described herein. In some embodiments, the subject exhibits progression-free survival of at least about one year after administration of bosentan, or a pharmaceutically acceptable salt thereof, as described herein and/or a checkpoint inhibitor as described herein. In some embodiments, the subject exhibits progression-free survival of at least about two years after administration of bosentan, or a pharmaceutically acceptable salt thereof, as described herein and/or a checkpoint inhibitor as described herein. In some embodiments, the subject exhibits progression-free survival of at least about three years after administration of bosentan, or a pharmaceutically acceptable salt thereof, as described herein and/or a checkpoint inhibitor as described herein. In some embodiments, the subject exhibits progression-free survival of at least about four years after administration of bosentan, or a pharmaceutically acceptable salt thereof, as described herein and/or a checkpoint inhibitor as described herein. In some embodiments, the subject exhibits progression-free survival of at least about five years after administration of bosentan, or a pharmaceutically acceptable salt thereof, as described herein and/or a checkpoint inhibitor as described herein. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of bosentan, or a pharmaceutically acceptable salt thereof, as described herein and/or a checkpoint inhibitor as described herein. In some embodiments, the subject exhibits progression-free survival of at least 6 months after administration of bosentan, or a pharmaceutically acceptable salt thereof, as described herein and/or a checkpoint inhibitor as described herein. In some embodiments, the subject exhibits progression-free survival of at least one year after administration of bosentan, or a pharmaceutically acceptable salt thereof, as described herein and/or a checkpoint inhibitor as described herein. In some embodiments, the subject exhibits progression-free survival of at least two years after administration of bosentan, or a pharmaceutically acceptable salt thereof, as described herein and/or a checkpoint inhibitor as described herein. In some embodiments, the subject exhibits progression-free survival of at least three years after administration of bosentan, or a pharmaceutically acceptable salt thereof, as described herein and/or a checkpoint inhibitor as described herein. In some embodiments, the subject exhibits progression-free survival of at least four years after administration of bosentan, or a pharmaceutically acceptable salt thereof, as described herein and/or a checkpoint inhibitor as described herein. In some embodiments, the subject exhibits progression-free survival of at least five years after administration of bosentan, or a pharmaceutically acceptable salt thereof, as described herein and/or a checkpoint inhibitor as described herein. In some embodiments, response to treatment is assessed by measuring the time of progression free survival after administration of bosentan, or a pharmaceutically acceptable salt thereof, as described herein and a checkpoint inhibitor as described herein. In some embodiments, response to treatment is assessed by measuring the time of progression free survival after administration of bosentan, or a pharmaceutically acceptable salt thereof, as described herein. In some embodiments, response to treatment is assessed by measuring the time of progression free survival after administration of a checkpoint inhibitor as described herein.

In one embodiment of the methods or uses or product for uses described herein, response to treatment with an agent that decompresses blood vessels as described herein and a chemotherapeutic agent as described herein is assessed by measuring the time of overall survival after administration of the agent that decompresses blood vessels as described herein and/or the chemotherapeutic agent as described herein. In one embodiment of the methods or uses or product for uses described herein, response to treatment with bosentan, or a pharmaceutically acceptable salt thereof, as described herein and a checkpoint inhibitor as described herein is assessed by measuring the time of overall survival after administration of bosentan, or a pharmaceutically acceptable salt thereof, as described herein and/or a checkpoint inhibitor as described herein. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of bosentan, or a pharmaceutically acceptable salt thereof, as described herein and/or a checkpoint inhibitor as described herein. In some embodiments, the subject exhibits overall survival of at least about 6 months after administration of bosentan, or a pharmaceutically acceptable salt thereof, as described herein and/or a checkpoint inhibitor as described herein. In some embodiments, the subject exhibits overall survival of at least about one year after administration of bosentan, or a pharmaceutically acceptable salt thereof, as described herein and/or a checkpoint inhibitor as described herein. In some embodiments, the subject exhibits overall survival of at least about two years after administration of bosentan, or a pharmaceutically acceptable salt thereof, as described herein and/or a checkpoint inhibitor as described herein. In some embodiments, the subject exhibits overall survival of at least about three years after administration of bosentan, or a pharmaceutically acceptable salt thereof, as described herein and/or a checkpoint inhibitor as described herein. In some embodiments, the subject exhibits overall survival of at least about four years after administration of bosentan, or a pharmaceutically acceptable salt thereof, as described herein and/or a checkpoint inhibitor as described herein. In some embodiments, the subject exhibits overall survival of at least about five years after administration of bosentan, or a pharmaceutically acceptable salt thereof, as described herein and/or a checkpoint inhibitor as described herein. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least about 12 months, at least eighteen months, at least two years, at least three years, at least four years, or at least five years after administration of bosentan, or a pharmaceutically acceptable salt thereof, as described herein and/or a checkpoint inhibitor as described herein. In some embodiments, the subject exhibits overall survival of at least 6 months after administration of bosentan, or a pharmaceutically acceptable salt thereof, as described herein and/or a checkpoint inhibitor as described herein. In some embodiments, the subject exhibits overall survival of at least one year after administration of bosentan, or a pharmaceutically acceptable salt thereof, as described herein and/or a checkpoint inhibitor as described herein. In some embodiments, the subject exhibits overall survival of at least two years after administration of bosentan, or a pharmaceutically acceptable salt thereof, as described herein and/or a checkpoint inhibitor as described herein. In some embodiments, the subject exhibits overall survival of at least three years after administration of bosentan, or a pharmaceutically acceptable salt thereof, as described herein and/or a checkpoint inhibitor as described herein. In some embodiments, the subject exhibits overall survival of at least four years after administration of bosentan, or a pharmaceutically acceptable salt thereof, as described herein and/or a checkpoint inhibitor as described herein. In some embodiments, the subject exhibits overall survival of at least five years after administration of bosentan, or a pharmaceutically acceptable salt thereof, as described herein and/or a checkpoint inhibitor as described herein. In some embodiments, response to treatment is assessed by measuring the time of overall survival after administration of bosentan, or a pharmaceutically acceptable salt thereof, as described herein and a checkpoint inhibitor as described herein. In some embodiments, response to treatment is assessed by measuring the time of overall survival after administration of bosentan, or a pharmaceutically acceptable salt thereof, as described herein. In some embodiments, response to treatment is assessed by measuring the time of overall survival after administration of a checkpoint inhibitor as described herein.

In one embodiment of the methods or uses or product for uses described herein, response to treatment with an agent that decompresses blood vessels as described herein and a chemotherapeutic agent as described herein is assessed by measuring the duration of response to the agent that decompresses blood vessels as described herein and the chemotherapeutic agent as described herein after administration of the agent that decompresses blood vessels as described herein and/or the chemotherapeutic agent as described herein. In one embodiment of the methods or uses or product for uses described herein, response to treatment with bosentan, or a pharmaceutically acceptable salt thereof, as described herein and a checkpoint inhibitor as described herein is assessed by measuring the duration of response to bosentan, or a pharmaceutically acceptable salt thereof, as described herein and a checkpoint inhibitor as described herein after administration of bosentan, or a pharmaceutically acceptable salt thereof, as described herein and/or a checkpoint inhibitor as described herein. In some embodiments, the duration of response to bosentan, or a pharmaceutically acceptable salt thereof, as described herein and a checkpoint inhibitor as described herein is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of bosentan, or a pharmaceutically acceptable salt thereof, as described herein and/or a checkpoint inhibitor as described herein. In some embodiments, the duration of response to bosentan, or a pharmaceutically acceptable salt thereof, as described herein and a checkpoint inhibitor as described herein is at least about 6 months after administration of bosentan, or a pharmaceutically acceptable salt thereof, as described herein and/or a checkpoint inhibitor as described herein. In some embodiments, the duration of response to bosentan, or a pharmaceutically acceptable salt thereof, as described herein and a checkpoint inhibitor as described herein is at least about one year after administration of bosentan, or a pharmaceutically acceptable salt thereof, as described herein and/or a checkpoint inhibitor as described herein. In some embodiments, the duration of response to bosentan, or a pharmaceutically acceptable salt thereof, as described herein and a checkpoint inhibitor as described herein is at least about two years after administration of bosentan, or a pharmaceutically acceptable salt thereof, as described herein and/or a checkpoint inhibitor as described herein. In some embodiments, the duration of response to bosentan, or a pharmaceutically acceptable salt thereof, as described herein and a checkpoint inhibitor as described herein is at least about three years after administration of bosentan, or a pharmaceutically acceptable salt thereof, as described herein and/or a checkpoint inhibitor as described herein. In some embodiments, the duration of response to bosentan, or a pharmaceutically acceptable salt thereof, as described herein and a checkpoint inhibitor as described herein is at least about four years after administration of bosentan, or a pharmaceutically acceptable salt thereof, as described herein and/or a checkpoint inhibitor as described herein. In some embodiments, the duration of response to bosentan, or a pharmaceutically acceptable salt thereof, as described herein and a checkpoint inhibitor as described herein is at least about five years after administration of bosentan, or a pharmaceutically acceptable salt thereof, as described herein and/or a checkpoint inhibitor as described herein. In some embodiments, the duration of response to bosentan, or a pharmaceutically acceptable salt thereof, as described herein and a checkpoint inhibitor as described herein is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of bosentan, or a pharmaceutically acceptable salt thereof, as described herein and/or a checkpoint inhibitor as described herein. In some embodiments, the duration of response to bosentan, or a pharmaceutically acceptable salt thereof, as described herein and a checkpoint inhibitor as described herein is at least 6 months after administration of bosentan, or a pharmaceutically acceptable salt thereof, as described herein and/or a checkpoint inhibitor as described herein. In some embodiments, the duration of response to bosentan, or a pharmaceutically acceptable salt thereof, as described herein and a checkpoint inhibitor as described herein is at least one year after administration of bosentan, or a pharmaceutically acceptable salt thereof, as described herein and/or a checkpoint inhibitor as described herein. In some embodiments, the duration of response to bosentan, or a pharmaceutically acceptable salt thereof, as described herein and a checkpoint inhibitor as described herein is at least two years after administration of bosentan, or a pharmaceutically acceptable salt thereof, as described herein and/or a checkpoint inhibitor as described herein. In some embodiments, the duration of response to bosentan, or a pharmaceutically acceptable salt thereof, as described herein and a checkpoint inhibitor as described herein is at least three years after administration of bosentan, or a pharmaceutically acceptable salt thereof, as described herein and/or a checkpoint inhibitor as described herein. In some embodiments, the duration of response to bosentan, or a pharmaceutically acceptable salt thereof, as described herein and a checkpoint inhibitor as described herein is at least four years after administration of bosentan, or a pharmaceutically acceptable salt thereof, as described herein and/or a checkpoint inhibitor as described herein. In some embodiments, the duration of response to bosentan, or a pharmaceutically acceptable salt thereof, as described herein and a checkpoint inhibitor as described herein is at least five years after administration of bosentan, or a pharmaceutically acceptable salt thereof, as described herein and/or a checkpoint inhibitor as described herein. In some embodiments, the duration of response is measured after administration of the bosentan, or a pharmaceutically acceptable salt thereof, as described herein and a checkpoint inhibitor as described herein. In some embodiments, the duration of response is measured after administration of bosentan, or a pharmaceutically acceptable salt thereof, as described herein. In some embodiments, the duration of response is measured after administration of a checkpoint inhibitor as described herein.

### V. Compositions

In some aspects, also provided herein are compositions (e.g., pharmaceutical compositions and therapeutic formulations) comprising an agent that decompresses blood vessels as described herein and/or a chemotherapeutic agent as described herein. In some aspects, also provided herein are compositions (e.g., pharmaceutical compositions and therapeutic formulations) comprising bosentan, or a pharmaceutically acceptable salt thereof, as described herein and/or a checkpoint inhibitor as described herein.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000).

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intramolecular interactions. Tonicity agents can be present in any amount between about 0.1% to about 25% by weight or between about 1% to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In some embodiments, a formulation comprising bosentan comprises bosentan hydrate dissolved in ddH₂O containing DMSO, PEG300 and Tween 80. In some embodiments, a formulation comprising bosentan comprises bosentan hydrate dissolved in ddH₂O containing 2% DMSO, 30% PEG300 and 2% Tween 80. In some embodiments, a formulation comprising bosentan comprises bosentan hydrate (S3051, Selleckchem) dissolved in ddH₂O containing 2% DMSO (GK2245, Glentham Life Science), 30% PEG300 (S6704, Selleckchem) and 2% Tween 80 (S6702, Selleckchem).

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

In some embodiments, a composition comprising an agent that decompresses blood vessels as described herein is coadministered with a composition comprising a chemotherapeutic agent as described herein. In some embodiments, a composition comprising bosentan, or a pharmaceutically acceptable salt thereof, as described herein is coadministered with a composition comprising a checkpoint inhibitor as described herein. In some embodiments the coadministration is simultaneous or sequential. In some embodiments, bosentan, or a pharmaceutically acceptable salt thereof, as described herein is administered simultaneously with a checkpoint inhibitor as described herein. In some embodiments, simultaneous means that bosentan, or a pharmaceutically acceptable salt thereof, as described herein and the checkpoint inhibitor as described herein are administered to the subject less than about one hour apart, such as less than about 30 minutes apart, less than about 15 minutes apart, less than about 10 minutes apart or less than about 5 minutes apart. In some embodiments, simultaneous means that bosentan, or a pharmaceutically acceptable salt thereof, as described herein and the checkpoint inhibitor as described herein are administered to the subject less than one hour apart, such as less than 30 minutes apart, less than 15 minutes apart, less than 10 minutes apart or less than 5 minutes apart. In some embodiments, bosentan, or a pharmaceutically acceptable salt thereof, as described herein is administered sequentially with the checkpoint inhibitor as described herein. In some embodiments, sequential administration means that bosentan, or a pharmaceutically acceptable salt thereof, as described herein and the checkpoint inhibitor as described herein are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, bosentan, or a pharmaceutically acceptable salt thereof, as described herein is administered prior to the administration of the checkpoint inhibitor as described herein. In some embodiments, bosentan, or a pharmaceutically acceptable salt thereof, as described herein is administered to the subject beginning at least 1 day prior to the subject being administered the checkpoint inhibitor as described herein. In some embodiments, bosentan, or a pharmaceutically acceptable salt thereof, as described herein is administered to the subject beginning at least 2 days prior to the subject being administered the checkpoint inhibitor as described herein. In some embodiments, bosentan, or a pharmaceutically acceptable salt thereof, as described herein is administered to the subject beginning at least 3 days prior to the subject being administered the checkpoint inhibitor as described herein. In some embodiments, bosentan, or a pharmaceutically acceptable salt thereof, as described herein is administered to the subject beginning at least 4 days prior to the subject being administered the checkpoint inhibitor as described herein. In some embodiments, bosentan, or a pharmaceutically acceptable salt thereof, as described herein is administered to the subject beginning at least 5 days prior to the subject being administered the checkpoint inhibitor as described herein. In some embodiments, bosentan, or a pharmaceutically acceptable salt thereof, as described herein is administered to the subject beginning at least 1 week prior to the subject being administered the checkpoint inhibitor as described herein. In some embodiments, bosentan, or a pharmaceutically acceptable salt thereof, as described herein is administered to the subject beginning at least 2 weeks prior to the subject being administered the checkpoint inhibitor as described herein. In some embodiments, bosentan, or a pharmaceutically acceptable salt thereof, as described herein is administered to the subject beginning at least 3 weeks prior to the subject being administered the checkpoint inhibitor as described herein. In some embodiments, bosentan, or a pharmaceutically acceptable salt thereof, as described herein is administered to the subject beginning at least 4 weeks prior to the subject being administered the checkpoint inhibitor as described herein. In some embodiments, bosentan, or a pharmaceutically acceptable salt thereof, as described herein is administered to the subject beginning prior to the subject being administered the checkpoint inhibitor as described herein and administration is maintained for at least a portion of the time the subject is administered the checkpoint inhibitor. In some embodiments, bosentan, or a pharmaceutically acceptable salt thereof, as described herein is administered to the subject beginning prior to the subject being administered the checkpoint inhibitor as described herein and administration is maintained for the entire period of time the subject is administered the checkpoint inhibitor.

In some embodiments, a composition comprising bosentan, or a pharmaceutically acceptable salt thereof, as described herein and/or a checkpoint inhibitor as described herein is coadministered with one or additional therapeutic agents. In some embodiments the coadministration is simultaneous or sequential.

### VI. Articles of Manufacture and Kits

In another aspect, an article of manufacture or kit is provided which comprises an agent that decompresses blood vessels as described herein and/or a chemotherapeutic agent as described herein. In another aspect, an article of manufacture or kit is provided which comprises bosentan, or a pharmaceutically acceptable salt thereof, as described herein and/or a checkpoint inhibitor as described herein. The article of manufacture or kit may further comprise instructions for use of bosentan, or a pharmaceutically acceptable salt thereof, as described herein and/or a checkpoint inhibitor as described herein in the methods of the invention. Thus, in certain embodiments, the article of manufacture or kit comprises instructions for the use of bosentan, or a pharmaceutically acceptable salt thereof, as described herein and/or a checkpoint inhibitor as described herein in methods for treating cancer (e.g., solid tumors) in a subject comprising administering to the subject an effective amount of bosentan, or a pharmaceutically acceptable salt thereof, as described herein and/or a checkpoint inhibitor as described herein. In some embodiments of any of the aspects provided herein, the solid tumor is selected from the group consisting of breast cancer, breast cancer lung metastases, sarcoma, pancreatic cancer, ovarian cancer, liver metastases, prostate cancer, brain cancer, melanoma, renal cell carcinoma, colorectal cancer, hepatocellular carcinoma, lung cancer, squamous cell carcinoma of the head and neck, urothelial carcinoma, esophageal squamous cell carcinoma, gastric cancer, esophageal cancer, cervical cancer, Merkel cell carcinoma, endometrial carcinoma, mesothelioma, and cutaneous squamous cell carcinoma. In some embodiments, the solid tumor is breast cancer. In some embodiments, the breast cancer has high tumor endothelin-B receptor expression relative to non-tumor tissue. In some embodiments, the breast cancer is triple negative breast cancer. In some embodiments, the solid tumor is a lung metastasis from breast cancer. In some embodiments, the solid tumor is a sarcoma. In some embodiments, the solid tumor is pancreatic cancer. In some embodiments, the solid tumor is ovarian cancer. In some embodiments, the solid tumor is a liver metastasis. In some embodiments, the liver metastasis is from colorectal cancer. In some embodiments, the solid tumor is a prostate cancer. In some embodiments, the prostate cancer has high tumor endothelin-A receptor expression relative to non-tumor tissue. In some embodiments, the solid cancer is a brain cancer. In some embodiments, the brain cancer has high tumor endothelin-A receptor expression relative to non-tumor tissue. In some embodiments, the solid tumor is melanoma. In some embodiments, the solid tumor is renal cell carcinoma. In some embodiments, the solid tumor is colorectal cancer. In some embodiments, the colorectal cancer has high tumor endothelin-A receptor expression relative to non-tumor tissue. In some embodiments, the colorectal cancer has low tumor endothelin-B receptor expression relative to non-tumor tissue. In some embodiments, the colorectal cancer has high tumor endothelin-A receptor expression and low endothelin-B receptor expression relative to non-tumor tissue. In some embodiments, the solid tumor is hepatocellular carcinoma. In some embodiments, the solid tumor is lung cancer. In some embodiments, the lung cancer expresses endothelin-A receptor. In some embodiments, the lung cancer expresses endothelin-B receptor. In some embodiments, the lung cancer expresses both endothelin-A receptor and endothelin-B receptor. In some embodiments, the lung cancer has high tumor endothelin-A receptor expression relative to non-tumor tissue. In some embodiments, the lung cancer has high tumor endothelin-B receptor expression relative to non-tumor tissue. In some embodiments, the lung cancer has high tumor endothelin-A receptor and endothelin-B receptor expression relative to non-tumor tissue. In some embodiments, the lung cancer is non-small cell lung cancer. In some embodiments, the lung cancer is small cell lung cancer. In some embodiments, the solid tumor is squamous cell carcinoma of the head and neck. In some embodiments, the solid tumor is urothelial carcinoma. In some embodiments, the solid tumor is esophageal squamous cell carcinoma. In some embodiments, the solid tumor is gastric cancer. In some embodiments, the solid tumor is esophageal cancer. In some embodiments, the solid tumor is cervical cancer. In some embodiments, the solid tumor is Merkel cell carcinoma. In some embodiments, the solid tumor is endometrial carcinoma. In some embodiments, the solid tumor is mesothelioma. In some embodiments, the solid tumor is cutaneous squamous cell carcinoma. In some embodiments, the solid tumor is a cancer that has compressed blood vessels and/or is hypoperfused. In some embodiments, the solid tumor is a cancer that has compressed blood vessels. In some embodiments, the solid tumor is a cancer that is hypoperfused. In some embodiments, the solid tumor that has compressed blood vessels and/or is hypoperfused is selected from the group consisting of breast cancer, breast cancer lung metastases, pancreatic cancer, ovarian cancer, and liver metastases. In some embodiments, the solid tumor that has compressed blood vessels and/or is hypoperfused is breast cancer. In some embodiments, the breast cancer has high tumor endothelin-B receptor expression relative to non-tumor tissue. In some embodiments, the breast cancer is triple negative breast cancer. In some embodiments, the solid tumor that has compressed blood vessels and/or is hypoperfused is pancreatic cancer. In some embodiments, the solid tumor that has compressed blood vessels and/or is hypoperfused is ovarian cancer. In some embodiments, the solid tumor that has compressed blood vessels and/or is hypoperfused is a liver metastasis. In some embodiments, the liver metastasis is from colorectal cancer. In some embodiments, the solid tumor that has compressed blood vessels and/or is hypoperfused is a lung metastasis. In some embodiments, the liver metastasis is from breast cancer. In some embodiments, the solid tumor is a cancer that has endothelin receptor expression in the tumor vasculature and/or fibroblasts. In some embodiments, the solid tumor is a cancer that has endothelin receptor expression in the tumor vasculature. In some embodiments, the solid tumor is a cancer that has endothelin receptor expression in the tumor fibroblasts. In some embodiments, the solid tumor that has endothelin receptor expression in the tumor vasculature and/or fibroblasts is selected from the group consisting of pancreatic cancer, ovarian cancer, lung cancer, prostate cancer, brain cancer, breast cancer, and colorectal cancer. In some embodiments, the solid tumor that has endothelin receptor expression in the tumor vasculature and/or fibroblasts is pancreatic cancer. In some embodiments, the solid tumor that has endothelin receptor expression in the tumor vasculature and/or fibroblasts is ovarian cancer. In some embodiments, the solid tumor that has endothelin receptor expression in the tumor vasculature and/or fibroblasts is lung cancer. In some embodiments, the lung cancer expresses endothelin-A receptor. In some embodiments, the lung cancer expresses endothelin-B receptor. In some embodiments, the lung cancer expresses both endothelin-A receptor and endothelin-B receptor. In some embodiments, the lung cancer has high tumor endothelin-A receptor expression relative to non-tumor tissue. In some embodiments, the lung cancer has high tumor endothelin-B receptor expression relative to non-tumor tissue. In some embodiments, the lung cancer has high tumor endothelin-A receptor and endothelin-B receptor expression relative to non-tumor tissue. In some embodiments, the lung cancer is non-small cell lung cancer. In some embodiments, the lung cancer is small cell lung cancer. In some embodiments, the solid tumor that has endothelin receptor expression in the tumor vasculature and/or fibroblasts is prostate cancer. In some embodiments, the prostate cancer has high tumor endothelin-A receptor expression relative to non-tumor tissue. In some embodiments, the solid tumor that has endothelin receptor expression in the tumor vasculature and/or fibroblasts is brain cancer. In some embodiments, the brain cancer has high tumor endothelin-A receptor expression relative to non-tumor tissue. In some embodiments, the solid tumor that has endothelin receptor expression in the tumor vasculature and/or fibroblasts is breast cancer. In some embodiments, the breast cancer has high tumor endothelin-B receptor expression relative to non-tumor tissue. In some embodiments, the breast cancer is triple negative breast cancer. In some embodiments, the solid tumor is a lung metastasis from breast cancer. In some embodiments, the solid tumor that has endothelin receptor expression in the tumor vasculature and/or fibroblasts is colorectal cancer. In some embodiments, the colorectal cancer has high tumor endothelin-A receptor expression relative to non-tumor tissue. In some embodiments, the colorectal cancer has low tumor endothelin-B receptor expression relative to non-tumor tissue. In some embodiments, the colorectal cancer has high tumor endothelin-A receptor expression and low endothelin-B receptor expression relative to non-tumor tissue. In some embodiments, the subject is a human.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for intraperitoneal injection, subcutaneous, intravenous (e.g., intravenous infusion), or other modes of administration for treating cancer (e.g., a solid tumor) in a subject. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein bosentan, or a pharmaceutically acceptable salt thereof, as described herein is a first medicament, and which article or kit further comprises instructions on the label or package insert for treating the subject with the second medicament, in an effective amount. In some embodiments, the second medicament is a checkpoint inhibitor as described herein. In some embodiments, the label or package insert indicates that the first and second medicaments are to be administered sequentially or simultaneously, as described herein.

In some embodiments, an agent that decompresses blood vessels as described herein and/or a chemotherapeutic agent as described herein is present in the container as a lyophilized powder. In some embodiments, bosentan, or a pharmaceutically acceptable salt thereof, as described herein and/or a checkpoint inhibitor as described herein is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Examples

### Example 1: Bosentan Normalizes the Mechanical Tumor Microenvironment in a Dose-Dependent Manner

Efficacy of cancer immunotherapy depends on whether T cells can traffic to tumors and migrate to a location adjacent to malignant cells to recognize and kill them. One barrier to T cell homing is the tumor blood vessel wall, which inhibits T cell attachment and transmigration through the endothelin B receptor, but antagonizing this receptor has not yet led to a clinically approved drug. One reason could be hypoperfusion in tumors, which could limit the surface area of perfused blood vessels for anti-tumor T cells to attach. If collapsed tumor blood vessels could be decompressed and reperfused by alleviating mechanical compression (i.e. solid stress), endothelin B receptor antagonism could increase the efficacy of cancer immunotherapy. Furthermore, Endothelin A receptor antagonism inhibits fibrosis in certain disease settings. Bosentan, a non-selective endothelin receptor blocker, was tested herein to determine if it could reduce desmoplasia in cancer.

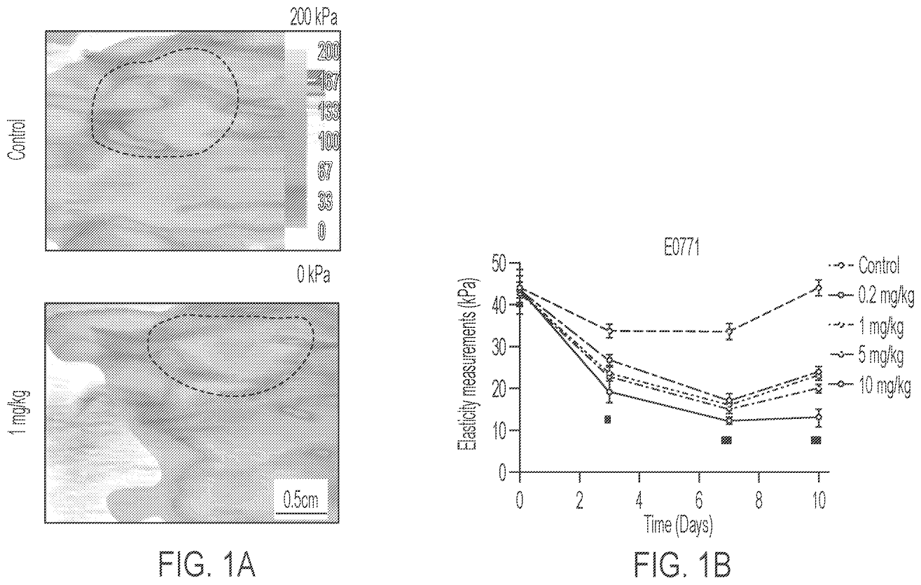

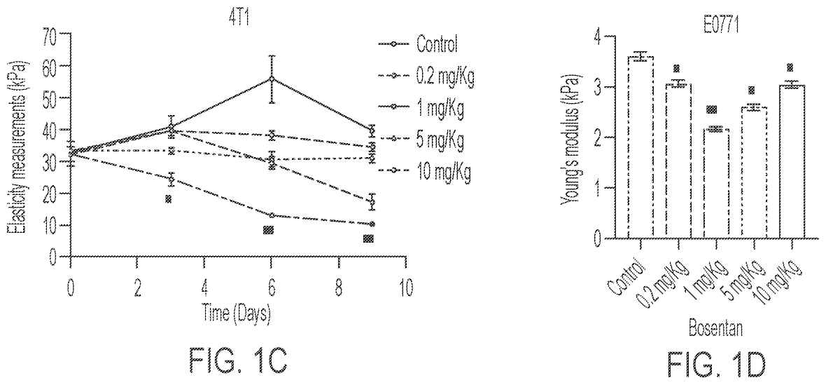

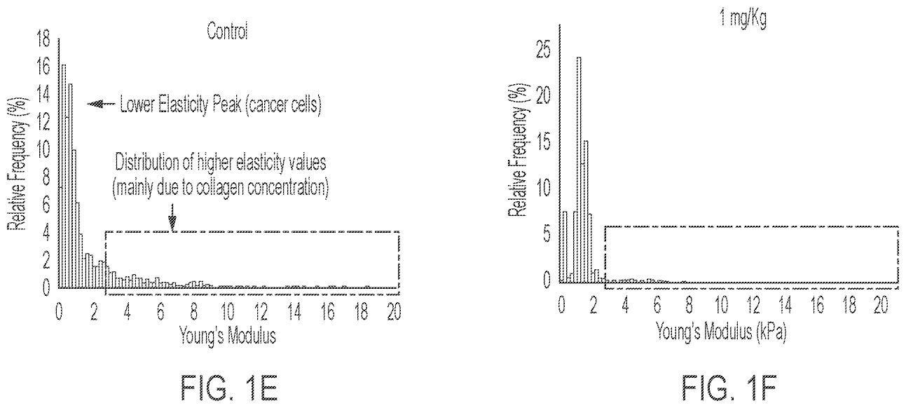

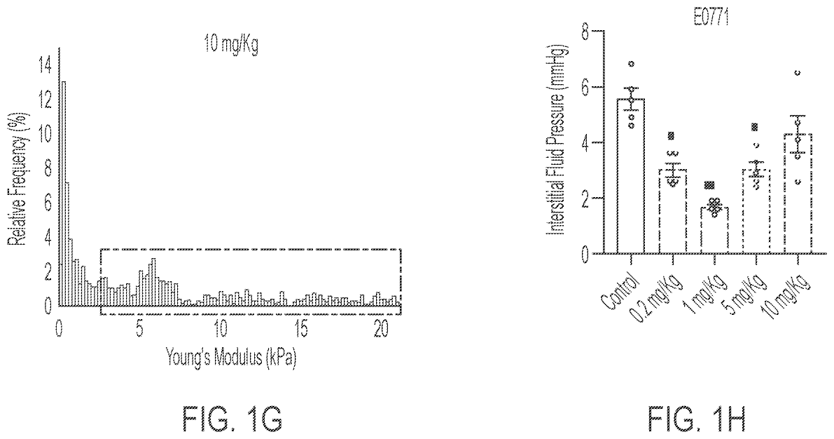

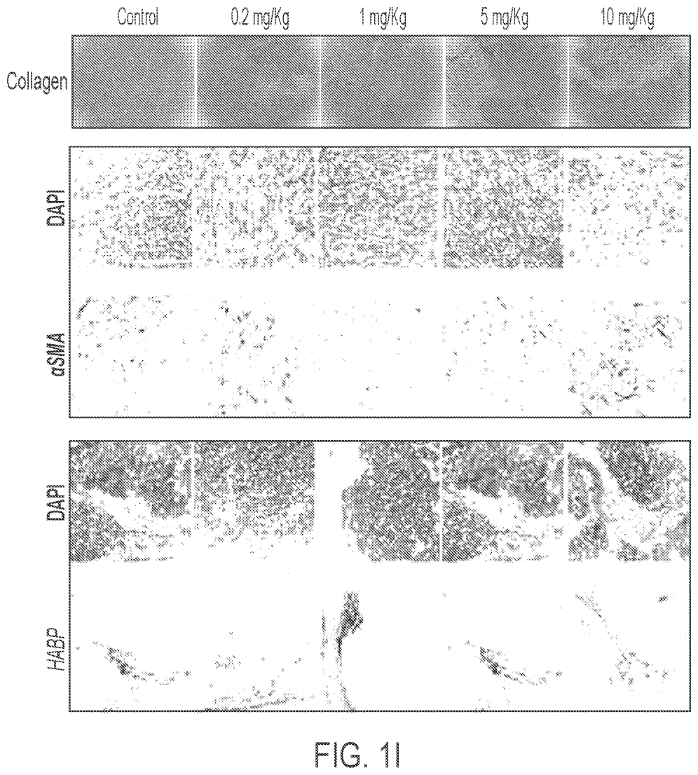

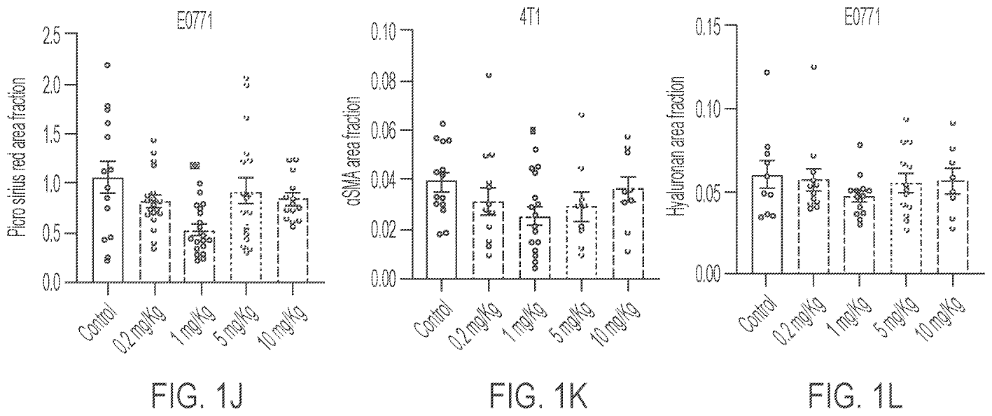

Mice bearing syngeneic, orthotopic triple negative breast cancer (TNBC) were treated with a range of sub-therapeutic doses of bosentan, from 0.2 mg/kg to 10 mg/kg of body weight. Orthotopic models for murine mammary tumors were generated by implantation of 5×10⁴ 4T1 or E0771 cancer cells in 40 μl of serum-free medium into the third mammary fat pad of 6-8-week-old BALB/c and C57BL/6 female mice, respectively. 4T1 (ATCC® CRL-2539TH) and E0771 (94A001, CH3 BioSystems) mouse breast adenocarcinoma cell lines were purchased form ATCC and CH3 BioSystems, respectively. The cells were maintained at 37° C./5% CO₂ in Roswell Park Memorial Institute medium (RPMI-1640, LM-R1637, Biosera) supplemented with 10% fetal bovine serum (FBS, FB-1001H, biosera) and 1% antibiotics (A5955, Sigma). Bosentan (bosentan Hydrate (S3051, Selleckchem) was dissolved in ddH₂O containing 2% DMSO (GK2245, Glentham Life Science), 30% PEG300 (S6704, Selleckchem) and 2% Tween 80 (S6702, Selleckchem)) 1 mg/kg, 5 mg/kg, 10 mg/kg or equal volume of diluent (control group) was administered by intraperitoneal injection (i.p.) once a day for 10 days, starting from once the tumor volumes reached an average size of 100 mm³. Tumors were excised when they reached an average size of 500 mm³. Tissue stiffness was then measured non-invasively and longitudinally using ultrasound elastography. The shear wave imaging method was applied from the system of Philips Epiq Elite Ultrasound, using a handheld linear array (eL18-4) transducer. The method generates shear wave velocity via an acoustic push pulse, creating a color mapped elastogram where red indicates hard and blue soft tissue. A confidence display was also used as a reference of the highest shear wave quality of the user-defined region of interest (ROI). The average elasticity measurements were acquired from the median elasticity values of eight ROIs within the tumor region. The median value of each ROI was automatically generated by the system under default scanner settings and expressed in kPa. For the dose response studies, shear wave imaging of 4T1 tumors was performed prior to bosentan treatment, on day 3, 6 and 9 post-treatment, while imaging of E0771 tumors was performed prior to bosentan administration, on day 3, 7 and 10 post-bosentan treatment. To assess the effect of bosentan 1 mg/kg and ICBs on tissue elasticity, ultrasound was performed prior to any treatment and tumor removal. As shown in FIG. 1A-1C, a moderate dose of 1 mg/kg daily bosentan reduced tissue stiffness in both E0771 and 4T1 breast tumors. As shown in FIG. 1D, this result was confirmed using atomic force microscopy (AFM). For these experiments, during the dose response studies using bosentan, tumors were excised when they reached an average size of 500 mm³. AFM studies were performed using appropriately modified previously published protocols (as in Stylianou A, Lekka M, & Stylianopoulos T (2018) AFM for Assessing Nanomechanical FingerPrints for Cancer Grading and Early Diagnosis: from single cell to tissue level. Nanoscale 10:20930). More specifically, after tumor harvest, tissue biopsies were obtained with an automatic biopsy tool (16G, MEDAX) and the samples were immediately transferred into ice-cold PBS supplemented with a protease inhibitor cocktail (Complete Mini, Roce Dianostics GmbH, 1 tablet per 10 mL) (as in Plodinec M, et al. (2012) The nanomechanical signature of breast cancer. Nature Nanotechnology 7(11):757-765 and Tian M, et al. (2015) The nanomechanical signature of liver cancer tissues and its molecular origin. Nanoscale 7(30):12998-13010). Then each specimen was immobilized on a 35 mm plastic cell culture petri dish with a thin layer of two-component fast drying epoxy glue. The petri dish was filled with PBS supplemented with a protease inhibitor cocktail and stored at 4° C. to avoid tissue degradation. AFM measurements were performed with a commercial AFM system (Molecular Imaging-Agilent PicoPlus AFM) were performed 1-72 h post tumor removal, so as to prevent any alterations in stiffness profiles. The measurements were conducted with silicon nitride cantilevers (MLCT-Bio, cantilever D, Bruker Company with the half-open angle of the pyramidal face of θ˜20°, tip radius: 20 nm, frequency in air: 15 kHz). The maximum applied loading force was set to 1.8 nN, the exact spring constant k of the cantilever was determined before each experiment using the thermal tune method and the deflection sensitivity was determined in fluid using petri dishes as an infinitely stiff reference material (as in Stylianou A, Gkretsi V, Patrickios C S, & Stylianopoulos T (2017) Exploring the Nano-Surface of Collagenous and Other Fibrotic Tissues with AFM. Fibrosis: Methods and Protocols, ed Rittié L (Springer New York, New York, NY), pp 453-489). AFM measurements were performed by recording 10-15 different 20×20 μm² force maps (16×16 point grids) per specimen, which corresponds to 256 force-displacement curves per map (up to 3840 force-displacement curves per specimen) with pixel size of 1.25 μm. Also, for higher spatial resolution, 32×32 force—volume maps (1024 force—displacement curves per map and a pixel size of 0.625 nm) were acquired. The collected force maps were analyzed by AtomicJ (as in Hermanowicz P, Sarna M, Burda K, & Gabryś H (2014) AtomicJ: An open source software for analysis of force curves. Review of Scientific Instruments 85(6):063703) so as to calculate the sample's Young's modulus using the Hertz model (the Poisson ratio, v, was set to 0.5). The mechanical fingerprints of the tissue changed with dose, as control tumors had both contributions from cancer cells and collagen (FIG. 1E), tumors treated with 1 mg/kg had a large contribution from cancer cells and little contribution from collagen (FIG. 1F) and tumors treated with 10 mg/kg had a heterogenous collagen contribution (FIG. 1G). As shown in FIG. 1H, administration of 1 mg/kg bosentan resulted in a decrease in interstitial fluid pressure in E00771 tumors. Given the changes in stiffness, the extracellular matrix molecules hyaluronan binding protein (HABP) and collagen I were directly assessed (see FIG. 1I). The administration of bosentan resulted in a reduction of collagen levels (FIG. 1J), but not aSMA (FIG. 1K) or HABP (FIG. 1L). For these experiments, collagen abundance in E0771 tumor samples was evaluated via Picro Sirius red staining (ab150681, Abcam). Briefly, fixed E0771 samples were dehydrated through a series of graded ethanol washes and embedded in paraffin. Transverse 7 μm-thick paraffin sections were produced using the microtome (Accu-Cut SRM 200 Rotary Microtome, SAKURA), flattened out into water and allowed to dry overnight at 37° C. Sections were then deparaffinized, washed in ddH₂O and incubated in picro Sirius red stain for 1 h at RT. Next, tissue sections were rinsed in two changes of acetic acid, followed by two changes of absolute ethanol and finally, mounted with DPX mountant for histology (Sigma). Collagen fibers are stained in red while the remaining tissue is pale yellow. For hyaluronic acid quantification, E0771 paraffin tumor sections were deparaffinized and rehydrated followed by antigen retrieval (microwave heat treatment with TriSodium Citrate, pH 6, for 20 min). Tissue sections were then washed with 1× TBS/0.025% Triton X-100 (TBS-T), incubated in blocking serum for 2h at RT and then immunostained with the primary biotinylated hyaluronan binding protein (b-HABP) (AMS.HKD-BC41, amsbio 1:100) overnight at 4° C. Hyaluronan was detected following incubation with streptavidin-FITC conjugate (SA1001, Invitrogen1:1000) at RT for 1 h. in the dark. Sections were then mounted on microscope slides using the ProLong gold antifade mountant (Invitrogen) and covered with a glass coverslip. Given the reduced stiffness and collagen I levels, it was hypothesized that lymphatic vessels would be decompressed and hydraulic conductivity reduced leading to reduced interstitial fluid pressure (IFP). Interstitial fluid pressure (IFP) was measured in vivo using the previously described wick-in-needle technique after mice were anesthetized with i.p. injection of Avertin and prior to tumor excision. See Stylianopoulos T, et al. (2012) Causes, consequences, and remedies for growth-induced solid stress in murine and human tumors. Proc. Natl. Acad. Sci. U.S.A 109(38):15101-15108 and Boucher Y, et al. (1990) Interstitial pressure gradients in tissue-isolated and subcutaneous tumors: implications for therapy. Cancer Res 50(15):4478-4484. As shown in FIG. 1H, administration of 1 mg/kg bosentan resulted in a decrease in interstitial fluid pressure in E00771 tumors.

### Example 2: Bosentan Reduces Hypoxia and Increases T Cell Association with Blood Vessels in a Dose Dependent Manner

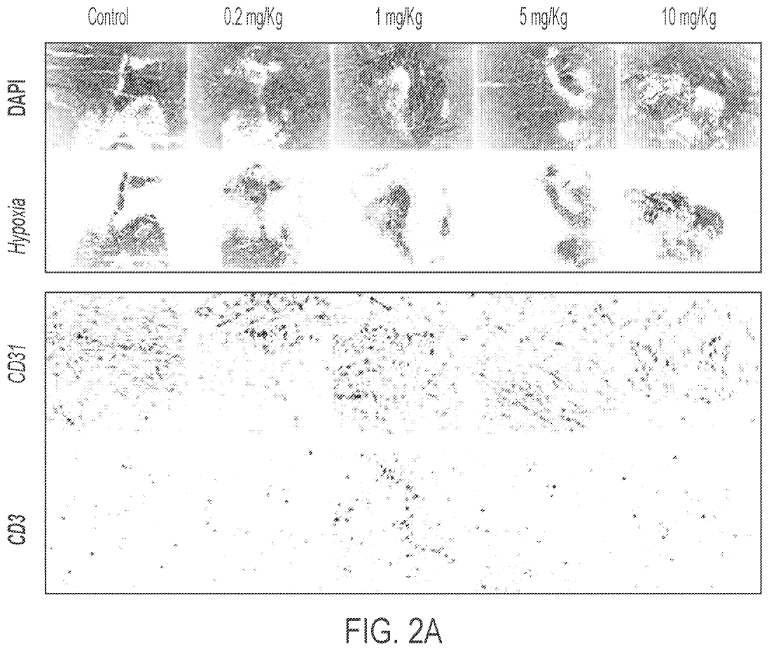

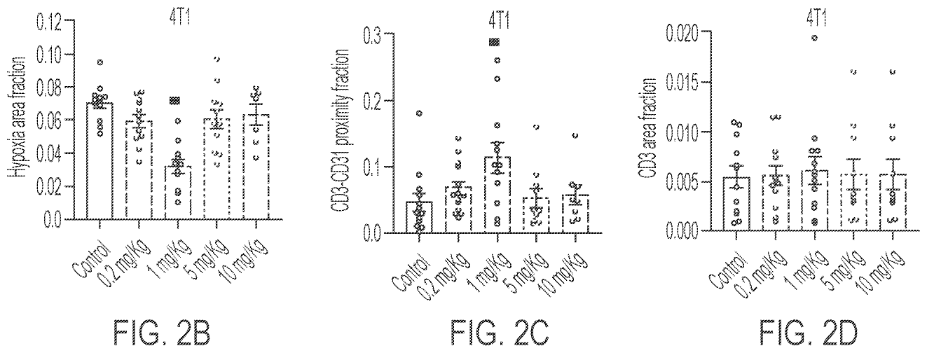

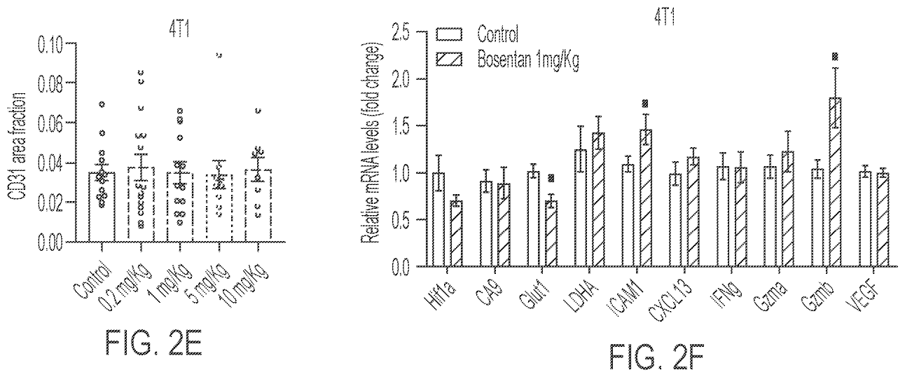

Because reducing collagen I levels and tissue stiffness decompresses vessels, it was hypothesized that hypoxia, which is an indicator of decreased blood flow to the tumor, would be reduced with bosentan treatment. As can be seen in FIGS. 2A and 2B, 1 mg/kg reduced hypoxia in mouse breast tumor models. For the hypoxia studies, Mice bearing orthotopic E0771 or 4T1 breast tumors were injected (intraperitoneal) with 60 mg/kg of pimonidazole HCl at 2 hr prior to tumor removal. Prior to tumors excision, animals were anesthetized via Avertin (200 mg/kg, intraperitoneal). Primary tumors were then excised, fixed in 4% PFA, embedded in OCT and processed accordingly for IHC. Hypoxic regions were detected using the mouse anti-pimonidazole RED 549 conjugate antibody (HP7-100Kit, 1:100). Hypoxic area fraction across different treatment groups was normalized to DAPI staining. For fluorescent immunohistochemistry, tumors were removed, washed twice in 1× PBS for 10 min and incubated with 4% PFA overnight at 4° C. The fixative was aspirated, and samples washed twice in 1× PBS for 10 min. Fixed tissues were embedded in optimal cutting temperature compound in cryomolds (Tissue-Tek) and frozen completely at −20° C. Transverse 30 μm thick tumor sections were produced using the Tissue-Tek Cryo3 (SAKURA). Positively charged HistoBond microscope slides (Marienfeld) were used to bond four tissue sections per tumor. Tumor sections were then incubated in blocking solution (10% fetal bovine serum, 3% donkey serum, 1× PBS) for 2h and immunostained with the following primary antibodies; rabbit anti-Collagen I (ab4710, Abcam 1:100), rabbit anti-CD31 (ab28364, Abcam 1:50), rat anti-CD3 (17A2, BioLegend 1:50) and aSMA (ab5694, Abcam 1:50) overnight at 4° C. Secondary antibodies against rabbit, mouse or rat conjugated to Alexa Fluor 488 or 647 (Invitrogen) were used at 1:400 dilution. All samples were incubated in secondary antibody solution including DAPI (Sigma, 1:100 of 1 mg/mL stock) for 2h at room temperature (RT) in the dark. Sections were mounted on microscope slides using the ProLong gold antifade mountant (Invitrogen) and covered with a glass coverslip. The increased blood flow, as determined by reduced hypoxia, resulted in more T cells colocalizing with endothelial cells (FIG. 2C), despite no change in the area fractions of CD3+ and CD31+stainings (FIGS. 2D and 2E). To demonstrate the presence of T cells in the proximity of tumor vessel capillaries, tissue cryosections of 4T1 and E0771 primary tumors were incubated with primary rabbit anti-CD31 (ab28364, Abcam 1:50) and rat anti-CD3 (17A2, BioLegend 1:50) overnight at 4° C. CD31 signal was detected with Alexa Fluor-488 anti-rabbit IgG (H+L) and CD3 signal with Alexa Fluor-647 anti-rat IgG (H+L) secondary antibodies. Tumor associated T cell and vessel content was determined by the CD31 and CD3 area fraction normalized to DAPI staining. Whole tumor RT-PCR mRNA expression levels supported these findings, as hypoxia related gene-expression was reduced, endothelial adhesion molecule expression as increased, and T cell activity expression was increased (FIG. 2F). For this experiment, Total RNA was isolated from breast tumors according to the standard Trizol-based protocol (Invitrogen), and cDNA synthesis was performed using reverse transcriptase III (RT-III) enzyme and random hexamers (Invitrogen). Real-time polymerase chain reaction was performed using Sybr Fast Universal Master Mix (KAPA). The specific mouse primers used for gene expression analysis of 4T1 tumors are listed in the table below. Reactions were performed using a CFX-96 real-time PCR detection system (Biorad) at the following conditions: 95° C. for 2 min, 95° C. for 2 s, 60° C. for 20 s, 60° C. for 1 s, steps 2-4 for 39 cycles. Real-time PCR analysis and calculation of changes in gene expression between compared groups was performed using the ΔΔ^{Ct} method. Relative gene expression was normalized based on the expression of β-actin and GAPDH. Primer sequences are shown in the following table:

Primer sequences

| Name | Forward sequence | Reverse sequence |
|---|---|---|
| ICAM-1 | CCTGTTTCCTGCCTCTGAAG | TTAAGGTCCTCTGCGTCTCC |
| | (SEQ ID NO: 1) | (SEQ ID NO: 2) |
| Gzma | TGCTGCCCACTGTAACGTG | GGTAGGTGAAGGATAGCCAC |
| | (SEQ ID NO: 3) | AT |
| | | (SEQ ID NO: 4) |
| Gzmb | CCACTCTCGACCCTACATGG | GGCCCCCAAAGTGACATTTA |
| | (SEQ ID NO: 5) | TT |
| | | (SEQ ID NO: 6) |
| IFNg | ATGAACGCTACACACTGCATC | CCATCCTTTTGCCAGTTCCTC |
| | (SEQ ID NO: 7) | (SEQ ID NO: 8) |
| Glut1 | GCTGTGCTTATGGGCTTCTC | ACACCTGGGCAATAAGGATG |
| | (SEQ ID NO: 9) | (SEQ ID NO: 10) |
| FasL | TCCGTGAGTTCACCAACCAAA | GGGGGTTCCCTGTTAAATGGG |
| | (SEQ ID NO: 11) | (SEQ ID NO: 12) |
| LDHA | GGGCTGACAAGATTCATGGT | TTTGGCCACACACTCCAATA |
| | (SEQ ID NO: 13) | (SEQ ID NO: 14) |
| Hif1α | CAGTCGACACAGCCTCGATA | CGGCTCATAACCCATCAACT |
| | (SEQ ID NO: 15) | (SEQ ID NO: 16) |
| CA9 | GCCAGTCCATGTGAATTCCT | CAGCAAAGAGAAGGCCAAAC |
| | (SEQ ID NO: 17) | (SEQ ID NO: 18) |
| CXCL13 | TCGTGCCAAATGGTTACAAA | GCTTGGGGAGTTGAAGACAG |
| | (SEQ ID NO: 19) | (SEQ ID NO: 20) |
| VEGF | AGCACAGCAGATGTGAATGC | TTTCTTGCGCTTTCGTTTTT |
| | (SEQ ID NO: 21) | (SEQ ID NO: 22) |
| β- | GACGGCCAGGTCATCACTAT | AAGGAAGGCTGGAAAAGAGC |
| actin | (SEQ ID NO: 23) | (SEQ ID NO: 24) |
| GAPDH | AACCCTTAAGAGGGATGCTGC | ACGGGACGAAACACTCT |
| | (SEQ ID NO: 25) | (SEQ ID NO: 26) |

Bosentan monotherapy at any dose did not affect tumor growth and mouse body weight.

### Example 3: Bosentan Potentiates Immune Checkpoint Blockade (ICB) Efficacy in Triple Negative Breast Cancer (TNBC)

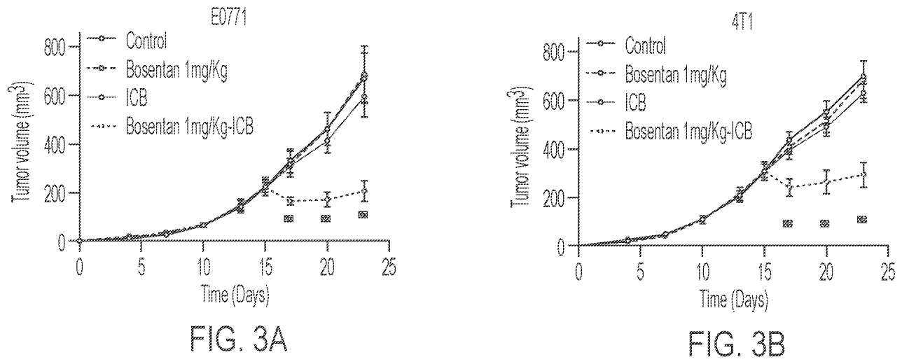

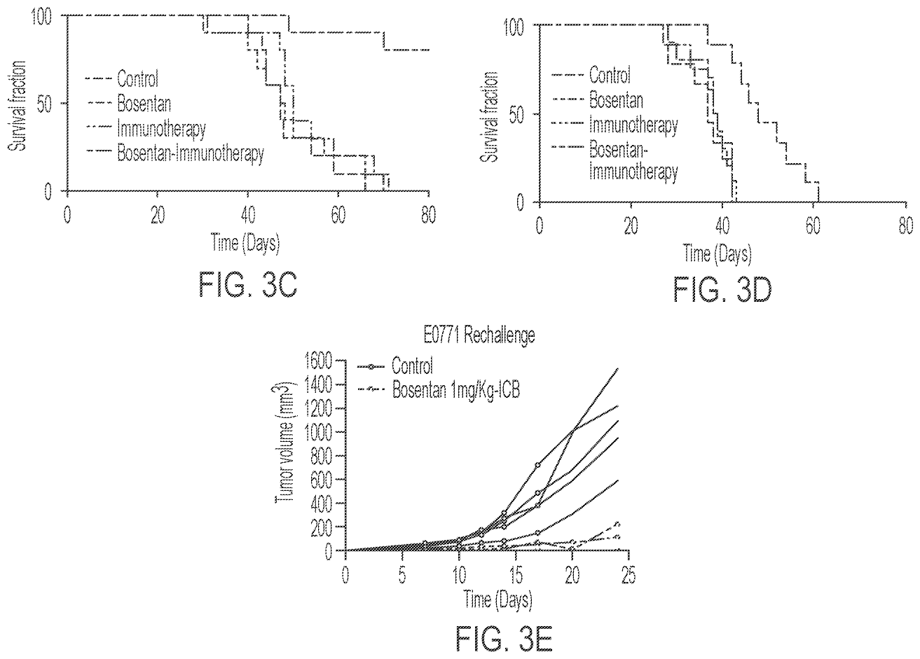

To determine if a stiffness-reducing and hypoxia-reducing (blood flow-increasing) regimen of bosentan could enhance the efficacy of an ICB cocktail of anti-PD-1 (mouse monoclonal anti-PD-1 CD279, clone RMP1-14, BioXCell) and anti-CTLA-4 (mouse monoclonal anti-CTLA-4 CD152, clone 9D9, BioXCell) antibodies, mice bearing primary tumors in a neo-adjuvant setting were administered various treatments. Therapy was administered before removing the primary tumors surgically to assess mice survival against spontaneous metastases that arose on treatment. For the combinatorial treatment with bosentan and Immunotherapy studies, bosentan 1 mg/kg or equal volume of diluent (control group) was administered by intraperitoneal injection (i.p.) once a day for 14 days, starting from once the tumor volumes reached an average size of 5 mm diameter. Immunotherapy was administrated as a cocktail of 10 mg/kg anti-PD-1 (CD279, clone RMP1-14, BioXCell) and 5 mg/kg anti-CTLA-4 (CD152, clone 9D9) following dilution in the recommended InVivoPure pH 7.0 Dilution Buffer (BioXCell). The immunotherapy cocktail was administered i.p. when tumors reached an average size of 200 mm³ every three days for three doses. For the immunotherapy studies, animals were also treated with a non-targeting isotype control antibody (BioXCell). Primary tumors were excised when they reached an average size of 700 mm³ and the tissue was sutured for the study of the metastatic tumors. Planar dimensions (x, y) of tumor were monitored every 2-3 days using a digital caliper and tumor volume was estimated from the volume of an ellipsoid and assuming that the third dimension, z, is equal to x y. For the overall survival studies, the ending point was the time to mouse death. E0771 and 4T1 are resistant to ICB, but bosentan potentiated tumor growth inhibition (FIGS. 3A and 3B) and survival (FIGS. 3C and 3D). In the E0771 study, 8 out of 10 mice initiated survived. Three of the mice were sacrificed and no evidence of macrometasteses were found in the lungs. The remaining five mice were rechallenged with a second inoculation of E0771 cells and the tumor growth rates were compared against healthy age-matched control mice. The tumors grew much slower in the rechallenged mice, with tumors appearing in only two out of the five mice (FIG. 3E).

### Example 4: Ultrasound Biomarkers Correlate to Response

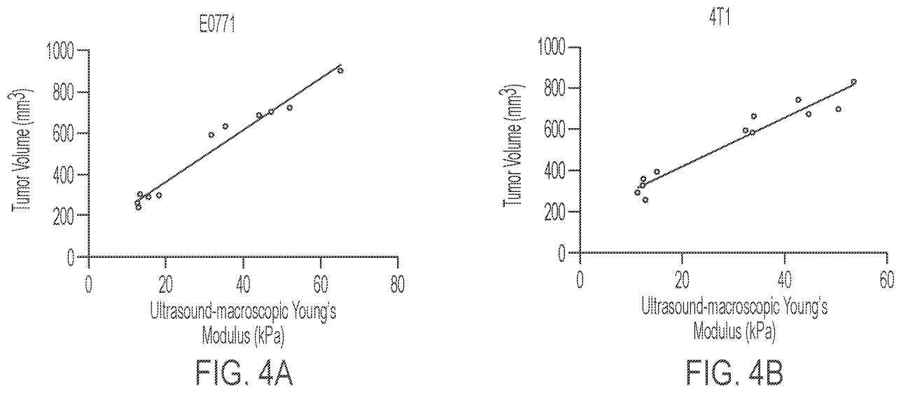

It was next investigated whether the ultrasound elasticity measurements correlate with the tumor response to ICB. It was determined that the Young's modulus of the tumors measured before the initiation of ICB treatment positively correlated well with reduced tumor size across ICB cocktail monotherapy and combination of bosentan and ICB cocktail treatment groups in E0771 (FIG. 4A) and 4T1 tumors (FIG. 4B). The results suggest that tumor response to therapy is noticeably increased when stiffness values drop below 20 kPa for the tumor models considered.

### Example 5: Effect of Bosentan and Immune Checkpoint Blockade in a Mouse Tumor Model

Cell culture, drugs, and reagents. 4T1 murine breast adenocarcinoma cell lines were maintained at 37° C./5% CO2 in RPMI-1640 (cat. number LM-R1637; Biosera) supplemented with 10% fetal bovine serum (cat. number FB-1001H; Biosera) and 1% antibodies (cat. number A5955; Sigma). Bosentan hydrate (cat. number S3051; Selleckchem) was dissolved in ddH2O containing 2% DMSO, 30% PEG300, and 2% Tween 80. The immune checkpoint inhibitor mouse monoclonal anti-PD-1 (CD279; clone RMP1-14) and mouse monoclonal anti-CTLA-4 (CD152; clone 9D9) antibodies were purchased from BioXCell.

Syngeneic tumor models and treatment protocols. Orthotopic models for murine mammary tumors were generated by implantation of 5×10⁴ 4T1 cancer cells in 40 μL of serum-free medium into the third mammary fat pad of 6-8 weeks-old BALB/c mice. Mice were purchased from the Cyprus Institute of Neurology and Genetics and all in vivo experiments were conducted in accordance with the animal welfare regulations and guidelines of the Republic of Cyprus and the European Union (European Directive 2010/63/EE and Cyprus Legislation for the protection and welfare of animals, Laws 1994-2013) under a license acquired and approved (No CY/EXP/PR.L2/2018, CY/EXP/PR.L14/2019, CY/EXP/PR.L15/2019, CY/EXP/PR.L03/2020) by the Cyprus Veterinary Services committee, the Cyprus national authority for monitoring animal research for all academic institutions.

Tranilast at 200 mg/kg, bosentan at 1 mg/kg, or equal volume of diluent (control group) was administered by intraperitoneal injection (i.p.) once a day for 14 days, starting from once the tumor volumes reached an average size of 5 mm diameter. Immunotherapy was administered as a cocktail of 10 mg/kg anti-PD-1 and 5 mg/kg anti-CTLA-4 following dilution in InVivoPure pH 7.0 dilution buffer (BioXCell). The immunotherapy cocktail was administered i.p. when 4T1 tumors reached an average size of 300 mm 3 on days 19, 22, and 25. For the immunotherapy studies, animals were also treated with a non-targeting isotype control antibody (BioXCell).

Planar dimensions (x, y) of tumor were monitored every 2-3 days using a digital caliper and tumor volume was estimated from the volume of an ellipsoid and assuming that the third dimension, z, is equal to the sqrt(x y). For the overall survival studies, the ending point was the time to mouse death.

Ultrasound elastography. Shear wave elestography was employed on a premier Philips EPIQ Elite Ultrasound system using a handheld linear array (eL 18-4) transducer. The method generates shear wave velocity via an acoustic push pulse, creating a color mapped elastogram where red indicates hard tissue and blue indicates soft tissue. A confidence display was also used as a reference of the highest shear wave quality of the user-defined region of interest (ROI). The average value of the tumor region was automatically generated by the system under default scanner settings and expressed in kPa. The settings that were used were: frequency, 10 MHz; power, 52%; B-mode gain, 22 dB; dynamic range, 62 dB. Shear wave imaging was performed prior to immunotherapy cocktail treatment on day 19 and as well on days 23 and 26.

Dynamic contrast-enhanced ultrasound (DCEUS). Tumor associated vascular perfusion was assessed with DCEUS after bolus injection of contrast agents (8 μl of sulphur hexafluoride microbubbles encapsulated by a phospholipid shell with a mean diameter of 2.5 μm, retro-orbital administration). Ultrasound scanning of tumor was performed using the L12-5 transducer. Contrast first harmonic signals were received at 8 MHz with a mechanical index of 0.06. For all subjects the depth was set to 3 cm allowing measurements of the full depth of the tumor. Gain was set at 90% for each recording. Focus was optimized and standardized for each subject when finding the tumor area using B-mode imaging. Real-time power modulation imaging was started after flashing imaging with a high mechanical index to destroy the microbubbles in tumor tissue to peak contrast intensity to allow visualization of bubble replenishment. Image analysis was performed offline using an ultrasound quantification and analysis software (QLAB, Phillips Medical Systems). From the produced time intensity curves, we used as measures of perfusion the Mean transit time and the time required to reach the peak intensity (Rise time). Prior to each ultrasound application, mice were anesthetized by i.p. injection of Avertin (200 mg/kg) and ultrasound gel was applied to the imaging region to prevent any pressure of the transducer on the underlying tissue.

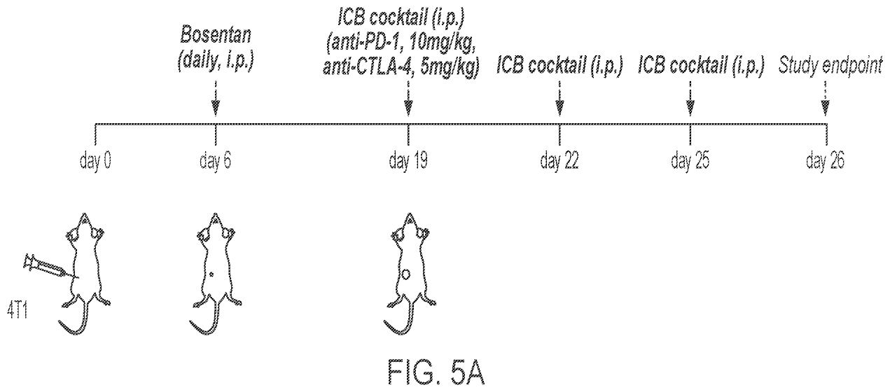

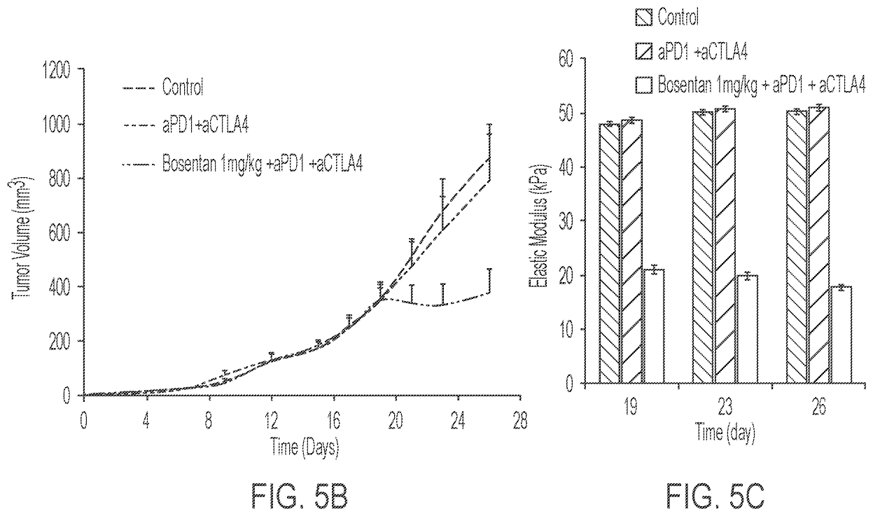

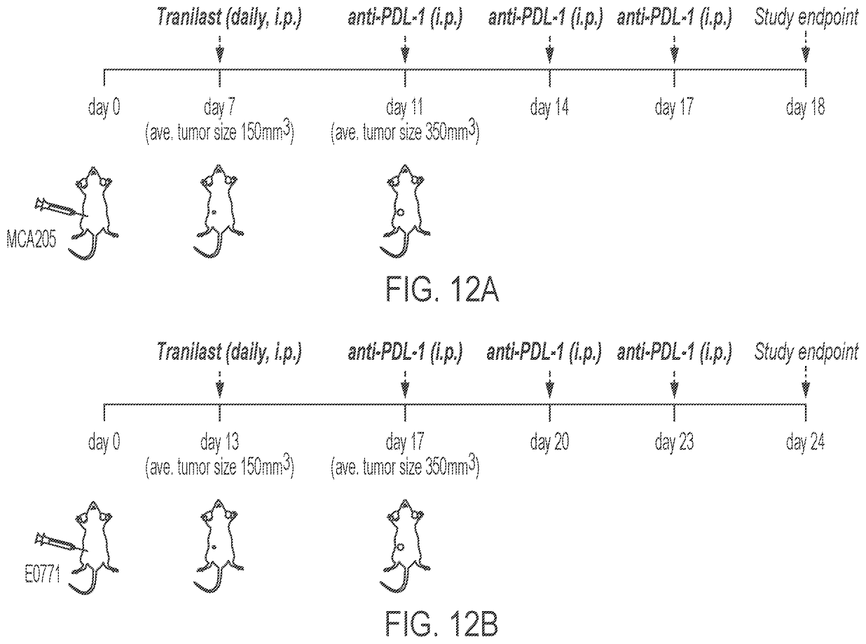

Results. The experimental protocols detailed above were employed for the treatment of mice bearing 4T1 tumors with bosentan and immune checkpoint blockade (ICB) cocktail as shown in FIG. 5A. The experimental protocols were also employed for the treatment of mice bearing MCA205 (experimental protocol shown in FIG. 12A) or E0771 (experimental protocol shown in FIG. 12B) tumors with tranilast. Bosentan treatment was initiated 6 days after cell implantation and tranilast treatment was initiated 7 days after cell implantation. Anti-PD-1+CTLA-4 therapy was initiated when tumors reached an average size of 300 mm³ for bosentan treated mice (FIG. 5A). For mice bearing MCA205 tumors treated with tranilast, tranilast treatment was initiated at day 7 when average tumor volume was about 150 mm³ and anti-PD-L1 therapy was initiated at day 11 when average tumor volume was about 300 mm³. For mice bearing E0771 tumors treated with tranilast, tranilast treatment was initiated at day 13 when average tumor volume was about 150 mm³ and anti-PD-L1 therapy was initiated at day 17 when average tumor volume was about 350 mm³.

The tumor volume as a function of time indicates that when tumors are treated with the immunotherapy cocktail only, they do not respond to the treatment and the growth rate is the same as that of the control group (FIG. 5B). However, when tumors are pre-treated with bosentan, immunotherapy can effectively stop further tumor growth (FIG. 5B). Bosentan treatment reduced the stiffness of the tumors by more than half to values close to 20 kPa, which did not change significantly during immunotherapy-alone administration (FIG. 5C), indicating a connection between bosentan treatment, tumor softening, and efficacy of immunotherapy.

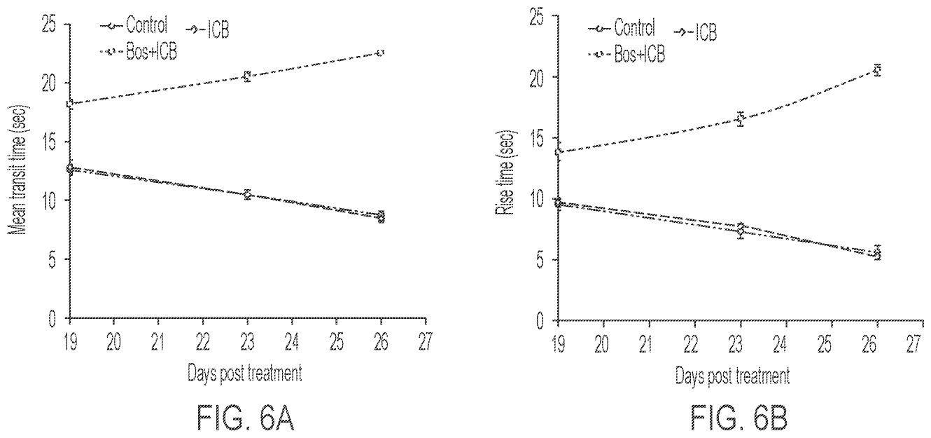

From the quantification of the DCEUS time intensity curves, two measures of perfusion were calculated: the mean transit time and the rise time (FIG. 6A-6B). Both measures were higher in the bosentan treated tumors compared to the control tumors or the tumors that received only the immunotherapy cocktail. The results indicate a relationship between the softening of the tumors as seen in FIG. 5C and the increase in tumor perfusion.

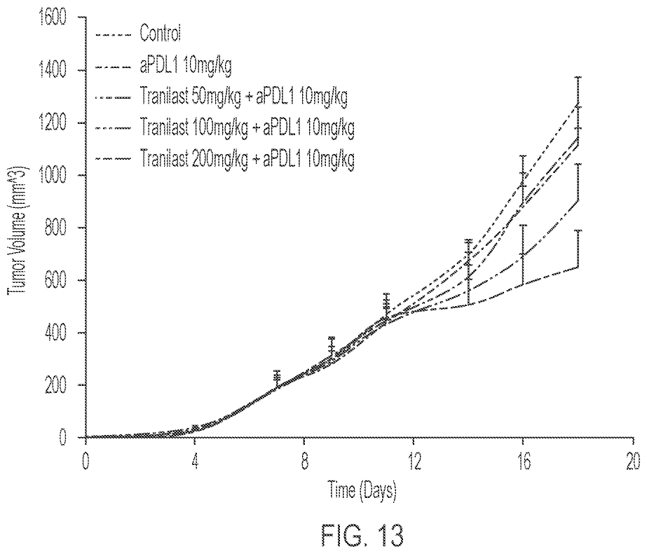

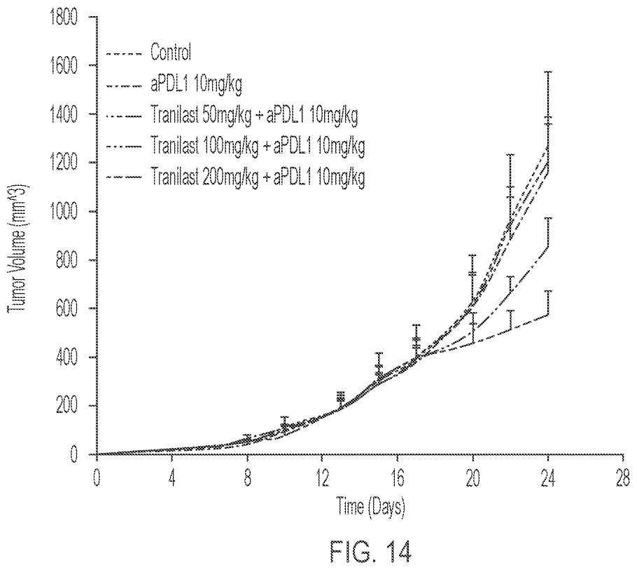

As shown in FIG. 13 for mice bearing MCA205 tumors, anti-PD-L1 therapy alone was only mildly effective at reducing tumor volume compared to control. However, with tranilast pretreatment of 100 mg/kg or 200 mg/kg, the anti-PD-L1 therapy was significantly more effective at reducing tumor volume. Similar results were demonstrated for mice bearing E0771 tumors (FIG. 14).

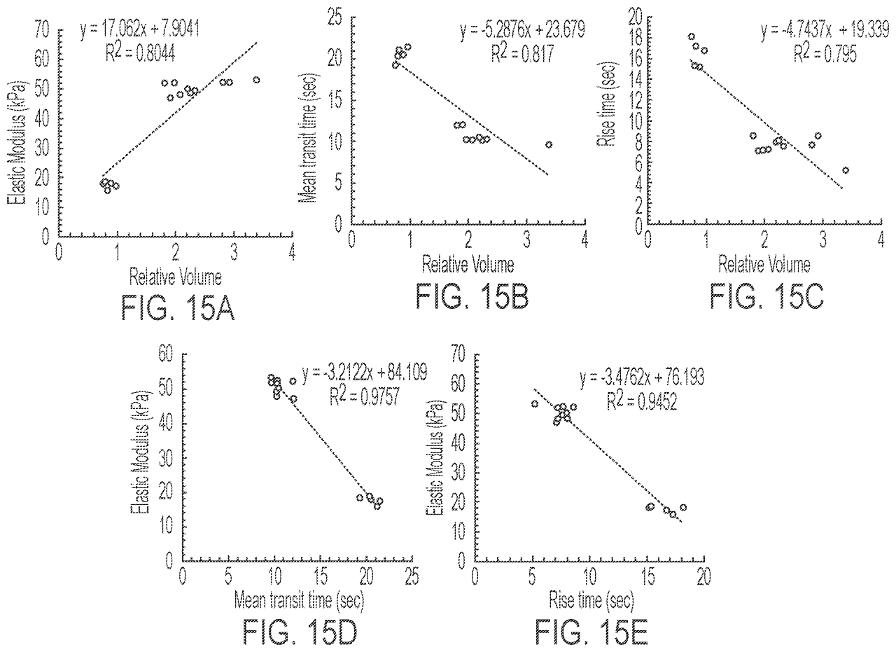

To further investigate the correlation between stiffness, perfusion, and therapeutic efficacy of immunotherapy, the relative change in tumor volume, from the time checkpoint treatment was initiated and the last day of the experiment, a series of correlations was plotted (FIG. 15A-15E) for both tranilast and bosentan-treated mice. For both therapies, a strong and of equal quality correlation was shown between tumor stiffness and the two measures of perfusion with the relative change in the tumor volume. Also, a strong correlation was shown between the elastic modulus and the tumor perfusion.

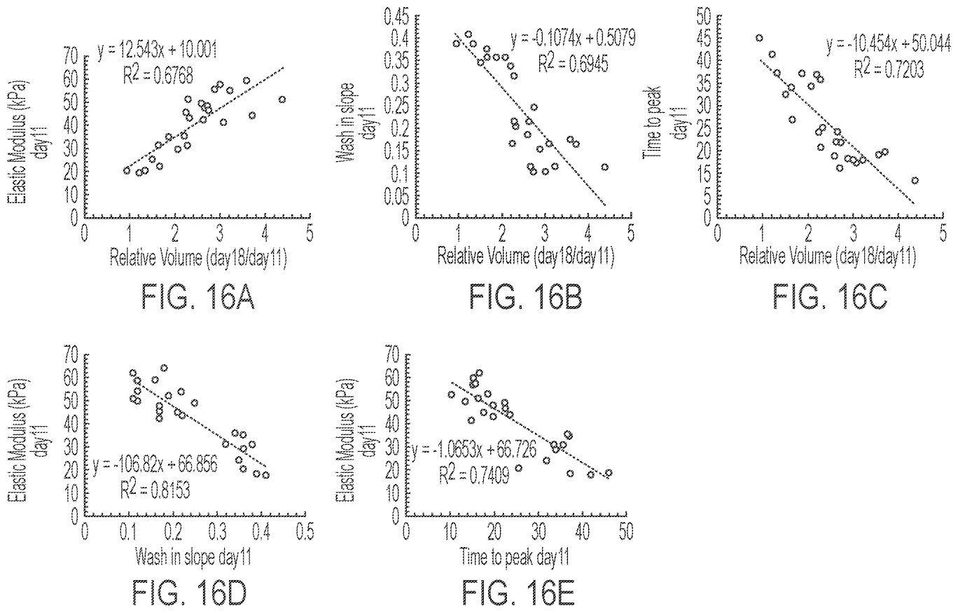

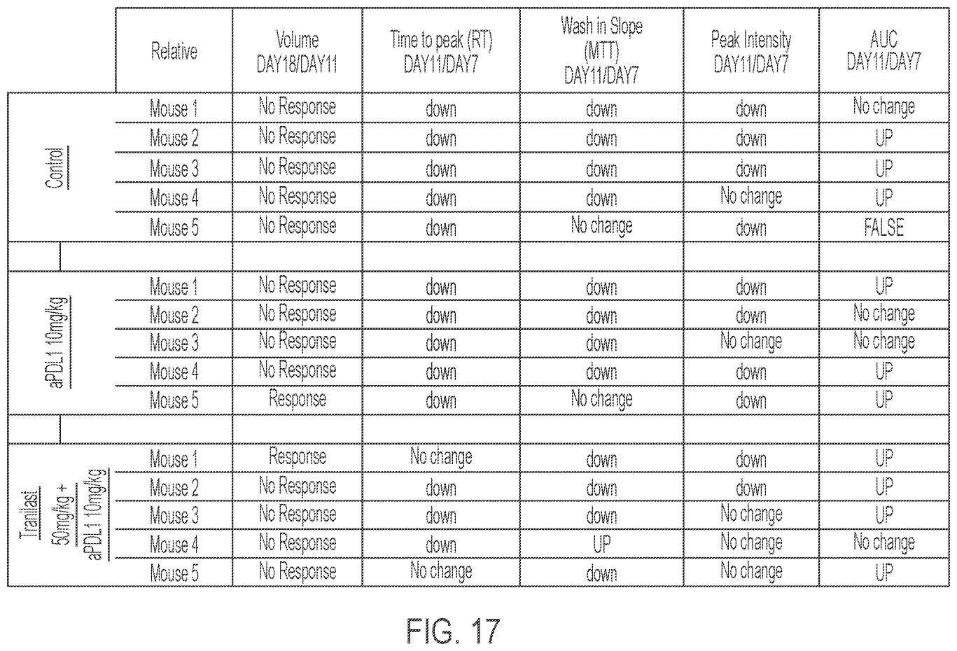

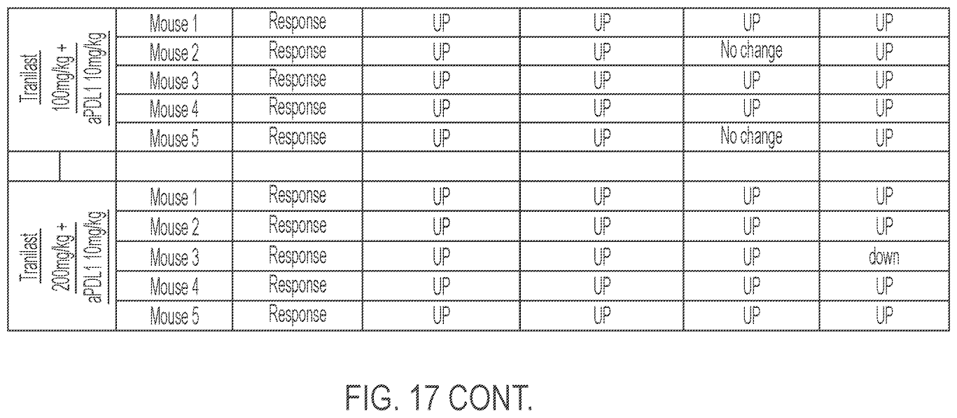

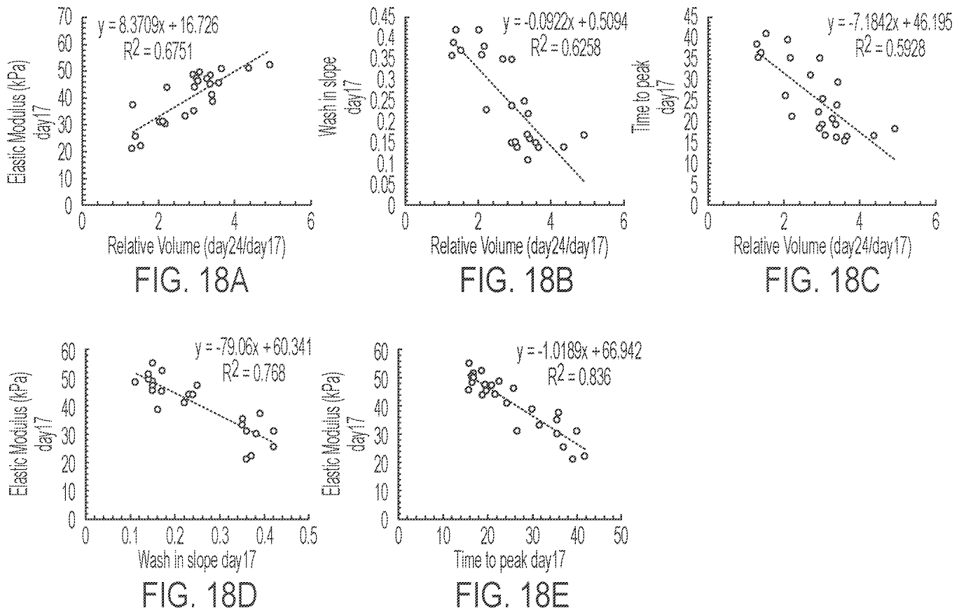

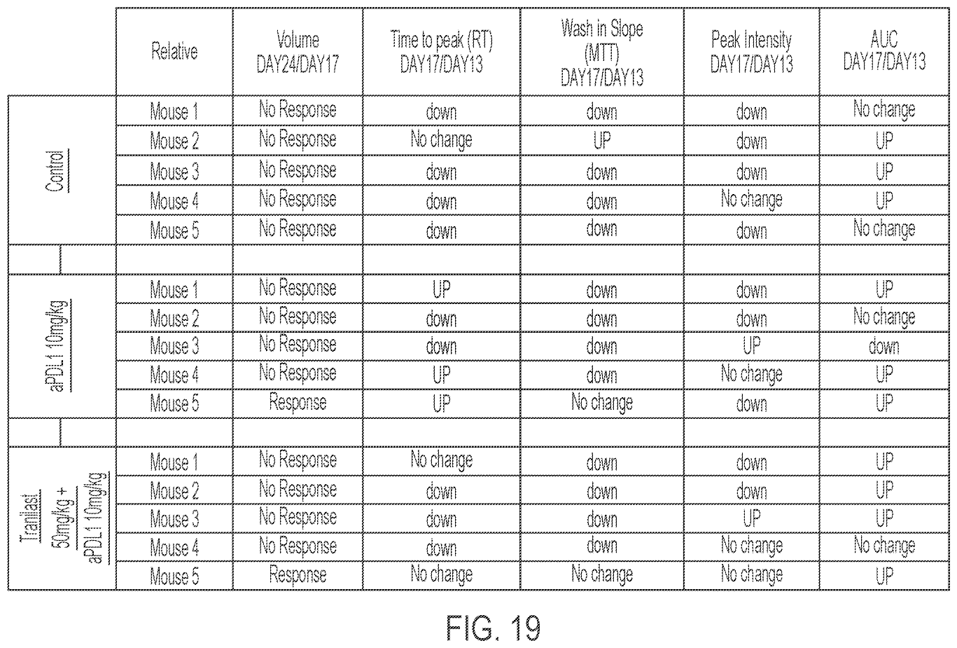

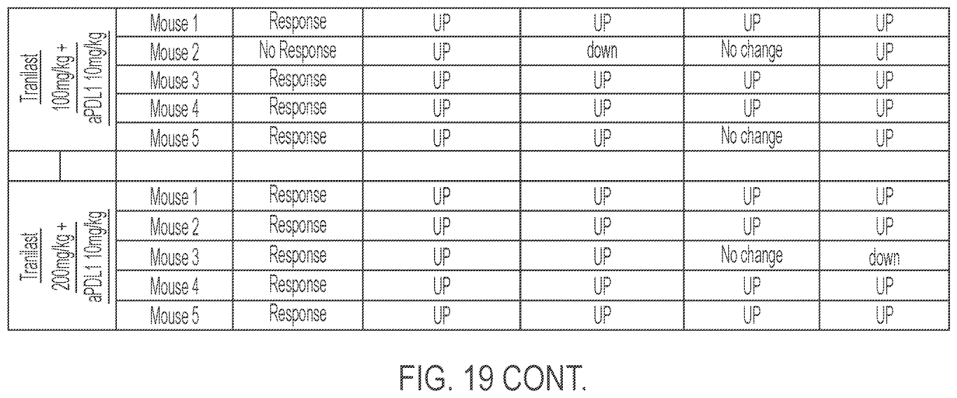

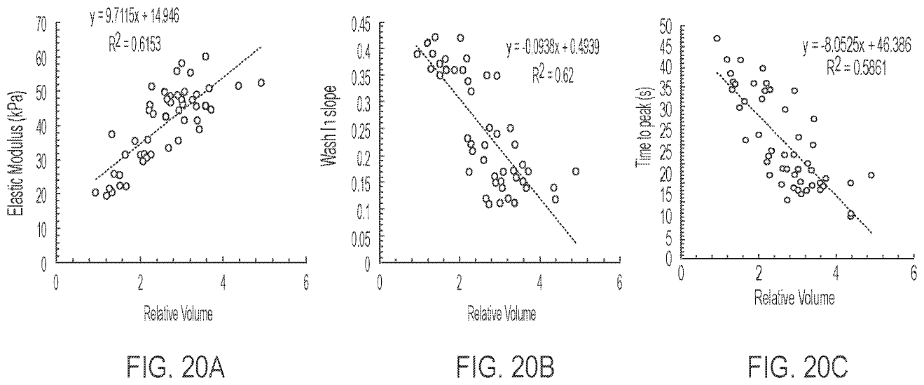

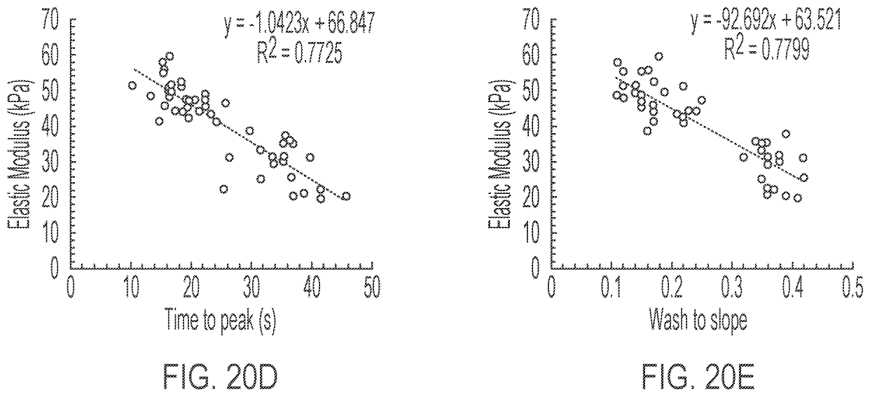

Additional correlations for tranilast-alone compared with tranilast with anti-PD-L1 therapy are shown in FIG. 16A-16E and FIG. 17 for mice bearing MCA205 tumors. Correlations for tranilast-alone compared with tranilast with anti-PD-L1 therapy are shown in FIG. 18A-18E and FIG. 19 for mice bearing E0771 tumors. In both tumor models, 100 mg/kg or 200 mg/kg tranilast pretreatment significantly potentiated tumors to immunotherapy. FIG. 20A-20E show the correlations for both models (mice bearing MCA205 or E0771 tumors). Together, these examples show that agents which reduce tumor stiffness and increase tumor perfusion can potentiate tumors to immunotherapy.

### Example 6: Additional Tumor Modulating Agent

This example shows the effect of another agent that decompresses blood vessels. Two mouse models of distinct sarcoma subtypes, fibrosarcoma (MCA205 cells) and osteosarcoma (K7M2 wt cells), were used in this study.

MCA205, a mouse fibrosarcoma cell line (SCC173, Millipore), was cultured in RPMI-1640 expansion medium containing 2 mM L-glutamine, 1 mM sodium pyruvate, 10% fetal bovine serum, 1× non-essential amino acids (TMS-001-C, Sigma), 1% antibiotics (A5955, Sigma), and 1× β-mercaptoethanol. K7M2 wt, a mouse osteosarcoma cell line (CRL2836™, ATCC®), was cultured in DMEM expansion medium supplemented with 10% FBS and 1% antibiotics. All cells were maintained at 37° C./5% CO₂.

A fibrosarcoma syngeneic tumor model was generated by subcutaneous implantation of 2.5×10⁵ MCA205 cells in 50 μL of serum-free medium into the flank of 6-week old C57BL/6 female mice. A osteosarcoma syngeneic tumor model was generated by implanting K7M2 wt tumor chunks into the fat pad of 6-week old BALB/c female mice.

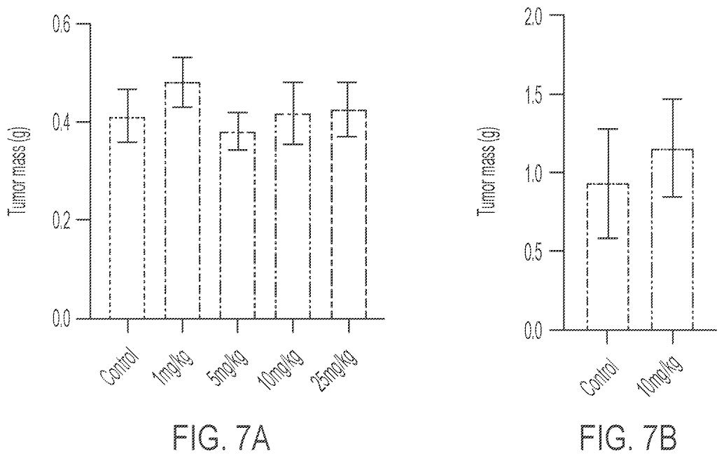

As shown in FIG. 7A (MCA205 tumors) and FIG. 7B (K7M2 wt tumors), ketotifen exhibited no antitumor effect by itself in either mouse sarcoma model.

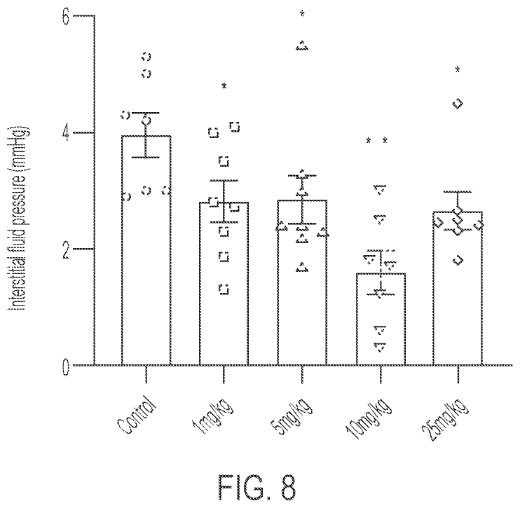

Interstitial fluid pressure (IFP) was measured in vivo using the “wick-in-needle” technique after mice were anesthetized with i.p. injection of Avertin and prior to tumor excision. Additional information regarding the wick-in-needle technique is described in Dong et al., Involvement of mast cell chymase in burn wound healing in hamsters 2013; 5:643-7 and Shankaran et al. IFNγ and lymphocytes prevent primary tumour development and shape tumour immunogenicity 2001; 410:1107-11, the contents of which are incorporated herein by reference in their entirety. All doses of ketotifen reduced the IFP, with the 10 mg/kg dose exhibiting the greatest effect (FIG. 8).

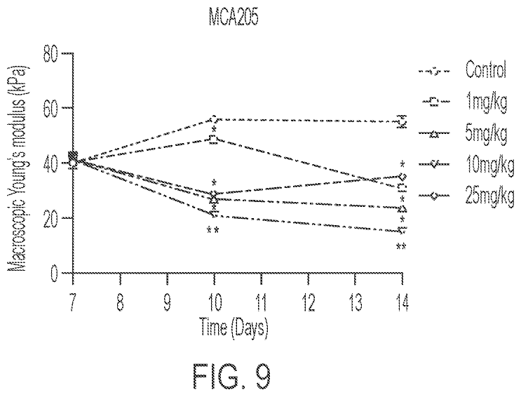

The mechanotherapeutic potential of ketotifen in reducing matrix stiffness was primarily assessed non-invasively and longitudinally using ultrasound elastography. Assessment of tumor elastic modulus was elastic via shear wave elastography using a Philips EPIQ Elite Ultrasound scanner with an eL 18-4 linear array. A dose of 10 mg/kg ketotifen reduced tissue stiffness the most in mice bearing MCA205 fibrosarcoma tumors, with Young's modulus values reaching 20 kPa, resembling the elasticity of healthy tissue (FIG. 9).

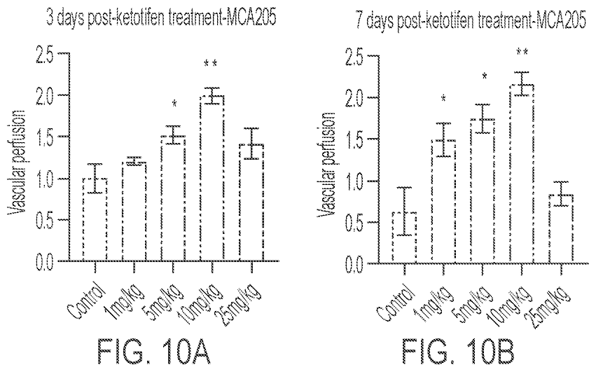

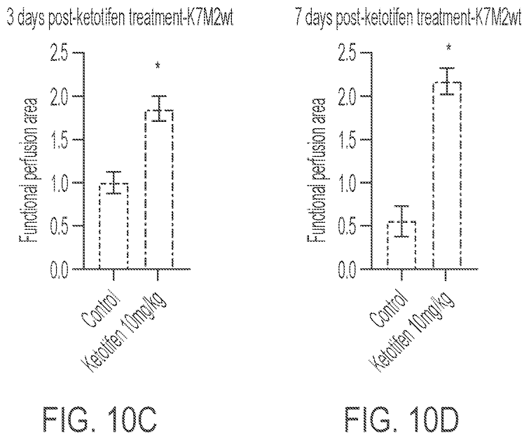

Vascular perfusion and functional perfusion were measured simultaneously during the course of ketotifen treatment, using contrast-enhanced ultrasound in mice bearing MCA205 and mice bearing K7M2 wt tumors. As shown in FIG. 10A-10D, ketotifen caused a significant increase in vascular and functional perfusion in both sarcoma subtypes.

Showing the above effects of ketotifen in the mice tumor models, the effect of ketotifen on the antitumor immune response of a chemotherapeutic and anti-PD-L1 checkpoint inhibitor was assessed.

Mice bearing sarcoma tumors were pre-treated with 10 mg/kg daily ketotifen followed by three or four doses of doxorubicin and/or an immune checkpoint inhibitor anti-PD-L1 antibody. Mouse monoclonal anti-PD-L1 (B7-H1, clone 10F.9G2, BioXCell) was used. Doxorubicin hydrochloride was prepared as a ready-made solution of 2 mg/ml. Anti-PD-L1 antibody was administered at a final dose of 10 mg/kg and doxorubicin at 5 mg/kg.

Mice bearing MCA205 tumors were pretreated with daily ketotifen 10 mg/kg or equal volume of diluent (control group) once the average tumor size reached 40 mm³, prior to the neoadjuvant treatment. Doxorubicin and anti-PD-L1 combination treatment initiated when tumors reached an average size of 150 mm³ (day 7) and was administered as i.p. injections every three days (day 7, 10, and 13) for three doses. Daily ketotifen continued until completion of doxorubicin-anti-PD-L1 combination treatment.

Primary tumors were resected and stored when they reached an average size of 700 mm³ on day 16 and mice were monitored for re-challenge experiments. Similarly, K7M2 wt tumors were pretreated with daily ketotifen 10 mg/kg or equal volume of diluent (control group) once the average tumor size reached 80 mm³ (day 18) and continued until completion of neoadjuvant treatment. Doxorubicin and anti-PD-L1 combination treatment started when tumors reached an average size of 150 mm³ (day 22) and repeated on day 25, 28 and 31. Study ended when tumors reached a mean volume of 550 mm 3 (day 33). Mice were sacrificed and tumors were collected for ex vivo analysis.

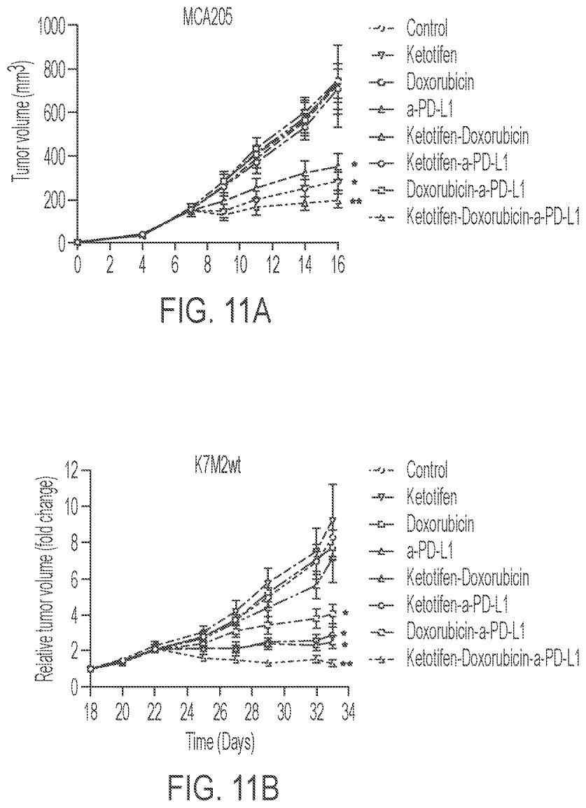

Neither anti-PD-L1 nor doxorubicin monotherapies significantly affected tumor growth of MCA205 or K7M2 wt tumors, while their combination with ketotifen induced a significant antitumor response (FIG. 11A for MCA205 tumors; FIG. 11B for K7M2 wt tumors).

What is claimed is:

1. A method for treating a solid tumor in a subject in need thereof comprising administering to the subject an effective amount of bosentan, or a pharmaceutically acceptable salt thereof, in combination with a checkpoint inhibitor.

2. A method for initiating, enhancing or prolonging the effects of a checkpoint inhibitor, or enabling a subject to respond to a checkpoint inhibitor in a subject in need thereof comprising administering to the subject an effective amount of bosentan, or a pharmaceutically acceptable salt thereof, in combination with a checkpoint inhibitor, wherein the subject has a solid tumor.

3. A method for potentiating the effects of a checkpoint inhibitor in a subject in need thereof comprising administering to the subject an effective amount of bosentan, or a pharmaceutically acceptable salt thereof, in combination with a checkpoint inhibitor, wherein the subject has a solid tumor.

4. A method of increasing blood flow of a solid tumor in a subject comprising administering to the subject an effective amount of bosentan, or a pharmaceutically acceptable salt thereof, in combination with a checkpoint inhibitor, wherein increasing blood flow of the solid tumor enhances the effect of the checkpoint inhibitor.

5. The method of claim 4, wherein blood flow is measured using ultrasound-based blood flow measurements or using histological techniques to measure hypoxia.

6. A method of improving the delivery or efficacy of a checkpoint inhibitor in a subject comprising administering an effective amount of bosentan, or a pharmaceutically acceptable salt thereof, in combination with the checkpoint inhibitor, wherein the subject has a solid tumor, thereby improving the delivery or efficacy of the therapy in the subject.

7. The method of any one of claims 1-6, wherein administering bosentan, or pharmaceutically acceptable salt thereof, increases the number of anti-tumor T cells that colocalize with the solid tumor.

8. The method of any one of claims 1-7, wherein administering bosentan, or pharmaceutically acceptable salt thereof, reduces the tissue stiffness of the solid tumor.

9. The method of claim 8, wherein the tissue stiffness of the solid tumor is measured using ultrasound elastography.

10. The method of any one of claims 1-9, wherein administering bosentan, or pharmaceutically acceptable salt thereof, decreases the levels of an extracellular matrix protein in the solid tumor.

11. The method of claim 10, wherein the extracellular matrix protein is collagen I or hyaluronan binding protein (HABP).

12. The method of any one of claims 1-11, wherein administering bosentan, or pharmaceutically acceptable salt thereof, reduces hypoxia in the solid tumor.

13. The method of any one of claims 1-12, wherein the checkpoint inhibitor inhibits a checkpoint protein selected from the group consisting of CTLA-4, PD-1, PD-L1, PD-L2, B7-H3, B7-H4, BMA, HVEM, TIM3, GALS, LAG3, VISTA, KIR, 2B4, CD160, CGEN-15049, CHK1, CHK2, A2aR, and B-7 family ligands or a combination thereof.

14. The method of claim 13, wherein the checkpoint inhibitor is a CTLA-4, PD-L1, PD-L2, or PD-1 inhibitor.

15. The method of claim 13, wherein the checkpoint inhibitor is an anti-PD-1 antibody, anti-PD-L1 antibody, or anti-CTLA4 antibody.

16. The method of claim 13, wherein the checkpoint inhibitor is selected from the group consisting of MEDI0680, AMP-224, nivolumab, pembrolizumab, pidilizumab, MEDI4736, atezolizumab, ipilimumab, tremelimumab, and BMS-936559.

17. The method of any one of claim 13-16, wherein the checkpoint inhibitor is a combination of an anti-PD-1 antibody and an anti-CTLA-4 antibody.

18. The method of any one of claims 1-17, wherein the bosentan, or pharmaceutically acceptable salt thereof, is administered to the subject once per day.

19. The method of any one of claims 1-17, wherein the bosentan, or pharmaceutically acceptable salt thereof, is administered to the subject twice per day.

20. The method of any one of claims 1-19, wherein the bosentan, or pharmaceutically acceptable salt thereof, is administered to the subject at a dose from about 0.01 mg/kg to about 5 mg/kg.

21. The method of any one of claims 1-19, wherein the bosentan, or pharmaceutically acceptable salt thereof, is administered to the subject at a dose from about 100 mg to about 1200 mg.

22. The method of any one of claims 1-19, wherein the bosentan, or pharmaceutically acceptable salt thereof, is administered to the subject at a dose from about 125 mg to about 500 mg.

23. The method of any one of claims 1-19, wherein the bosentan, or pharmaceutically acceptable salt thereof, is administered to the subject at a dose of about 125 mg.

24. The method of any one of claims 1-19, wherein the bosentan, or pharmaceutically acceptable salt thereof, is administered to the subject at a dose of about 500 mg.

25. The method of any one of claims 1-24, wherein the bosentan, or pharmaceutically acceptable salt thereof, is administered to the subject prior to the subject being administered the checkpoint inhibitor.

26. The method of claim 25, wherein the bosentan, or pharmaceutically acceptable salt thereof, is administered to the subject beginning at least 1 day prior to the subject being administered the checkpoint inhibitor.

27. The method of claim 25, wherein the bosentan, or pharmaceutically acceptable salt thereof, is administered to the subject beginning at least 2 days prior to the subject being administered the checkpoint inhibitor.

28. The method of claim 25, wherein the bosentan, or pharmaceutically acceptable salt thereof, is administered to the subject beginning at least 3 days prior to the subject being administered the checkpoint inhibitor.

29. The method of claim 25, wherein the bosentan, or pharmaceutically acceptable salt thereof, is administered to the subject beginning at least 5 days prior to the subject being administered the checkpoint inhibitor.

30. The method of any one of claims 1-29, wherein the administration of bosentan, or pharmaceutically acceptable salt thereof, to the subject is maintained for at least a portion of the time the subject is administered the checkpoint inhibitor.

31. The method of claim 30, wherein the administration of bosentan, or pharmaceutically acceptable salt thereof, to the subject is maintained for the entire period of time the subject is administered the checkpoint inhibitor.

32. The method of any one of claims 1-31, wherein one or more therapeutic effects in the subject is improved after administration of the bosentan, or pharmaceutically acceptable salt thereof, and the checkpoint inhibitor relative to a baseline.

33. The method of claim 32, wherein the one or more therapeutic effects is selected from the group consisting of: size of a tumor derived from the cancer, objective response rate, duration of response, time to response, progression free survival and overall survival.

34. The method of any one of claims 1-33, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the bosentan, or pharmaceutically acceptable salt thereof, and the checkpoint inhibitor.

35. The method of any one of claims 1-34, wherein the objective response rate is at least about 20%, at least about 25%, at least about 30%, at least about 35%, at least about 40%, at least about 45%, at least about 50%, at least about 60%, at least about 70%, or at least about 80%.

36. The method of any one of claims 1-35, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the bosentan, or pharmaceutically acceptable salt thereof, and the checkpoint inhibitor.

37. The method of any one of claims 1-36, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the bosentan, or pharmaceutically acceptable salt thereof, and the checkpoint inhibitor.

38. The method of any one of claims 1-37, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the bosentan, or pharmaceutically acceptable salt thereof, and the checkpoint inhibitor.

39. The method of any one of claims 1-38, wherein the solid tumor is selected from the group consisting of breast cancer, breast cancer lung metastases, sarcoma, pancreatic cancer, ovarian cancer, liver metastases, prostate cancer, brain cancer, melanoma, mesothelioma, renal cell carcinoma, colorectal cancer, hepatocellular carcinoma, lung cancer, squamous cell carcinoma of the head and neck, urothelial carcinoma, esophageal squamous cell carcinoma, gastric cancer, esophageal cancer, cervical cancer, Merkel cell carcinoma, endometrial carcinoma, and cutaneous squamous cell carcinoma.

40. The method of claim 39, wherein the solid tumor is breast cancer.

41. The method of claim 40, wherein the breast cancer is triple negative breast cancer.

42. The method of any one of claims 1-41, wherein the subject is a human.

43. A kit comprising:
(a) an effective amount of bosentan, or a pharmaceutically acceptable salt thereof;
(b) an effective amount of a checkpoint inhibitor; and
(c) instructions for using the bosentan, or a pharmaceutically acceptable salt thereof and the checkpoint inhibitor according to the method of any one of claims 1-42.

44. A method of determining an effective amount of an agent that decompresses blood vessels in a subject with a solid tumor comprising:
(a) measuring the blood flow and/or stiffness of the solid tumor;
(b) administering to the subject an effective amount of an agent that decompresses blood vessels; and
(c) measuring the blood flow and/or stiffness of the solid tumor after administering the agent that decompresses blood vessels, wherein an increase in blood flow and/or a decrease in stiffness following administration of the agent that decompresses blood vessels to the subject indicates that the amount administered was an effective amount.

45. A method for treating a solid tumor in a subject in need thereof comprising:
(a) measuring the blood flow and/or stiffness of the solid tumor;
(b) administering to the subject an effective amount of an agent that decompresses blood vessels;
(c) measuring the blood flow and/or stiffness of the solid tumor after administering the agent that decompresses blood vessels; and
(d) administering a chemotherapeutic agent if the blood flow of the solid tumor is increased and/or the stiffness of the solid tumor is decreased after administering the agent that decompresses blood vessels.

46. A method for treating a solid tumor in a subject in need thereof comprising:
(a) measuring the blood flow and/or stiffness of the solid tumor;
(b) administering to the subject an effective amount of an agent that decompresses blood vessels;
(c) measuring the blood flow and/or stiffness of the solid tumor after administering the agent that decompresses blood vessels;
(d) determining that the subject is responsive to a chemotherapeutic agent based on an increase in the blood flow of the solid tumor or a decrease in the stiffness of the solid tumor after administering the agent that decompresses blood vessels; and
(e) administering the chemotherapeutic agent to the subject who has been determined to be responsive to the chemotherapeutic agent based on the increase in the blood flow of the solid tumor or the decrease in the stiffness of the solid tumor after administering the agent that decompresses blood vessels.

47. A method for predicting response to treatment with a chemotherapeutic agent comprising:
(a) measuring the blood flow and/or stiffness of the solid tumor;
(b) administering to the subject an effective amount of an agent that decompresses blood vessels;
(c) measuring the blood flow and/or stiffness of the solid tumor after administering the agent that decompresses blood vessels,
wherein an increase in the blood flow of the solid tumor or a decrease in the stiffness of the solid tumor after administering the agent that decompresses blood vessels indicates that the subject is likely to respond to treatment with the chemotherapeutic agent.

48. The method of any one of claims 45-47, wherein the effective amount of an agent that decompresses blood vessels is determined by measuring the change in blood flow and/or stiffness of the solid tumor following administration of the agent that decompresses blood vessels to the subject, wherein an increase in blood flow and/or a decrease in stiffness following administration of the agent that decompresses blood vessels to the subject indicates that the amount administered was an effective amount.

49. The method of any one of claims 44-48, wherein the method comprises measuring the blood flow of the solid tumor and the blood flow of the solid tumor is increased after administering the agent that decompresses blood vessels.

50. The method of any one of claims 44-48, wherein the method comprises measuring the stiffness of the solid tumor and the stiffness of the solid tumor is decreased after administering the agent that decompresses blood vessels.

51. The method of any one of claims 44-50, wherein the agent that decompresses blood vessels is administered for at least 1 day, at least 2 days, at least 3 days, at least 4 days, or at least 5 days prior to the administration of the chemotherapeutic agent.

52. The method of any one of claims 44-51, wherein the agent that decompresses blood vessels is administered at a dose that increases the blood flow of the solid tumor and/or decreases the stiffness of the solid tumor.

53. The method of any one of claims 44-52, wherein the agent that decompresses blood vessels is bosentan, or a pharmaceutically acceptable salt thereof.

54. The method of any one of claims 44-53, wherein blood flow and/or stiffness of the solid tumor is measured using ultrasound.

55. The method of claim any one of claims 44-53, wherein blood flow of the solid tumor is measured using histological techniques to measure hypoxia.

56. The method of any one of claims 45-55, wherein the chemotherapeutic agent is a checkpoint inhibitor.

57. The method of claim 56, wherein the checkpoint inhibitor inhibits a checkpoint protein selected from the group consisting of CTLA-4, PD-1, PD-L1, PD-L2, B7-H3, B7-H4, BMA, HVEM, TIM3, GALS, LAG3, VISTA, KIR, 2B4, CD160, CGEN-15049, CHK1, CHK2, A2aR, and B-7 family ligands or a combination thereof.

58. The method of claim 57, wherein the checkpoint inhibitor is a CTLA-4, PD-L1, PD-L2, or PD-1 inhibitor.

59. The method of claim 57, wherein the checkpoint inhibitor is an anti-PD-1 antibody, anti-PD-L1 antibody, or anti-CTLA4 antibody.

60. The method of claim 57, wherein the checkpoint inhibitor is selected from the group consisting of MEDI0680, AMP-224, nivolumab, pembrolizumab, pidilizumab, MEDI4736, atezolizumab, ipilimumab, tremelimumab, and BMS-936559.

61. The method of any one of claim 57-60, wherein the checkpoint inhibitor is a combination of an anti-PD-1 antibody and an anti-CTLA-4 antibody.

62. The method of any one of claims 44-61, wherein the solid tumor is selected from the group consisting of breast cancer, breast cancer lung metastases, sarcoma, pancreatic cancer, ovarian cancer, liver metastases, prostate cancer, brain cancer, melanoma, renal cell carcinoma, colorectal cancer, hepatocellular carcinoma, lung cancer, squamous cell carcinoma of the head and neck, urothelial carcinoma, esophageal squamous cell carcinoma, gastric cancer, esophageal cancer, cervical cancer, Merkel cell carcinoma, endometrial carcinoma, mesothelioma, and cutaneous squamous cell carcinoma.

63. The method of claim 62, wherein the solid tumor is breast cancer.

64. The method of claim 63, wherein the breast cancer is triple negative breast cancer.

65. The method of any one of claims 44-64, wherein the subject is a human.
